(19) United States
(12) Patent Application Publication
(10) Pub. No.: US 2015/0315621 A1
(43) Pub. Date: Nov. 5, 2015

(54) ONE-POT FERMENTATION PROCESS FOR SIMVASTATIN PRODUCTION

(71) Applicant: THE REGENTS OF THE UNIVERSITY OF CALIFORNIA, Oakland, CA (US)

(72) Inventors: Yi Tang, San Gabriel, CA (US); Wei Xu, Los Angeles, CA (US); Yit Heng Chooi, Franklin (AU)

(73) Assignee: THE REGENTS OF THE UNIVERSITY OF CALIFORNIA, Oakland, CA (US)

(21) Appl. No.: 14/700,758

(22) Filed: Apr. 30, 2015

Related U.S. Application Data
(60) Provisional application No. 61/986,304, filed on Apr. 30, 2014.

(51) Int. Cl. C12P 17/06; C12N 9/02; C12N 9/16; C12N 9/10

(52) U.S. Cl. CPC C12P 17/06; C12N 9/1029; C12N 9/001; C12N 9/16; C12Y 203/01161; C12Y 103/01009

(57) ABSTRACT

Simvastatin is a drug used in treatment of hypercholesterolemia because of its inhibitory activities towards cholesterol biosynthesis. The technology described herein is a novel method of biosynthesizing Simvastatin directly from an engineered microbial host (such as Saccharomyces cerevisiae) using an engineered biosynthetic pathway. The identification and engineering of Aspergillus terreus biosynthetic pathways opens up new metabolic engineering opportunities for statin production from organisms such as yeast. Using this technology, Simvastatin can be obtained by feeding a simple synthetic building block to fermented culture. Such methods provide a cost-effective way of obtaining Simvastatin including, for example, one-pot fermentation processes.

### CROSS-REFERENCE TO RELATED APPLICATIONS

This application claims the benefit under 35 U.S.C. Section 119(e) of co-pending U.S. Provisional Patent Application Ser. No. 61/986,304, titled “ONE-POT FERMENTATION PROCESS FOR SIMVASTATIN PRODUCTION FROM SACCHAROMYCES CEREVISIAE”, filed Apr. 30, 2014, the contents of which are incorporated herein by reference. This application is related to U.S. Pat. Nos. 8,211,664 and 8,981,056, the contents of which are incorporated herein by reference.

### STATEMENT AS TO RIGHTS TO INVENTIONS MADE UNDER FEDERALLY SPONSORED RESEARCH AND DEVELOPMENT

This invention was made with Government support under GM089998, awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Apr. 21, 2015, is named 30435.270-US-U1_SL.txt and is 107,906 bytes in size.

### TECHNICAL FIELD

The invention relates to methods and materials for biosynthesizing compounds such as simvastatin including procedures using microbial hosts.

### BACKGROUND OF THE INVENTION

Simvastatin is a semisynthetic derivative of the natural product lovastatin, which can be isolated from the fermentation broth of Aspergillus terreus. Both lovastatin and simvastatin are cholesterol lowering drugs that substantially lower the risk of heart disease among adults. Lovastatin and simvastatin are marketed by Merck Co. as Mevacor and Zocor, respectively. Simvastatin is a more potent derivative of lovastatin and is one of the best selling drug in the United States.

The gene cluster for lovastatin biosynthesis in A. terreus (see, e.g., J. Kennedy, K. et. al., Science, 1999, 284, 1368-1372; and C. R. Hutchinson, J. et. al., Antonie Van Leeuwenhoek 2000, 78, 287-295) has been described previously (see, e.g., U.S. Pat. No. 6,391,583, the contents of which are herein incorporated by reference). Encoded in the gene cluster is a 46 kD protein LovD, that was initially identified as an esterase homolog. Monacolin J, the immediate biosynthetic precursor of lovastatin, is assembled by the upstream megasynthase LovB (see, e.g., L. Hendrickson, C. R. et. al., Chem. Biol. 1999, 6, 429-439), (also known as lovastatin nonaketide synthase, LNKS), enoylreductase LovC and CYP450 oxygenases. The five carbon unit side chain is synthesized by LovF (lovastatin diketide synthase, LDKS) through condensation between an acetyl-CoA and a malonyl-CoA. The condensed diketide undergoes methylation and reductive tailoring by the individual LovF domains to yield an α-S-methylbutyryl thioester covalently attached to the phosphopantetheine arm on the acyl carrier protein (ACP) domain of LovF (see, e.g., J. Kennedy, K. et. al., Science, 1999, 284, 1368-1372 and C. R. Hutchinson, J. et. al., Antonie Van Leeuwenhoek 2000, 78, 287-295), and Lovastatin is subsequently produced from monacolin J. Inactivation of either LovD or LovF in A. terreus leads to accumulation of the precursor monacolin J (see, e.g., J. Kennedy, K. et. al., Science, 1999, 284, 1368-1372 and C. R. Hutchinson, J. et. al., Antonie Van Leeuwenhoek 2000, 78, 287-295).

Various multistep synthesis of simvastatin have been described previously (see, e.g., PCT WO 2005/066150 and U.S. Application Nos. 20050080275 and 20040068123, the contents of which are herein incorporated by reference). Currently, industrial production of simvastatin is based on chemical modification of purified lovastatin, a natural product from a fungus, Aspergillus terreus. This is achieved through the replacement of the 2-methylbutyryl side chain with a 2,2-dimethylbutyryl group after multiple reactions. Such a process first requires the isolation of lovastatin (or its important biosynthetic precursor monacolin J) from the fermentation broth of Aspergillus terreus. After purification of lovastatin, the semi-synthesis process includes: 1) hydrolysis of lovastatin, removal of the dikeitede 2-methylbutyrate side arm from the nonaketide core to yield monacolin J acid; 2) lactonization of the monacolin J acid; 3) protection of the C13 hydroxyl of monacolin J lactone; 4) esterification of the exposed C8 hydroxyl with 2,2-dimethylbutyryl chloride to yield a C13 protected version of simvastatin; and 5) deprotection on C13 hydroxyl to yield simvastatin.

Variations of the above schemes are common, however, most procedures will invariably involve isolation of lovastatin first, hydrolysis of the methylbutyrate side chain, protection of the free alcohol, reaction with an acyl substrate, and deprotection. Although the chemical transformations involved are relatively simple, they are inefficient and involve multiple steps and therefore contribute to the current high cost of manufacturing simvastatin ($3 per pill). As disclosed herein, Steps 2) to 5) above can be replaced by a one-step fermentation process. The acyl transferase in the lovastatin biosynthetic pathway (LovD) is also able to enzymatically convert monacolin J to simvastatin when a 2,2-dimethylbutyryl donor (like dimethylbutyryl-S-methylmercaptopropionate) is provided.

### SUMMARY OF THE INVENTION

The present invention provides simplified procedures for producing simvastatin and its precursors. The disclosure below includes, for example, methods and materials for biosynthesizing monacolin J and/or simvastatin directly from an engineered microbial host in a single fermentation vessel.

The invention disclosed herein has a number of embodiments. One embodiment of the invention is a method of making simvastatin in a microbial host organism (e.g. Saccharomyces cerevisiae) that has been transduced with a constellation of heterologous genes in order to, for example, produce monacolin J. In these methods, the microbial host organism comprises heterologous genes that express LovA (SEQ ID NO: 1), LovB (SEQ ID NO: 2), LovC (SEQ ID NO: 3), LovD (SEQ ID NO: 4), LovG (SEQ ID NO: 5), and/or cytochrome P450 oxidoreductase (SEQ ID NO: 6) and produces monacolin J. In these methods, the microbial host organism is combined with a 2,2-dimethylbutyryl donor compound so that LovD transfers the dimethylbutyryl group from the 2,2-dimethylbutyryl donor compound to regioselectively acylate the C8 hydroxyl group of monacolin J, thereby making simvastatin. In typical embodiments of the invention, the simvastatin is formed in a one-pot fermentation process.

Microbial host organisms useful in embodiments of the invention include Saccharomyces cerevisiae, Escherichia coli, Monascus ruber, Monascus purpureus, Monascus pilosus, Monascus vitreus, Monascus pubigerus, Candida cariosilognicola, Aspergillus oryzea, Doratomyces stemonitis, Paecilomyces virioti, Penicillum citrinum, Penicillin chrysogenum, Scopulariopsis brevicaulis or Trichoderma viride. In illustrative embodiments of the invention, the microbial host organism is grown under at least one of the following conditions: (a) at a temperature between 30-40° C.; (b) for a time period between at least 4 to at least 48 hours; (c) at a pH between 7-8; or (d) in a fermentation media comprising YPD, LYPD, YNB, HC or YC media. In addition, the methods can further comprising purifying the simvastatin made by the method by at least one purification step comprising: (a) lysis of cells of an isolated organism present in the combination; (b) centrifugation; (c) precipitation of a free acid form of simvastatin; (d) conversion of a free acid form of simvastatin to a simvastatin salt; (e) filtration; or (f) high performance liquid chromatography (HPLC).

In some embodiments of the invention, one or more genes expressing LovA (SEQ ID NO: 1), LovB (SEQ ID NO: 2), LovC (SEQ ID NO: 3), LovD (SEQ ID NO: 4), LovG (SEQ ID NO: 5), and cytochrome P450 oxidoreductase (SEQ ID NO: 6) is codon optimized. Optionally, these genes are expressed episomally in at least two separate vectors in the microbial host organism. In some embodiment of the invention, the 2,2-dimethylbutyryl donor compound is selected to possess at least one of the following properties: (a) is a butyrlyl-thioester, a N-acetylcysteamine thioester or a methyl-thioglycolate thioester; (b) comprises medium chain length (C3-C6) acyl group moieties; (c) is able to cross the cellular membranes of Escherichia coli or Aspergillus terreus cells growing within a fermentation media; or (d) is selected from the group consisting of α-dimethylbutyryl-S-methyl-mercaptopropionate (DMB-S-MMP), dimethylbutyryl-S-ethyl mercaptopropionate (DMB-S-EMP) and dimethylbutyryl-S-methyl thioglycolate (DMB-S-MTG) and dimethylbutyryl-S-methyl mercaptobutyrate (DMB-S-MMB).

Embodiments of the invention include methods and materials for making monacolin J, a simvastatin precursor molecule. One such embodiment is a method of making monacolin J comprising the steps of combining a first microbial host organism (e.g. Saccharomyces cerevisiae) with a growth medium, wherein the microbial host organism comprises one or more heterologous genes expressing LovA (SEQ ID NO: 1), LovB (SEQ ID NO: 2), LovC (SEQ ID NO: 3), LovG (SEQ ID NO: 5), and cytochrome P450 oxidoreductase (SEQ ID NO: 6) so that monacolin J is made. Typically in such embodiments, monacolin j is made in concentrations of at least 10 mg/L. Embodiments of these methods can further comprise converting the Monacolin J to simvastatin by coculturing the first microbial host organism with a second microbial host organism comprising a gene expressing LovD (SEQ ID NO: 4); and a 2,2-dimethylbutyryl donor compound.

Yet another embodiment of the invention is a composition of matter comprising a microbial host organism wherein the microbial host organism comprises one or more heterologous genes expressing LovA (SEQ ID NO: 1), LovB (SEQ ID NO: 2), LovC (SEQ ID NO: 3), LovG (SEQ ID NO: 5), and cytochrome P450 oxidoreductase (SEQ ID NO: 6); such that the microbial host organism can produce monacolin J during fermentation. Optionally the composition further comprises monacolin J. The compositions can also further comprise a culture media in which the microbial host organism produces monacolin J during fermentation. Certain compositions useful in making simvastatin further include an organism comprising LovD (SEQ ID NO: 4), monacolin J, simvastatin and/or a 2,2-dimethylbutyryl donor compound.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. Lovastatin biosynthetic gene cluster and pathway.

FIG. 2. Metabolic profiles of fermentation extracts from cultures of different A. terreus strains. A) HPLC chromatograms at 238 nm and the extracted ion chromatograms of observed masses m/z [M+Na]⁺ 445 corresponding to 1 in wild type A. terreus. B) HPLC chromatograms at 238 nm and the extracted ion chromatograms of observed masses m/z [M+Na]⁺ 445 corresponding to 1 in ΔlovG A. terreus mutant. The traces are drawn to the same scale.

FIG. 3. LovG releases 2 from LovB. A) HPLC chromatogram at 200 nm of fermentation extracts from 3-day cultures of BJ5464-NpgA expressing i) LovB, LovC and LovG; ii) LovB and LovC; and B) Extracted ion chromatograms of m/z [M-H]⁻ 323 corresponding to 2 in in vitro experiments containing i) LovB, LovC and LovG; ii) LovB and LovC, treated with 1M KOH; iii) LovB and LovC with no 1M KOH treatment. All enzymes are added to a final concentration of 10 μM. The reactions were performed at 25° C. for 12 hours.

FIG. 4. Production of 3 from S. cerevisiae BJ5464-NpgA co-expressing LovB, LovC, LovG, LovA and A. terreus cytochrome P450 oxidoreductase (CPR). i) Extracted ion chromatograms showing detection of 2, 3 and 4 in culture 48 hours after induction of LovA and CPR expression. m/z [M-H]⁻ for 2: 323, 3: 337 and 4: 321. ii-iv) HPLC Chromatograms at 238 nm of fermentation extracts. ii) 48 hours after induction of LovA and CPR expression; iii) 24 hours after induction of LovA and CPR expression iv) Before induction of LovA and CPR expression.

FIG. 5. LovG releases aberrant products from LovB. HPLC Chromatograms are extracted at 330 nm. A) in vitro assay with LovB; B) in vitro assay with LovB+LovG.

FIG. 6. Simvastatin Production from Yeast.

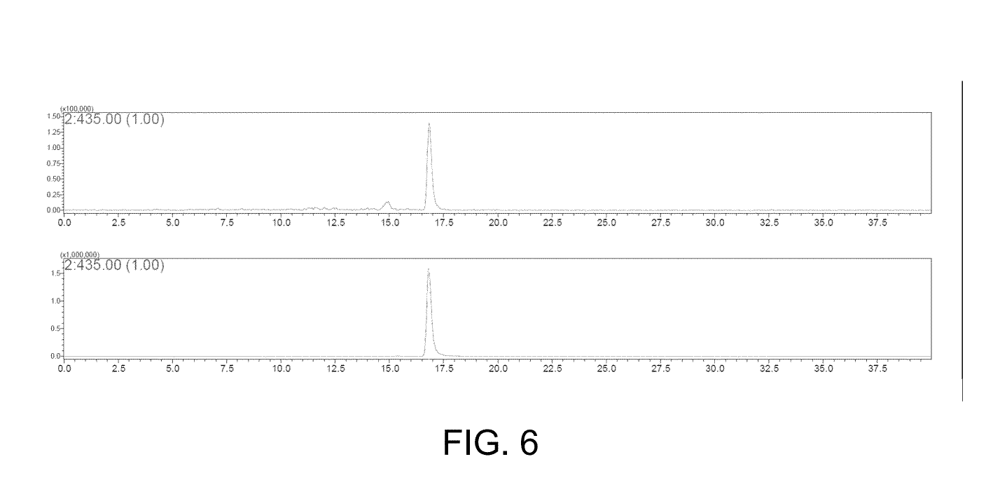

FIG. 7. A previously cryptic esterase-like enzyme (LovG) is found to be responsible for the product release from the lovastatin nonaketide synthase (LNKS or LovB). The same enzyme also helps improving turnover of LovB through hydrolysis of incorrectly tailored intermediates.

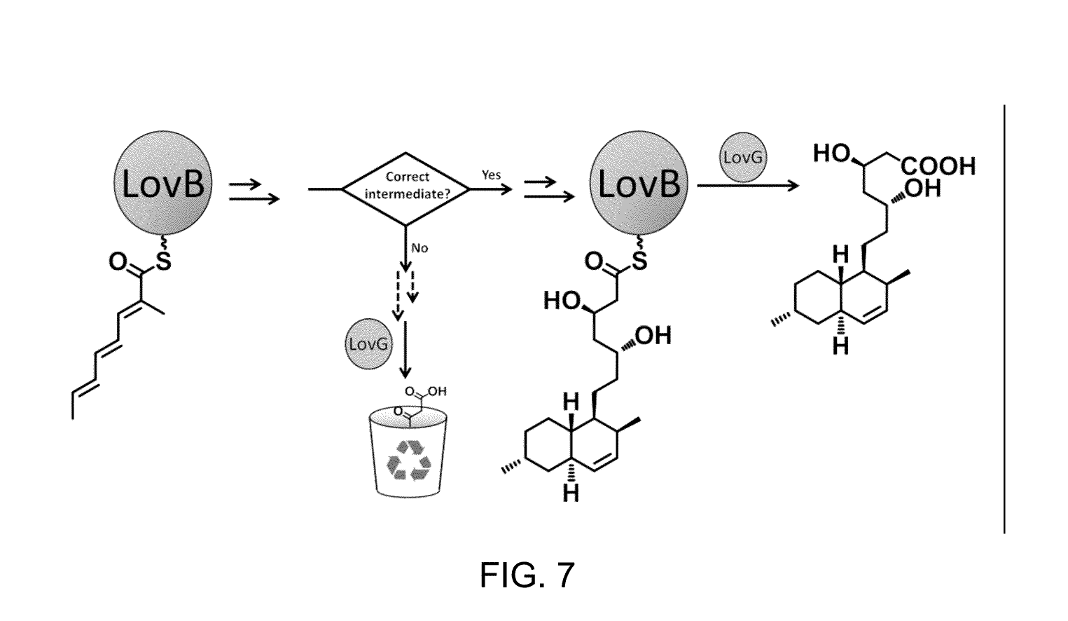

FIG. 8. Coexpression of LovB and LovC alone in BJ5464-NpgA cannot produce 2. A) HPLC trace at 200 nm. B) Extracted ion chromatogram for 2, m/z [M-H]⁻ 323.

FIG. 9. Time course of the DML acid (2) production in S. cerevisiae strain BJ5464-NpgA that cotransformed with lovB, lovC and heterologous TE genes.

FIG. 10. NCBI Conserved Domain Search result for LovG, showing that LovG belongs to serine hydrolase family. FIG. 10 discloses SEQ ID NOS 16 and 17, respectively, in order of appearance.

FIG. 11. Deletion of lovG from the by cluster. Knockout strategy employed for ΔlovG. Correct integration is verified by diagnostic PCR using the two sets of primers shown. (Primers: p1—LovG_KO_screen-f; p2—rev in BLE; p3—for in BLE; p4—LovG_KO-Screen-r)

FIG. 12. SDS PAGE gel images of the heterogeneously expressed LovG from E. coli, before and after size-exclusion chromatography.

FIG. 13. In vitro turnover assay for production of 2 by LovB, LovC and LovG. Paralelle reactions were taken place in 100 μL solution buffered at pH 7.4. Equimolar amounts (10 μM) LovG, LovB and LovC were incubated with necessary cofactors: 2 mM NADPH and 1 mM SAM. 20 μM MatB was used to replenish malonyl-CoA from 10 mM CoA and 100 mM malonate in presence of 20 mM ATP and 7 mM MgCl2. The produced 2 is quantified on LC-MS by comparing the MS peak area to the standard. The initial turnover rate was calculated using the first four data point that are in the linear region of the curve.

FIG. 14. Time course of the DML acid (2) production in S. cerevisiae strain BJ5464-NpgA that cotransformed with lovB, lovC and lovG genes. The sample culture was extracted twice with equal volume of 99% ethyl acetate (EA)/1% acetic acid (AcOH). The resultant organic extracts were evaporated to dryness, redissolved in 0.05M NaOH in methanol, and then analyzed on LC-MS. The produced 2 is quantified on LC-MS by comparing the MS peak area to the standard.

FIG. 15. LC-MS and UV data for compound 6 from LovB/LovG in vitro assay.

FIG. 16. LC-MS and UV data for compound 7 from LovB/LovG in vitro assay.

FIG. 17. LC-MS and UV data for compound 8 from LovB/LovG in vitro assay.

FIG. 18. LC-MS and UV data for compound 9 from LovB/LovG in vitro assay.

FIG. 19. LC-MS and UV data for compound 10 from LovB/LovG in vitro assay.

FIG. 20. Proofreading function of LovG in absence of LovC.

FIG. 21. Table of primers used in the Example 2. Table discloses SEQ ID NOS 18-33, respectively, in order of appearance.

FIG. 22. Table of the expression plasmids used in the Example 2. FIG. 22 discloses “hexahistidine” as SEQ ID NO: 15.

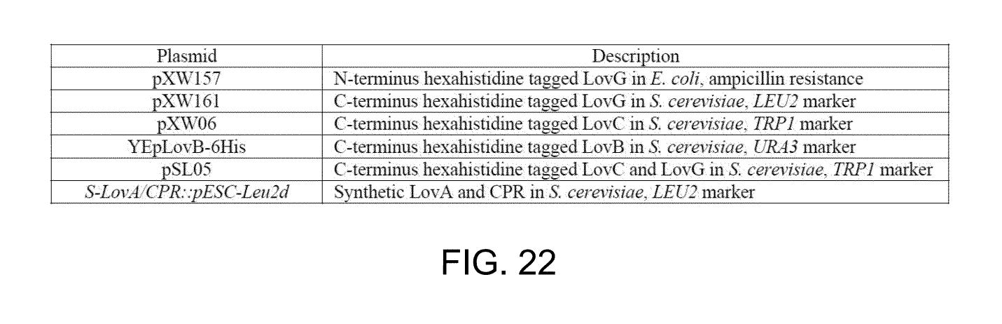

### DETAILED DESCRIPTION OF THE INVENTION

The techniques and procedures described or referenced herein are generally well understood and commonly employed using conventional methodology by those skilled in the art, such as, for example, the widely utilized molecular cloning methodologies described in Ausubel et al., Current Protocols in Molecular Biology, Wiley Interscience Publishers, (1995). As appropriate, procedures involving the use of commercially available kits and reagents are generally carried out in accordance with manufacturer defined protocols and/or parameters unless otherwise noted.

This application references a number of different publications as indicated throughout the specification by reference numbers enclosed in brackets, e.g., [x]. A list of these different publications ordered according to these reference numbers can be found below in the section entitled “References.” All publications mentioned herein (e.g. as U.S. Pat. No. 8,211,664, International Publication No. WO 2011/044496) are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further the actual publication dates may be different from those shown and require independent verification.

In the description of the preferred embodiment, reference is made to the accompanying drawings which form a part hereof, and in which is shown by way of illustration a specific embodiment in which the invention may be practiced. It is to be understood that other embodiments may be utilized and structural changes may be made without departing from the scope of the present invention.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The definitions of certain terms are provided as follows:

“Lovastatin” is a fungal polyketide produced by Aspergillus terreus (see, e.g., A. W. Alberts, J. et. al., Proc. Natl. Acad. Sci. U.S.A., 1980, 77, 3957-3961 and A. Endo, J. Antibiot. 1980, 33, 334-336; and J. K. Chan, et. al., J. Am. Chem. Soc. 1983, 105, 3334-3336; Y. Yoshizawa, et. al., J. Am. Chem. Soc. 1994, 116, 2693-2694). It is a pharmaceutically important compound because of its potent inhibitory activities towards hydroxymethylglutaryl coenzyme A reductase (HMGR), the rate-limiting step of cholesterol biosynthesis, and therefore it is widely used in the treatment of hyperlipidemia, hypercholesterolemia, and the like. Lovastatin is also referred to as Mevacor. “Simvastatin” is an analog of lovastatin. It is favored over lovastatin because of the absence of adverse side effects and its high absorbability in the stomach. Also, it has been reported that simvastatin prevents and reduces the risk of Alzheimer's disease (AD) by retarding the production of Ab42, β-amyloid protein associated with AD. In describing compounds such as simvastatin, pravastatin, monacolin J and variants etc., those of skill in the art understand that this language is intended to encompass these compounds as well as the salts of these compounds (e.g. pharmaceutically acceptable salts known in the art). For example, as is known in the art, simvastatin can occur both a free acid form as well as a simvastatin sodium, potassium or ammonium salts, and other salts derived from alkaline earth elements or other metallic salts.

As is known in the art, genes related to biosynthesis of secondary metabolites of filamentous fungi can form a cluster on the fungal genome and are referred to as “gene clusters.” For example, “Lovastatin-producing gene cluster” can refer to a set of genes that produce lovastatin, the set of genes comprising, LovA, a P450I; LovC, a dehydrogenase; LovD, an esterase and acyltransferase; and LovF, a ScPKS or LDKS. It has been determined previously that each of these four genes (LovA, LovC, LovD, and LovF) is required for lovastatin synthesis (see, e.g., U.S. Pat. No. 6,943,017, the contents of which are herein incorporated by reference). LovF (LDKS gene) is characterized as a polyketide synthase gene. LovD is a putative esterase/carboxypeptidase-like gene. Disruption of the LovF gene has been done previously (see, e.g., U.S. Pat. No. 6,943,017, the contents of which are herein incorporated by reference). LovD interacts with LovF to produce lovastatin; however, the LovD-LovF interaction is not required for the production of simvastatin. Moreover, another gene in the lovastatin-producing gene cluster is LovE, which is a Zn finger that can regulate the transcription of the other genes. The lovastatin-producing gene cluster also comprises LovB (NPKS gene).

“LDKS” or “LDKS gene” refers to the protein encoded by the LovF gene, a member of the lovastatin-producing gene cluster. LDKS stands for lovastatin diketide synthase. LovF is the gene that produces LDKS. LovF is also the gene that produces LovF protein. “LDKS gene” also refers to the gene that produces LDKS. In the synthesis of lovastatin, LDKS synthesizes the five carbon unit side chain of monacolin J through condensation between an acetyl-CoA and a malonyl-CoA. The condensed diketide undergoes methylation and reductive tailoring by the individual LovF domains to yield an α-S-methylbutyryl thioester covalently attached to the phosphopantetheine arm on the acyl carrier protein (ACP) domain of LovF.

“LovD acyltransferase” as used herein refers to those polypeptides such as the A. terreus LovD polypeptide (e.g. SEQ ID NO: 1) that can use a acyl thioester to regiospecifically acylate the C8 hydroxyl group of monacolin J so as to produce simvastatin. As also disclosed herein, this LovD enzyme can further utilize a acyl thioester to regiospecifically acylate the C8 hydroxyl group of hydrolyzed pravastatin tetra-ol so as to produce huvastatin.

LovD acyltransferases include homologous enzymes to A. terreus LovD polypeptide (e.g. SEQ ID NO: 1) that can be found in for example, but not limited to, fungal polyketide gene clusters. For example, the art provides evidence that Mlc in the compactin biosynthetic pathway catalyzes the identical transacylation reaction (see, e.g., Y. Abe, T. et. al., Mol Genet Genomics. 2002, 267, 636-646), whereas an acyltransferase in the squalestatin pathway can catalyze a similar reaction between an ACP-bound tetraketide thioester and an aglycon (see, e.g., R. J. Cox, F. et. al., Chem Commun (Camb) 2004, 20, 2260-2261). The amino acid sequence of A. terreus LovD polypeptide (e.g. SEQ ID NO: 1) resembles type C β-lactamase enzymes, which catalyze the hydrolytic inactivation of the β-lactam class of antibiotics (see., e.g., E. Lobkovsky, E. M. et. al., Biochemistry, 1994, 33, 6762-6772 and A. Dubus, D. et. al., Biochem. J. 1993, 292, 537-543). Alignment of A. terreus LovD polypeptide (e.g. SEQ ID NO: 1) with the enterobacter cloacae P99 lactamse (see, e.g., S. D. Goldberg, et. al., Protein Sci. 2003, 12, 1633-1645) shows moderate sequence homology, including potentially conserved active site residues, such as the catalytic Ser76, Lys79, Tyr188, and Lys315 (see. e.g. S. D. Goldberg, et. al., Protein Sci. 2003, 12, 1633-1645).

LovD acyltransferases can also refer to both genetically engineered and naturally occurring enzymes that are related to A. terreus LovD polypeptide (e.g. SEQ ID NO: 1) in sequence but containing slight amino acid differences (e.g. 1-10 amino acid substitution mutations). Simvastatin, for example, can be produced from naturally occurring enzymes that are similar to A. terreus LovD polypeptide (e.g. SEQ ID NO: 1) in sequence (e.g. the M1CH from the compactin cluster). “LovD acyltransferases” can also refer to mutants of A. terreus LovD polypeptide (SEQ ID NO: 1). It is known in the art that mutants can be created by standard molecular biology techniques to produce, for example, mutants of SEQ ID NO: 1 that improve catalytic efficiencies or the like. For example, we are currently using rational and directed evolution approaches to improve the catalytic turnover rates of A. terreus LovD. Typically such mutants will have a 50%-99% sequence similarity to SEQ ID NO: 1. In this context, the term “LovD homologous enzyme” includes a LovD polypeptide having at least 80%, 85%, 90%, 95%, 97%, 98% or 99% sequence identity with the amino acid sequence set out in SEQ ID NO: 1, wherein the polypeptide has the ability to utilize a acyl thioester to regiospecifically acylate the C8 hydroxyl group of monacolin J so as to produce simvastatin and/or utilize a acyl thioester to regiospecifically acylate the C8 hydroxyl group of hydrolyzed pravastatin tetra-ol so as to produce huvastatin. Such mutants are readily made and then identified in assays which observe the production of a desired compound such as simvastatin (typically using A. terreus LovD polypeptide (e.g. SEQ ID NO: 1) as a control). These mutants can be used by the methods of this invention to make simvastatin or huvastatin, for example.

“Heterologous” as it relates to nucleic acid sequences such as coding sequences and control sequences denotes sequences that are not normally associated with a region of a recombinant construct, and/or are not normally associated with a particular cell. Thus, a “heterologous” region of a nucleic acid construct can be an identifiable segment of nucleic acid within or attached to another nucleic acid molecule that is not found in association with the other molecule in nature. For example, a heterologous region of a construct can include a coding sequence flanked by sequences not found in association with the coding sequence in nature. Similarly, a host cell transformed with a construct, which is not normally present in the host cell, would be considered heterologous (see, e.g., U.S. Pat. Nos. 5,712,146 6,558,942, 6,627,427, 5,849,541 the contents of which are herein incorporated by reference). For instance, a construct with Lov genes can be isolated and expressed in non-lovastatin producing fungi or yeast host cells, and lovastatin can thereby be produced (see, e.g., U.S. Pat. Nos. 6,391,583 and 6,943,017, the contents of which are herein incorporated by reference). As another example, prokaryotes such as bacteria can be host cells also, as is known in the art. Fungal genes may also be cloned into an expression vector for expression in prokaryotes (see, e.g., U.S. Pat. No. 5,849,541, the contents of which are herein incorporated by reference).

A prokaryote such as E. coli can be used as a heterologous host. A plasmid can be constructed with a gene of interest and the plasmid can be transformed into E. coli. The gene of interest can be translated and the protein derived from the gene of interest can be purified thereafter. This method of expression and protein purification is known in the art. For example, LovD exons from A. terreus can be individually amplified from the genomic DNA of A. terreus and spliced to yield a continuous open reading frame using splice overlap extension PCR. Restriction sites can be introduced, and the gene cassette can be ligated to a vector to yield an expression construct that can be transformed into E. coli. Thereby, E. coli can be used as a heterologous host for expression of A. terreus genes. E. coli can be co-cultured with another strain that produces another substrate of interest. Additionally, substrates can be added to this culture or co-culture. Heterologous expression of the lovastatin biosynthesis genes is known in the art (see, e.g., U.S. Pat. Nos. 6,391,583 and 6,943,017, the contents of which are herein incorporated by reference).

As another example, certain polyketides, such as polyketides from fungi, or other organisms, can be heterologously expressed in E. coli, yeast, and other host organisms. These host organisms can be supplemented with other substrates, since they can require both the heterologous expression of a desired PKS and also the enzymes that produce at least some of the substrate molecules required by the PKS (see, e.g., U.S. Pat. No. 7,011,959, the contents of which are herein incorporated by reference). Similarly, fungal Lov genes can be expressed in E. coli or other bacterium, and these host bacteria can be supplemented with other substrates, such as acyl-SNAC or other acyl donor groups. These acyl donor groups can be cell permeable, and enter the bacterial cell.

“Expression vector” refers to a nucleic acid that can be introduced into a host cell to express a protein. As is known in the art, an expression vector can be maintained permanently or transiently in a cell, whether as part of the chromosomal or other DNA in the cell or in any cellular compartment, such as a replicating vector in the cytoplasm. An expression vector also comprises a promoter that drives expression of an RNA, which typically is translated into a polypeptide in the cell or cell extract. For example, suitable promoters for inclusion in the expression vectors of the invention include those that function in eukaryotic or prokaryotic host cells. Promoters can comprise regulatory sequences that allow for regulation of expression relative to the growth of the host cell or that cause the expression of a gene to be turned on or off in response to a chemical or physical stimulus. For E. coli and certain other bacterial host cells, promoters derived from genes for biosynthetic enzymes, antibiotic-resistance conferring enzymes, and phage proteins can be used and include, for example, the galactose, lactose (lac), maltose, tryptophan (trp), beta-lactamase (b/a), bacteriophage lambda PL, and T5 promoters. In addition, synthetic promoters, such as the tac promoter (U.S. Pat. No. 4,551,433), can also be used. For E. coli expression vectors, it is useful to include an E. coli origin of replication, such as from pUC, p1P, p1I, and pBR. For efficient translation of RNA into protein, the expression vector also typically contains a ribosome-binding site sequence positioned upstream of the start codon of the coding sequence of the gene to be expressed. Other elements, such as enhancers, secretion signal sequences, transcription termination sequences, and one or more marker genes by which host cells containing the vector can be identified and/or selected, may also be present in an expression vector. Selectable markers, i.e., genes that confer antibiotic resistance or sensitivity, can be used and confer a selectable phenotype on transformed cells when the cells are grown in an appropriate selective medium. For example, an expression vector containing the Lov gene cluster or portions thereof can be introduced into a heterologous host, such as E. coli. Thus, recombinant expression vectors can contain at least one expression system, which, in turn, can be composed of at least a portion of Lov and/or other biosynthetic gene coding sequences operably linked to a promoter and optionally termination sequences that operate to effect expression of the coding sequence in compatible host cells.

A “coding sequence” can be a sequence which “encodes” a particular gene, such as a gene from the Lov gene cluster, for example. A coding sequence is a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A transcription termination sequence will usually be located 3′ to the coding sequence.

DNA “control sequences” refer collectively to promoter sequences, ribosome binding sites, polyadenylation signals, transcription termination sequences, upstream regulatory domains, enhancers, and the like, which collectively provide for the transcription and translation of a coding sequence in a host cell.

“Operably linked” refers to an arrangement of elements wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof.

“Lovastatin-producing organism” refers to the wide variety of different organisms known in the art to produce lovastatin. These organisms that produce lovastatin can modified to produce simvastatin by the methods of this invention. A. terreus is an example of a lovastatin producing organism. Microorganisms other than A. terreus reported to produce lovastatin (mevinolin) include Monascus species, for example M. ruber, M. purpureus, M. pilosus, M. vitreus, M. pubigerus, as well as Penicillium, Hypomyces, Doratomyces, Phoma, Eupenicillium, Gymnoascus, and Trichoderma species, Pichia labacensis, Candida cariosilognicola, Aspergillus oryzea, Doratomyces stemonitis, Paecilomyces virioti, Penicillum citrinum, Penicillin chrysogenum, Scopulariopsis brevicaulis and Trichoderma viride (see, e.g., U.S. Pat. No. 6,391,583; Juzlova et al., J. Ind. Microbiol. 16:163-170; Gunde-Cimerman et al., FEMS Microbiol. Lett. 132:39-43 (1995); and Shindia et al., Folio Microbiol. 42:477-480 (1997), the contents of which are herein incorporated by reference).

“Non-lovastatin-producing organisms” as used herein refers to a number organisms that do not produce lovastatin absent manipulation by man (e.g. E. Coli). These organisms can be induced to produce LovD, or cultured in the presence of LovD to produce lovastatin or simvastatin by the methods of this invention, for example.

“A. terreus having a disruption in the LDKS gene” comprises an A. terreus without the LDKS gene, having a LDKS gene that is mutated, having a LDKS gene that is knocked-out, having a LDKS gene that is deleted, having a LDKS gene whose expression is disrupted, or having a LDKS gene that is disrupted. “A. terreus having a disruption in the LDKS gene” comprises an A. terreus having a LDKS gene that is silenced by methods known in the art. “A. terreus having a disruption in the LDKS gene” refers to an A. terreus that cannot produce functional LDKS. “A. terreus having a disruption in the LDKS gene” can also refer to an A. terreus that produces functional LDKS. The LDKS can be inactivated or inhibited by methods known in the art such as gene knock out protocols. The amount of LDKS present can be reduced by methods known in the art. Other methods of inhibition, inactivation, or disruption of LDKS gene or protein include, but or not limited to, antisense, siRNA, RNAi, or RNA interference as is known in the art. “LDKS gene” as used herein can also refer to the LovF gene. Disruption of the LovF gene is known in the art (see, e.g., U.S. Pat. No. 6,391,583 the contents of which are herein incorporated by reference. “A. terreus having a disruption in the LDKS gene” is typically a genetically manipulated organism. Genetic manipulation of A. terreus is known in the art. Gene disruption of the Lov genes in A. terreus has been done previously (see, e.g., U.S. Pat. Nos. 6,391,583 and 6,943,017, the contents of which are herein incorporated by reference). Disruption of specifically the LovF gene (producing LDKS) in A. terreus has been done previously (see, e.g., U.S. Pat. No. 6,943,017, the contents of which are herein incorporated by reference). Disruption of the LovF gene can occur by other methods as is known in the art. A. terreus having a disruption in the LDKS gene can be in a fermentation mixture. Substrates can be added to the fermentation mixture of an A. terreus having a disruption in the LDKS gene to produce lovastatin analogs.

“A component or method to increase the production of simvastatin” as used herein refers to a compound or substrate, synthetic or natural, that increases the production of certain intermediaries to increase the amount of simvastatin produced for scale-up and large-scale synthesis of simvastatin. Components and methods for increasing the production of certain intermediaries are known in the art. For example, compounds that are added to the fermentation mixture to increase the amount of intermediaries, such as monacolin J, in the production of lovastatin are known in the art (see, e.g., U.S. Pat. No. 6,943,017, the contents of which are herein incorporated by reference). Some of these intermediaries, such as monacolin J, can also be used in the production of simvastatin. Compounds for increasing the production of monacolin J thereby can be added to increase the production of simvastatin. For example, compounds for increasing the production of monacolin J can be directly added to the fermentation mixture to increase the amount of simvastatin produced. An example of a component for increasing the production of simvastatin is a clone containing the D4B segment of the lovastatin producing gene cluster that is deposited in ATCC accession number 98876. This clone can be transformed into a non-lovastatin producing organism to produce monacolin J as is known in the art. This clone can also be transformed into a lovastatin-producing organism to increase the production of monacolin J and thereby increase the production of simvastatin. Moreover, another example of a component for increasing the production of simvastatin is the LovE/zinc finger gene, which can be transformed into a lovastatin-producing organism to increase the production of simvastatin. Preferably, this lovastatin-producing organism would have a disruption in the LDKS gene (see, e.g., U.S. Pat. No. 6,391,583, the contents of which are herein incorporated by reference). Components and methods to increase the production of simvastatin can refer to many others and are not limited to the examples listed above.

As disclosed herein, an “Acyl donor” or “acyl carrier” is a compound having an acyl group that can be transferred to simvastatin and/or a simvastatin precursor or a related compound. Typically, “Acyl donor” or “acyl carrier” is an acyl thioester that donates an acyl moiety to the C8 hydroxyl group of monacolin J. A wide variety of such agents are known in the art that are further shown herein to have this activity (see, e.g. the illustrative acyl-thioesters in Table 1 of U.S. Pat. No. 8,211,664). In addition to those known in the art and further shown by the instant disclosure to have this activity, any potential acyl donor/carrier known in the art (or synthesized de novo) having an ability to acylate C8 of monacolin J so as to produce simvastatin can be easily identified by comparative experiments with the acyl donors disclosed herein (e.g. acyl-SNAC). As is known in the art, an acyl group can have the formula RCON; wherein R can be an alkyl or aryl and N can be —Cl, —OOCR, —NH2, —OR, or the like. Compounds that have an acyl group includes, but is not limited to, acid chlorides, esters, amides, or anhydrides and the like. These compounds can be aliphatic or aromatic, substituted or unsubstituted. Examples include, but are not limited to, benzoyl chloride, benzoic anhydride, benzamide, or ethyl benzoate, and the like. Other examples of acyl donors include, but are not limited to, α-dimethylbutyrl-SNAC, acyl-thioesters, acyl-CoA, butyryl-CoA, benzoyl-CoA, acetoacetyl-CoA, β-hydroxylbutyryl-CoA, malonyl-CoA, palmitoyal-CoA, butyryl-thioesters, N-acetylcyteamine thioesters (SNAC), methyl-thioglycolate (SMTG), benzoyl-SNAC, benzoyl-SMTG, or α-S-methylbutyryl-SNAC. These compounds can be produced naturally or synthetically, and, in some cases, can penetrate the cell membrane. A number of these compounds can be added to LovD in the presence of monacolin J to produce simvastatin for example.

“Acyl-SNAC” as used herein refers to α-dimethylbutyrl-SNAC. As is known in the art, acyl-SNAC can penetrate the cell membrane under in vivo conditions. LovD can use acyl-SNAC as a substrate to initiate the reaction from monacolin J to simvastatin by regiospecifically acylating the C8 hydroxyl group of monacolin J. Acyl-SNAC can donate its acyl group to LovD.

Lanza et al., A condition-specific codon optimization approach for improved heterologous gene expression in Saccharomyces cerevisiae BMC Syst Biol. 2014 Mar. 17; 8:33. doi: 10.1186/1752-0509-8-33.

Well-known methods for improving heterologous expression include codon-optimization of the heterologous nucleotide sequence. This is done by employing the host-preferred codons, as determined from codons of the highest frequency in highly expressed proteins of the host of interest. The coding sequence for a polypeptide having PUFA desaturase or elongase activity can be chemically synthesized in whole or in part using methods well established in the literature. Moreover, the nucleotide sequence surrounding the translational start-codon ATG has been found to influence gene expression in yeast. If the desired polypeptide is poorly expressed in yeast, the nucleotide sequence of the heterologous gene can be modified to include an efficient yeast translation initiation sequence to obtain optimal gene expression. This can be accomplished by standard techniques such as PCR-based site directed mutagenesis or by fusion to the initiation sequence of a highly expressed yeast gene.

### Typical Embodiments of the Invention

Those of skill in the art will understand that the disclosure provided herein allows artisans to produce a wide variety of embodiments of the invention. The present invention provides systems and methods of biosynthesizing simvastatin directly from an engineered microbial host using an engineered biosynthetic pathway (such as Saccharomyces cerevisiae). Simvastatin can be obtained by feeding a simple synthetic building block to a fermentated culture. This system and method can be a cost-effective way of obtaining simvastatin.

Furthermore, according to various aspects of the present invention, purification of lovastatin or monacolin J can also be skipped. The engineered Saccharomyces cerevisiae is able to produce monacolin J by its own and can synthesize simvastatin when a simple 2,2-dimethylbutyryl donor is provided to the same culture. This is a great simplification to the current procedures known in the art for producing this semi-synthetic natural product.

The present invention takes advantage of the knowledge regarding lovastatin biosynthesis and a well-developed fungal PKS expression platform in Saccharomyces cerevisiae. Generally, lovastatin is biosynthesized in a fungus Aspergillus terreus as follows: 1) The lovastatin nonaketide synthase (LNKS or LovB), together with the dissociated enoylreductase LovC, are responsible for the programmed assembly of dihydromonacolin L (DML). 2) As described in detail in the Example 2, a trans-acting TE (LovG) is found to be required to efficiently release DML acid from LovB. 3) The resulting DML acid is then oxidized by the cytochrome P450 monooxygenase LovA via hydroxylation/dehydration steps to afford monacolin J acid. 4) The α-methylbutyryl side chain is synthesized by lovastatin diketide synthase (LDKS or LovF). 5) This α-methylbutyryl side chain is transferred to the C-8 hydroxyl group of monacolin J acid by the acyl transferase LovD to yield the acid form of lovastatin. The same enzyme, LovD, is also able to efficiently transfer 2,2-dimethylbutyryl to C-8 hydroxyl group of monacolin J and produce simvastatin, when a proper acyl donor (like 2,2-dimethylbutyryl-SMMP) is provided.

According to one or more aspects of the invention, an engineered S. cerevisiae strain is able to heterologously express functional fungal PKSs. Both PKS proteins (LovB and LovF) may be reconstituted from this strain. The P450, LovA, may also be functionally reconstituted in S. cerevisia. By expressing all the necessary lovastatin genes (lovA, lovB, lovC, lovD and lovG, including the cytochrome P450 oxidoreductase (CPR)) in yeast and feeding the 2,2-dimethylbutyryl donor, simvastatin may be directly produced from this S. cerevisiae strain.

In one or more embodiments of the invention, all five of the lov enzymes are expressed episomally from three separate vectors. The titer of simvastatin can be significantly improved with additional metabolic engineering approaches. This invention can lead to several important, new methods of producing simvastatin that is advantageous over existing technology, including: 1) single step fermentation of S. cerevisiae to yield simvastatin; and 2) co-culturing of the monacolin J producing S. cerevisiae with a LovD over-express strain for improved conversion from monacolin J to simvastatin. In each case, the acyl substrate dimethylbutyryl-S-methylmercaptopropionate can be synthesized chemically and be added to the fermentation broth.

The titer of simvastatin in S. cerevisiae may be further improved through: 1) integration of these genes into chromosomal DNA of S. cerevisiae to ensure steady production; 2) engineering the S. cerevisiae malonyl-CoA synthetic pathway to provide more building blocks and improve efficiency of simvastatin biosynthesis; and 3) development of techniques to display of LovD on surface of S. cerevisiae for further simplification of product purification.

As discussed in detail below, the isolated organism can be grown under one of a variety of fermentation conditions known in the art and the exact conditions are selected, for example based upon fermentation parameters associated with optimized growth of a specific organism used in an embodiment of the invention (see, e.g. Miyake et al., Biosci. Biotechnol. Biochem., 70(5): 1154-1159 (2006) and Hajjaj et al., Applied and Environmental Microbiology, 67: 2596-2602 (2001), the contents of which are incorporated by reference). Typically, the organism is grown at a temperature between 30-40° C., for a time period between at least 4 to at least 48 hours. Typically, the organisms are grown at a pH between 6.5-8.5. In certain embodiments of the invention, the pH of the fermentation media can be 6.9, 7.0, 7.1, 7.2, 7.3, 7.4, 7.5, 7.6, 7.7, 7.8, 7.9, 8.0 or 8.1. In illustrative embodiments, the organism is grown in a fermentation media comprising LB, Fl or TB (e.g. for E. coli) or YPD, YNB, HC or YC, (e.g. for yeast) media.

Certain embodiments of the methods for making simvastatin include further steps to purify simvastatin by the combination. For example, some embodiments of the invention include at least one purification step comprising lysis of cells of an isolated organism present in the combination. Embodiments can include at least one purification step comprising centrifugation of cells or cell lysates of an isolated organism present in the combination. Embodiments can include at least one purification step comprising precipitation of one or more compounds present in the combination. Embodiments can include at least one purification step comprising filtration of one or more compounds present in the combination. Embodiments can include at least one purification step comprising a high performance liquid chromatography (HPLC) analysis of one or more compounds present in the combination.

A variety of acyl thioesters that can be used in the compositions of the invention are disclosed herein. Typically, the acyl thioester is a butyrlyl-thioester, a N-acetylcysteamine thioester or a methyl-thioglycolate thioester. Optionally, the acyl thioester comprises medium chain length (C3-C6) acyl group moieties. In certain embodiments of the invention, the acyl thioester is able to cross the cellular membranes of Escherichia coli or Aspergillus terreus cells growing within a fermentation media. Typically, the acyl thioester is selected from the group consisting of α-dimethylbutyryl-S-methyl-mercaptopropionate (DMB-S-MMP), dimethylbutyryl-S-ethyl mercaptopropionate (DMB-S-EMP) and dimethylbutyryl-S-methyl thioglycolate (DMB-S-MTG) and dimethylbutyryl-S-methyl mercaptobutyrate (DMB-S-MMB). In an illustrative embodiment, the acyl thioester is α-dimethylbutyryl-S-methyl-mercaptopropionate that is combined in fermentation media in a concentration range of 1 mM-100 mM and can typically be about 10, 20, 30, 40, 50, 60, 70, 80 or 90 mM. In some embodiments of the invention, the composition further comprises lovastatin and the amount of simvastatin in the composition is greater than the amount of lovastatin in the composition.

As disclosed herein, an “acyl thioester to regioselectively acylate the C8 hydroxyl group of monacolin J” is a compound having an acyl group that can be transferred to monacolin J or a related compound so as to make simvastatin or a related compound as disclosed herein. A wide variety of such agents are known in the art that are further shown herein to have this activity (see, e.g. the illustrative acyl-thioesters in Table 1 of U.S. Pat. No. 8,211,664). In addition to those known in the art and further shown by the instant disclosure to have this activity, any potential acyl donor/carrier known in the art (or synthesized de novo) that further has an ability to acylate C8 of monacolin J so as to produce simvastatin can be easily identified by comparative experiments with the acyl donors disclosed herein (e.g. acyl-SNAC). Typically in such experiments, the acyl thioester is a butyrlyl-thioester, a N-acetylcysteamine thioester or a methyl-thioglycolate thioester. Optionally, the acyl thioester comprises medium chain length (C3-C6) acyl group moieties. In certain embodiments of the invention, the acyl thioester is able to cross the cellular membranes of Escherichia coli or Aspergillus terreus cells growing within a fermentation media. Typically, the acyl thioester is selected from the group consisting of α-dimethylbutyryl-S-methyl-mercaptopropionate (DMB-S-MMP), dimethylbutyryl-S-ethyl mercaptopropionate (DMB-S-EMP) and dimethylbutyryl-S-methyl thioglycolate (DMB-S-MTG) and dimethylbutyryl-S-methyl mercaptobutyrate (DMB-S-MMB).

### EXAMPLES

The Examples below provide illustrative methods and materials that can be used in the practice of the invention.

### Example 1

### Direct Production of Simvastatin from Yeast

In one example, in vivo production of dihydromonacolin L in yeast was first examined. Three enzymes (LovB, LovC and LovG) were expressed episomally from three separate vectors and DML was produced at titer of ˜35 mg/L. Monacolin J was then produced from BJ5464-NpgA through introduction of LovA and CPR. ADH2p-lovG-ADH2t cassette was cloned into the same expression vector for lovC. LovA and CPR gene were later co-expressed from the third vector and monacolin J was observed to be produced at titer of ˜20 mg/L. Lastly, lovD gene was codon optimized and this gene was introduced through cloning the ADH2p-lovD-ADH2t cassette into the vector for lovC and lovG. Then all the five enzymes from these three vectors were co-expressed and the strain was fed with the acyl donor dimethylbutyryl-S-methylmercaptopropionate. Direct production of simvastatin from yeast was observed.

### Example 2

### Requirement of LovG for Dihydromonacolin L Release and Lovastatin Nonaketide Synthase Turnover

Lovastatin, a polyketide produced by the fungus Aspergillus terreus [1], is one of the most important natural products discovered to date. Both lovastatin and the semi-synthetic derivative simvastatin are widely prescribed hypercholesterolemia drugs because of their inhibitory activities towards 3S-hydroxy-3-methylglutaryl-CoA reductase (HMGR) [2]. The fermentative production of lovastatin has therefore been one of highest grossing processes involving a natural product. We recently developed a biocatalytic process of converting an intermediate of the lovastatin biosynthetic pathway, monacolin J acid (3) into simvastatin acid (5) in a single enzymatic step [3]. Therefore, having a fast-growing heterologous host, such as Saccharomyces cerevisiae, that can produce monacolin J acid (3) directly without the need to purify lovastatin followed by side chain hydrolysis, is an attractive process option. To do so, identification of a complete biosynthesis pathway leading to monacolin J acid (3) is required.

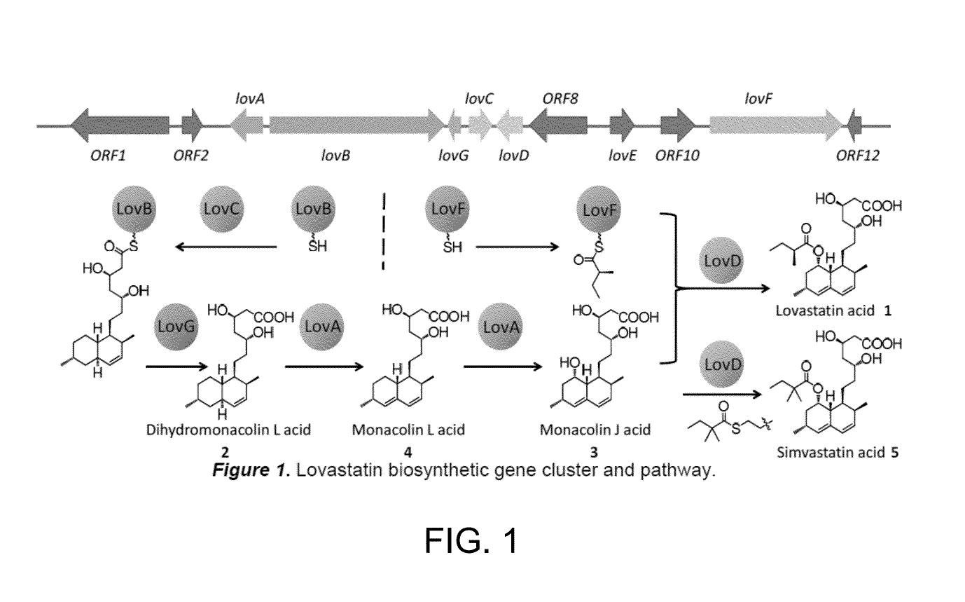

The biosynthetic gene cluster for lovastatin acid (1) in A. terreus was first identified in a pioneering work by Kennedy et al [4]. Through genetic [4-5] and biochemical [6] characterizations, it is now known that two highly reducing iterative type I polyketide synthases (HR-PKSs) play central roles in biosynthesis of lovastatin acid (1). The lovastatin nonaketide synthase (LNKS or LovB), together with the dissociated enoylreductase LovC, are responsible for the programmed assembly of dihydromonacolin L acid (2) [4]. Dihydromonacolin L acid (2) is then oxidized by the cytochrome P450 monooxygenase LovA via multiple hydroxylation steps to first afford monacolin L acid (4) followed by monacolin J acid (3) [6c]. Lastly, the α-methylbutyryl side chain is synthesized by lovastatin diketide synthase (LDKS or LovF) and is transferred to the C-8 hydroxyl group of monacolin J acid (3) by the acyl transferase LovD to yield lovastatin acid (1) [6a] (FIG. 1). It is the function of LovD that was exploited for the enzymatic synthesis of simvastatin [7].

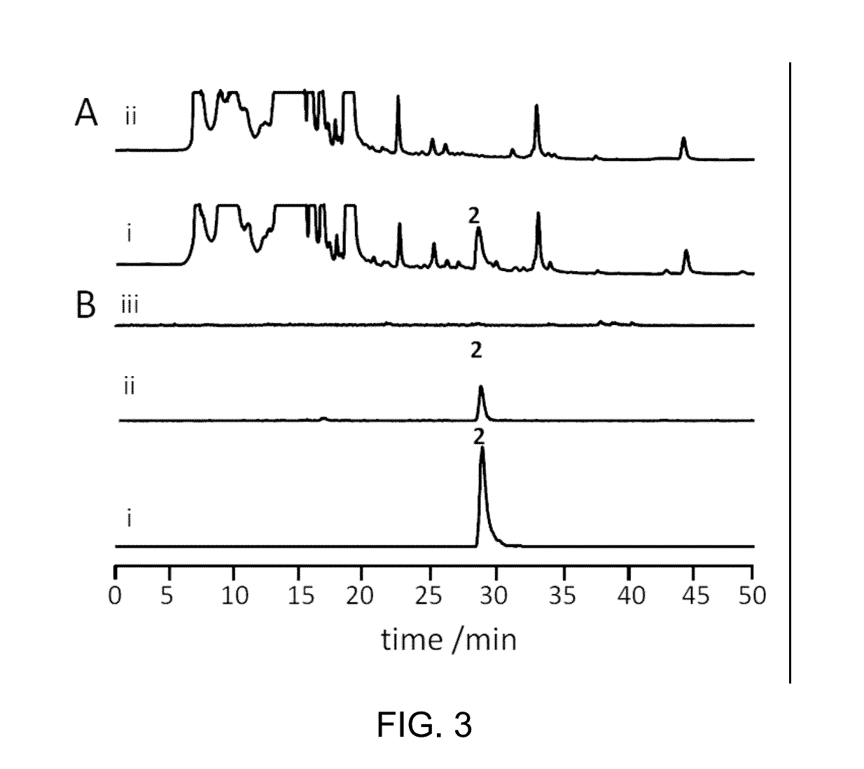

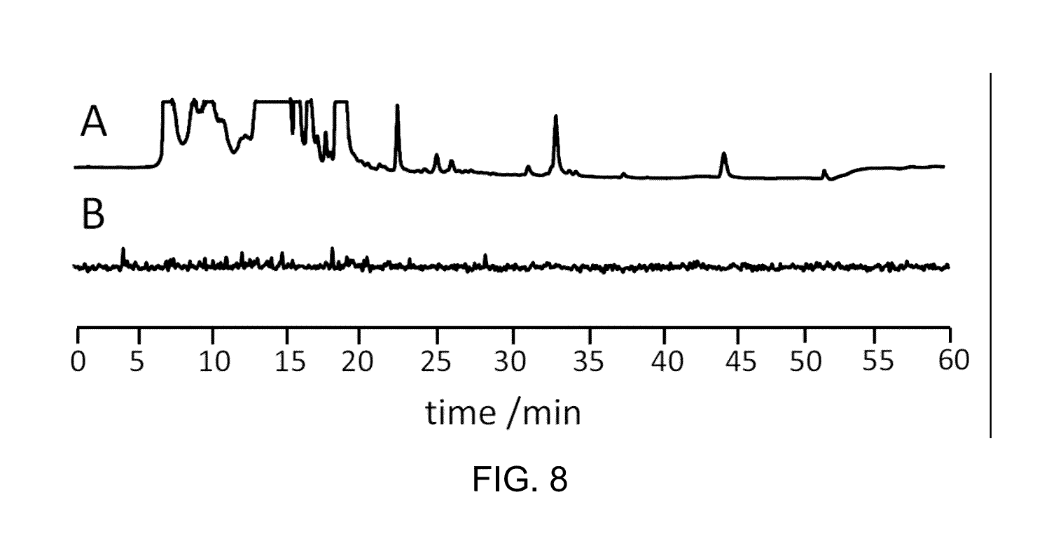

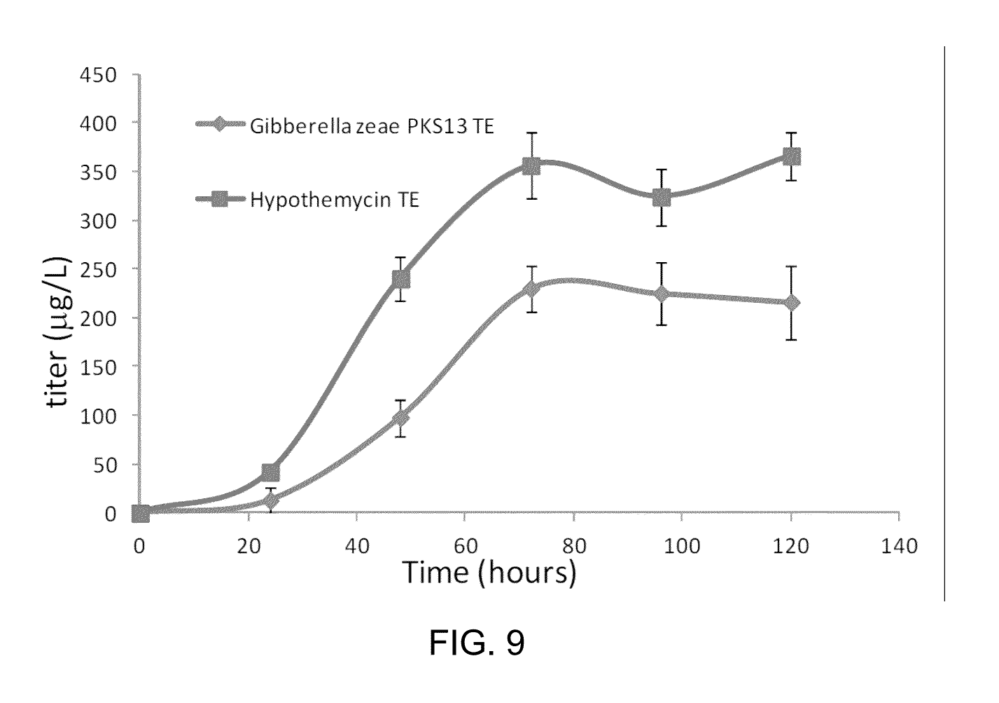

Notwithstanding these insights into the lov pathway, one unsolved biochemical step is the release of dihydromonacolin L acid (2) from LovB that allows multiple turnover by this enzyme. In our previous in vitro reconstitution work, we showed that purified LovB and LovC were sufficient to assemble dihydromonacolin L acid (2) tethered to LovB from malonyl-CoA, but were not able to release the product [6b]. To test whether this is also the case in S. cerevisiae, LovB and LovC were coexpressed in S. cerevisiae BJ5464-NpgA, which is a vacuolar protease-deficient yeast strain harbouring an A. nidulans phosphopantethienyl transferase npgA [8]. Following extraction of the 3-day yeast culture, no trace of dihydromonacolin L acid (2) was found by selective ion monitoring (FIG. 3A and FIG. 8B), confirming the lack of a releasing function with LovB and LovC only. Serendipitously in our in vitro work, we identified non-native thioesterase (TE) domains from unrelated fungal PKSs that can offload dihydromonacolin L acid (2) [6b]. Coexpression of fungal TE domains, such as the PKS13 TE [9], Hpm3 TE [10], etc, with LovB and LovC resulted in detectable levels of dihydromonacolin L acid (2) from BJ5464-NpgA, albeit with very low titers (<400 μg/L, FIG. 9). Given the known expression levels of LovB (˜4 mg/L) [6b], this low titer corresponds to an average turnover of less than 90 for LovB. These studies strongly indicate that 1) the low titer of dihydromonacolin L acid (2) from S. cerevisiae must be overcome by increasing the turnover rate of LovB; and 2) there must exist a natural TE that partners with LovB in the release of dihydromonacolin L acid (2).

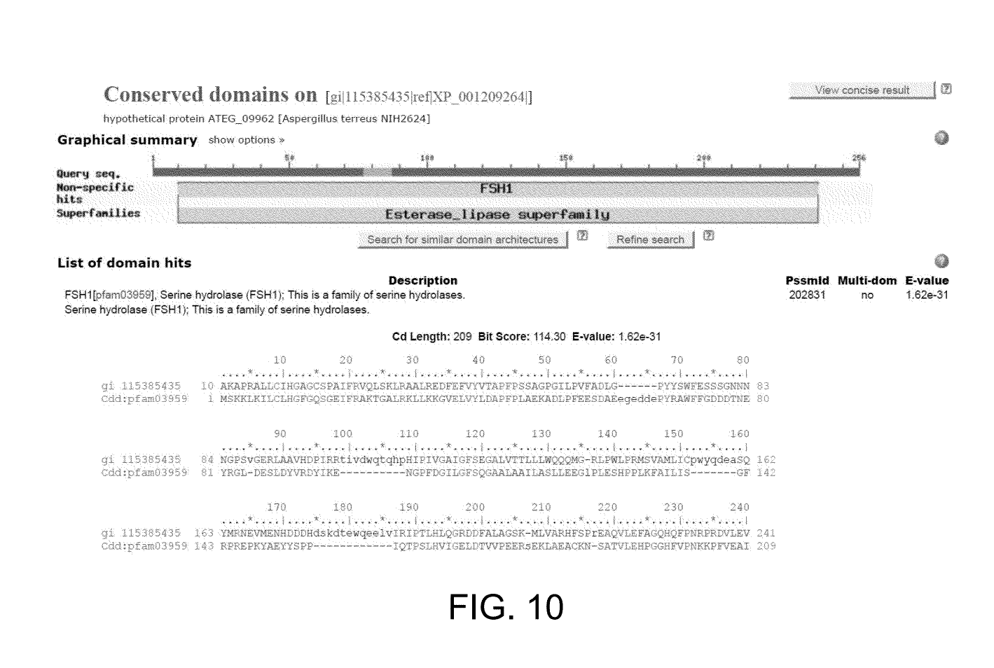

To identify the TE that is involved in the biosynthesis of dihydromonacolin L acid (2), we revisited the A. terreus lov gene cluster and searched for a likely candidate. The only known TE-like enzyme in the gene cluster, LovD, is highly specific towards LovF and does not function with LovB. A thorough bioinformatic analysis of genes of unassigned function suggests that a gene (ATEG_{—}09962) that is located between lovB and lovC (FIG. 1) warrants further scrutiny. This gene was initially assigned as orf5 [4] and later as lovG [11], with an annotation of the protein product as either a hypothetical protein or an oxidoreductase. Conserved domain analysis of LovG indicates that LovG in fact belongs to the esterase-lipase family of serine hydrolases (FIG. 10). Notably, close homologs of LovG are also found in related biosynthetic pathways, including MlcF in the compactin pathway in Penicillium citrinum [12] (58% identity, 71% similarity); and MokD in the monacolin K (lovastatin) pathway in Monascus pilosus [13] (67% identity, 76% similarity). As is the case for lovG, both mlcF and mokD are located between the genes encoding nonaketide PKS and the trans-acting enoylreductase. RTPCR analysis showed that mlcF and mokD are moderately cotranscribed with the other biosynthetic genes in their corresponding clusters, which suggests that these enzymes should be involved in the biosynthetic pathways [12-13]. Therefore, we hypothesized that LovG is the missing link that is required for LovB turnover and release of dihydromonacolin L acid (2).

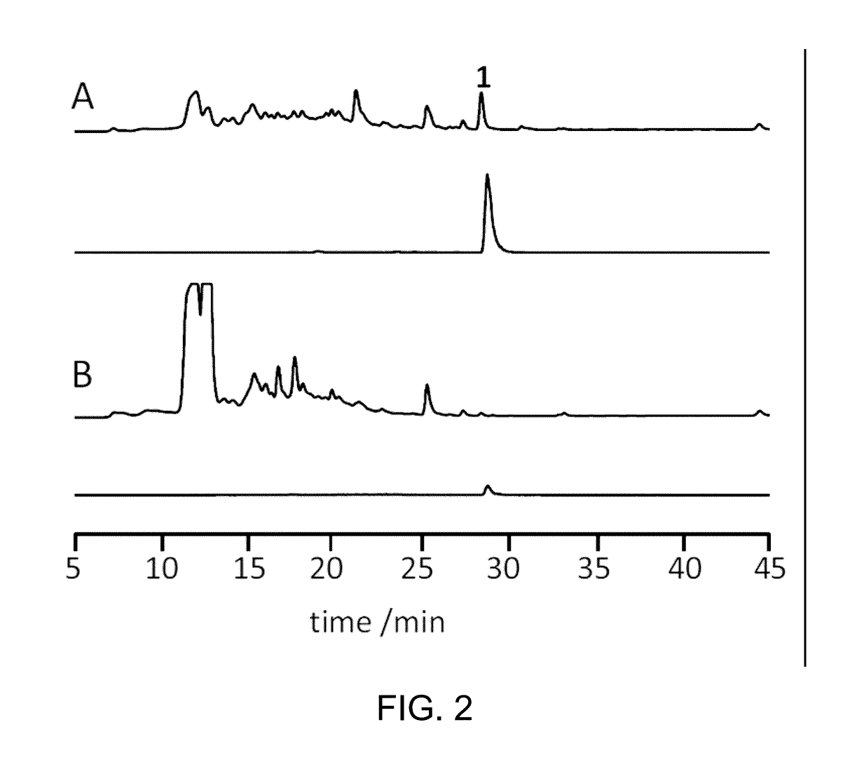

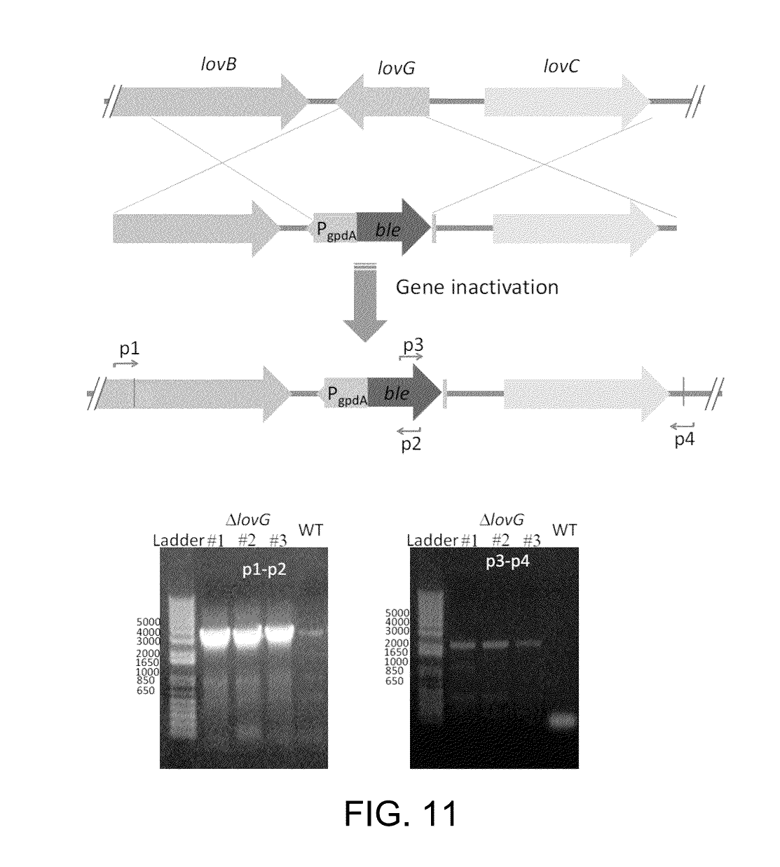

We first determined the role of lovG in biosynthesis of lovastatin acid (1) in A. terreus. A genetic disruption of lovG was performed using a double-crossover recombination with the zeocin-resistant marker, followed by identification of desired AlovG mutants via diagnostic PCR (FIG. 11). Metabolite analysis of the AlovG mutants showed dramatically reduced level of lovastatin acid (1) (<5%) compared to the wild type (FIG. 2). No intermediate such as dihydromonacolin L acid (2), monacolin L acid (4) or monacolin J acid (3) was accumulated in the culture, indicating LovG is involved in the early part of the pathway. The significant attenuation of lovastatin acid (1) biosynthesis indicates that lovG is intimately involved in the lov pathway, but not absolutely essential for the production of lovastatin acid (1). This is not unexpected given our previous findings: an endogenous TE in A. terreus may be recruited for the hydrolysis of dihydromonacolin L acid (2), albeit at a much lower efficiency. In a recent study, the region encoding lovG (between lovB and lovC) was replaced by sequences encoding the nonribosomal peptide synthetase (NRPS) module from the CheA PKS-NRPS to yield LovB fused with a NRPS module[14]. The authors observed a similar reduction in the level of lovastatin acid (1), and attributed the drop in yield to the reduced efficiency of the chimeric LovB [14]. From the results shown in FIG. 2, we believe the decrease titer of lovastatin acid (1) in that study can also be from the unintended deletion of lovG.

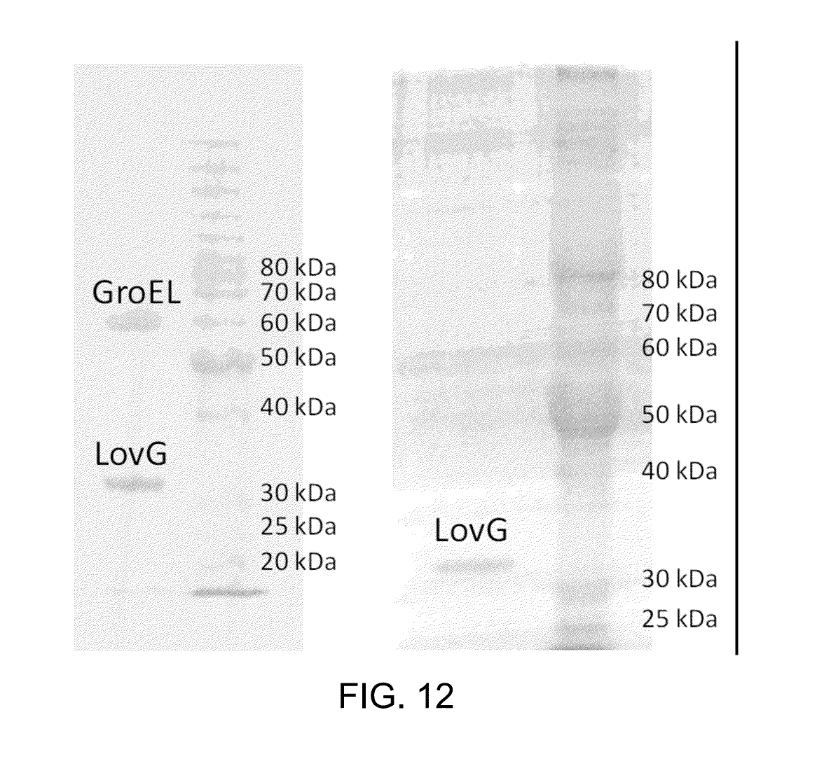

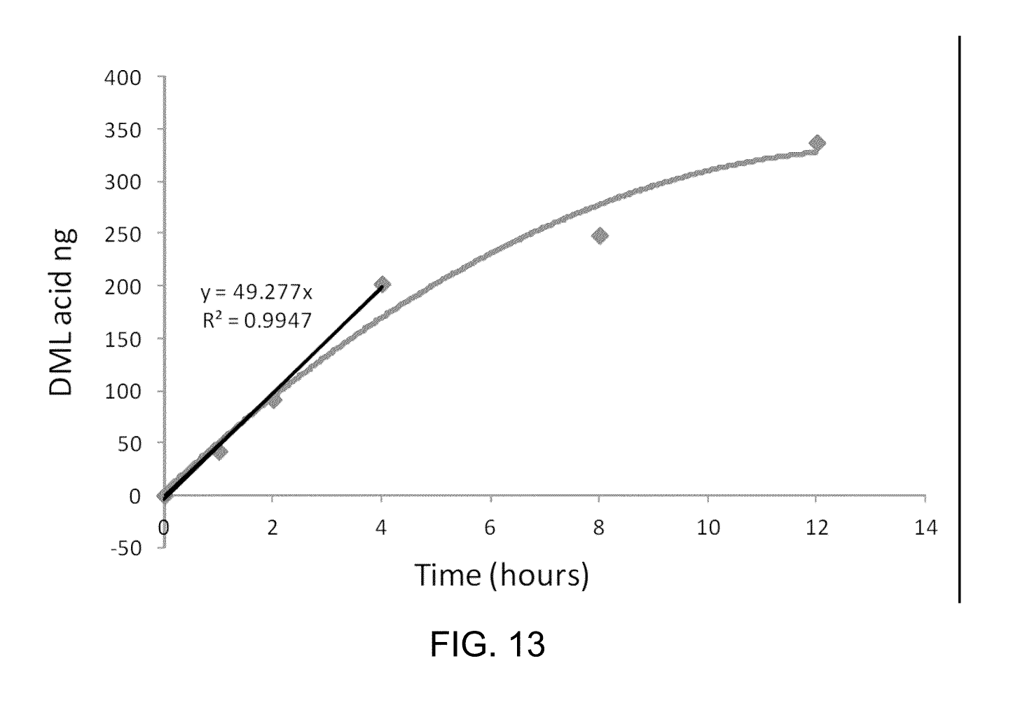

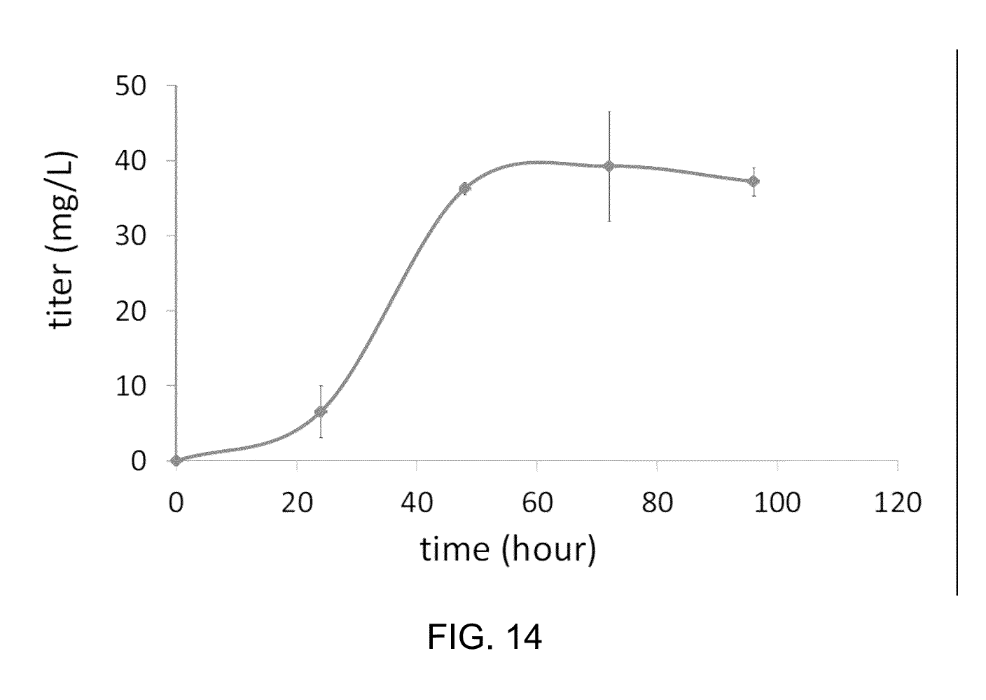

To further confirm the hydrolytic role of LovG, we probed the direct involvement of this enzyme in turnover of dihydromonacolin L acid (2) by in vitro and in vivo reconstitution assays. First, LovG was expressed and purified as N-terminal His-tagged protein from E. coli BL21(DE3). Chaperone proteins GroES/EL, DnaK/J and GrpE were coexpressed [15] to assist the folding of LovG and to improve the solubility (FIG. 12). By incubating equimolar amounts (10 μM) of purified LovB, LovC and LovG with malonyl-CoA and the necessary cofactors (NADPH, SAM) for 12 hours at 25° C., we observed production of dihydromonacolin L acid (2) in the assay without the need to add either base (1M KOH) or heterologous TE (FIG. 3B). With LovG included, the turnover rate of dihydromonacolin L acid (2) was estimated to be ˜0.12 hour⁻¹. (FIG. 13) The slow, albeit observable turnover may be due to the large fraction of recombinant LovB being inactive in the in vitro assay as previously noted [6b]. Furthermore, lovG was cloned under S. cerevisiae ADH2 promoter and coexpressed with lovB and lovC. This resulted in the production of dihydromonacolin L acid (2) from S. cerevisiae BJ5464-NpgA at an elevated titer of ˜35 mg/L after ˜50 hours of culturing (FIG. 3A, FIG. 14). Collectively, these results confirmed LovG is the natural releasing partner of LovB during lovastatin biosynthesis in A. terreus.

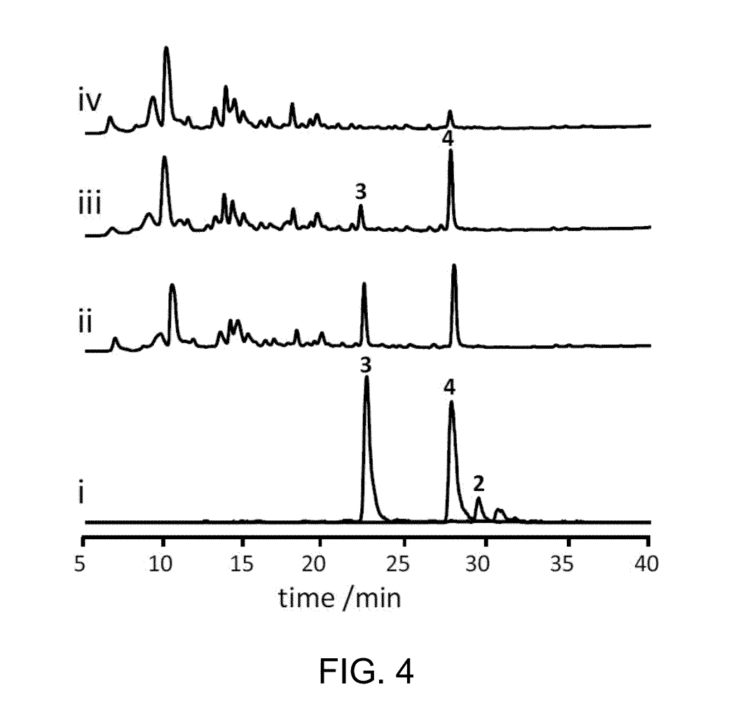

The successful production of dihydromonacolin L acid (2) following LovG coexpression prompted us to examine the ability of the yeast host to produce more advanced intermediates such as the desired monacolin J acid (3). The 2μ vector encoding both lovA and the endogenous A. terreus cytochrome P450 oxidoreductase (CPR) from A. terreus[6c] was introduced into BJ5464-NpgA and coexpressed with LovB, LovC and LovG. In the absence of expression of LovA and CPR, which are controlled by the divergent GAL1-GAL10 promoter, we were only able to observe the accumulation of dihydromonacolin L acid (2). When galactose was added to the culture media at day 2, nearly all of dihydromonacolin L acid (2) was oxidized to monacolin L acid (4) and monacolin J acid (3). After 48 hours, both monacolin J acid (3) and monacolin L acid (4) can be detected from the yeast culture at ˜20 mg/L each (FIG. 4). It is important to note here that this initial demonstration of biosynthesis of monacolin J acid (3) has not been optimized. All five of the lov enzymes were expressed episomally from three separate 2μ vectors. With additional metabolic engineering approaches, we expect the conversion from monacolin L acid (4) to monacolin J acid (3), as well as the titer of monacolin J acid (3) can be significantly improved.

LovG joins a growing list of fungal TEs that are involved in product release via either hydrolysis or acyl transfer to an onpathway intermediate (such as LovD [6a]). All of the TEs associated with HR-PKSs characterized to date are stand-alone enzymes, in contrast to the fused TE domains in mammalian fatty acid synthases (FAS) [16] or some of the nonreducing PKSs (NR-PKS), in which the fused TE can act as Claisen-like Cyclases (TE/CLC) [17]. Recently, TE/CLCs from NR-PKS was also shown to possess editing functions during the PKS function and hydrolyze stalled products [18]. Given that LovB is noted to be highly accurate in the assembly of dihydromonacolin L acid (2) as a sole product both in vitro and here, in vivo, we hypothesized that LovG may exert proofreading functions during the iterative LovB functions to remove aberrant tailored products. This function can ensure the timely offloading of incorrect, stalled intermediates and free LovB for more efficient turnover of dihydromonacolin L acid (2).

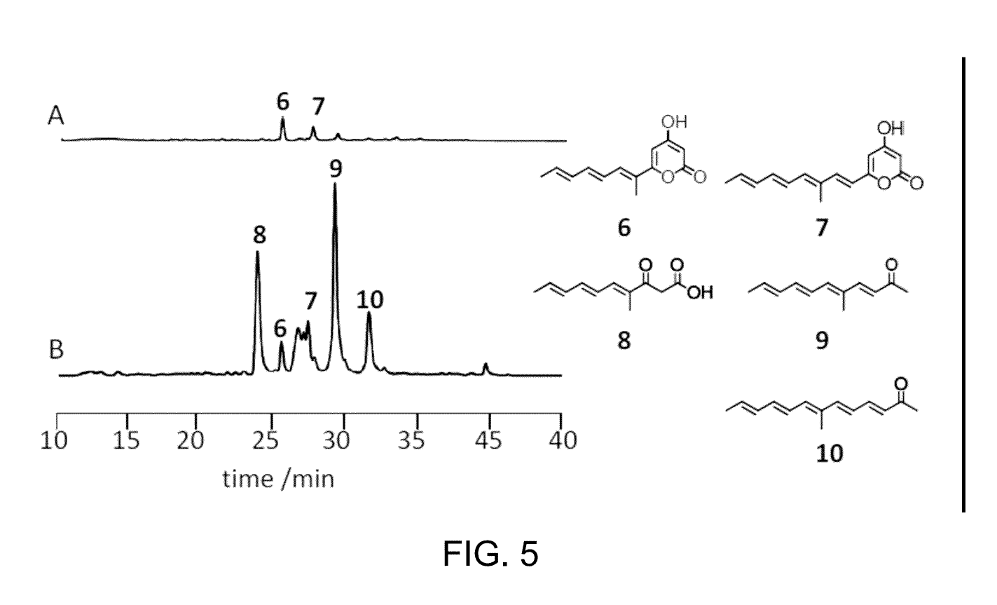

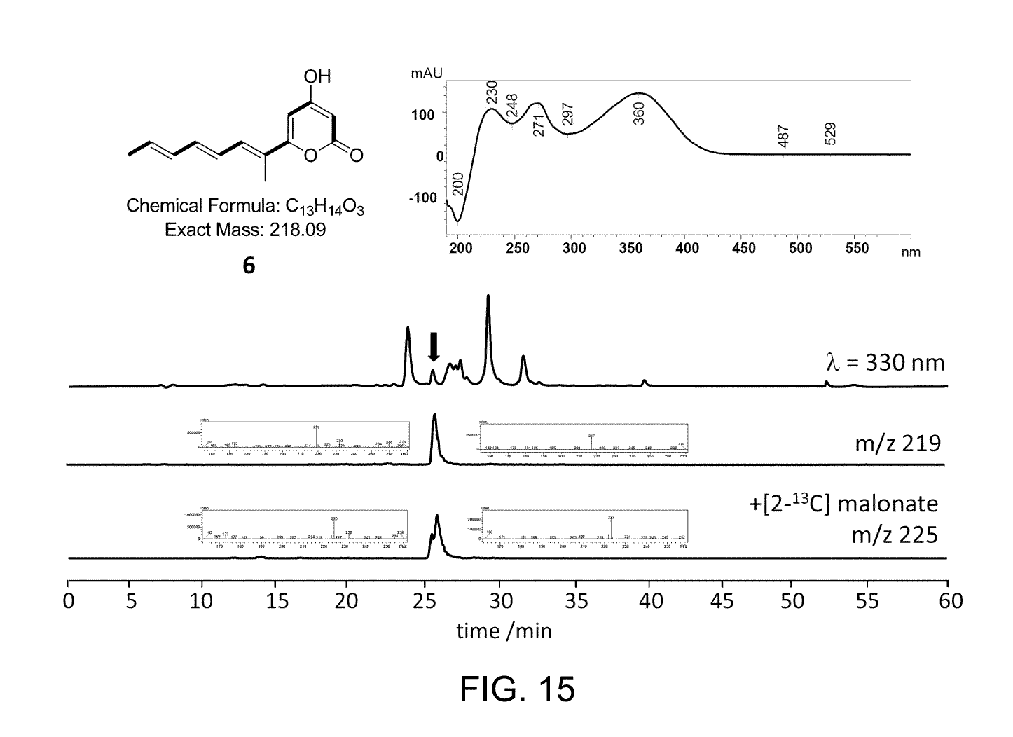

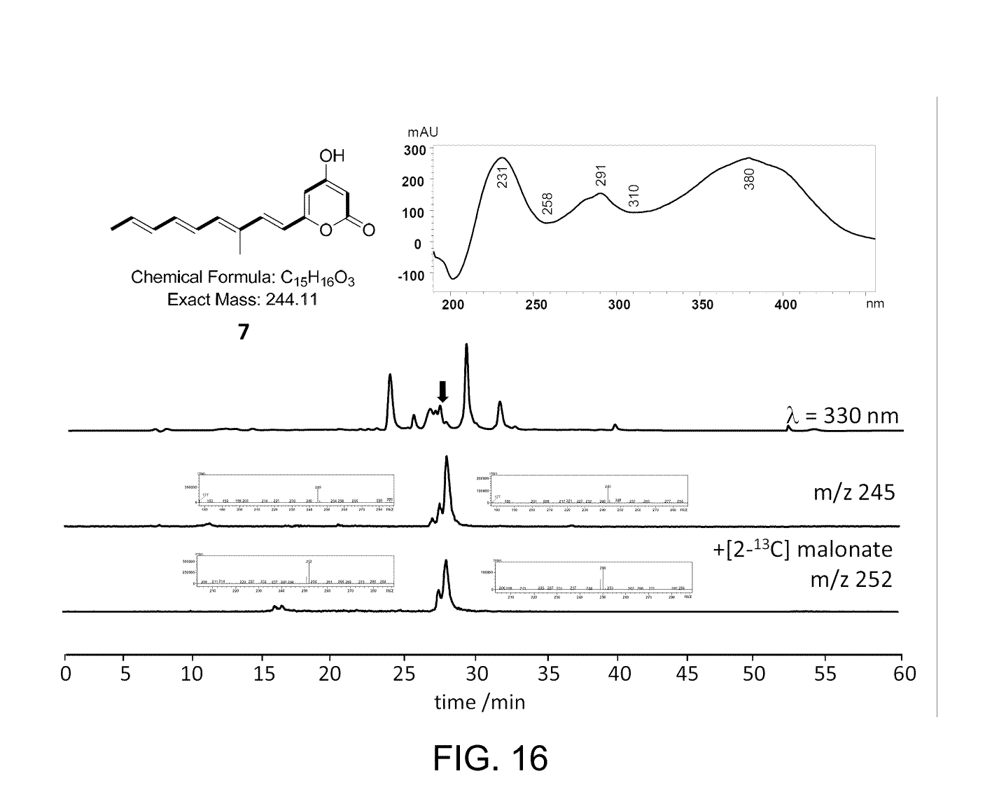

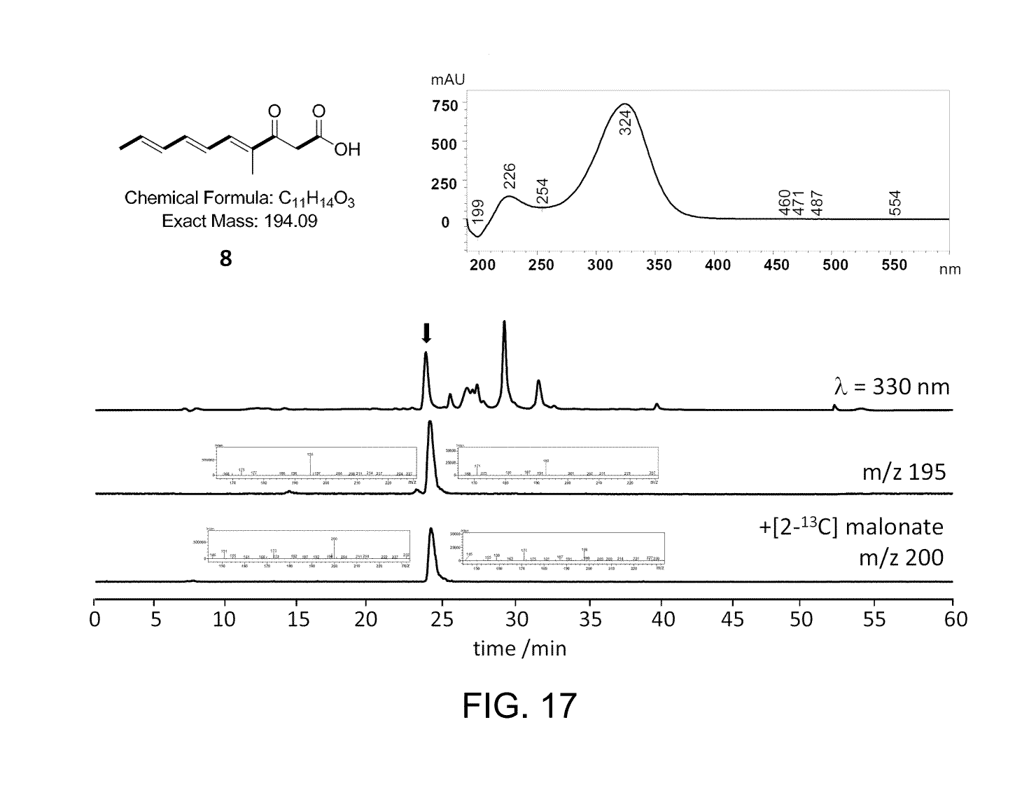

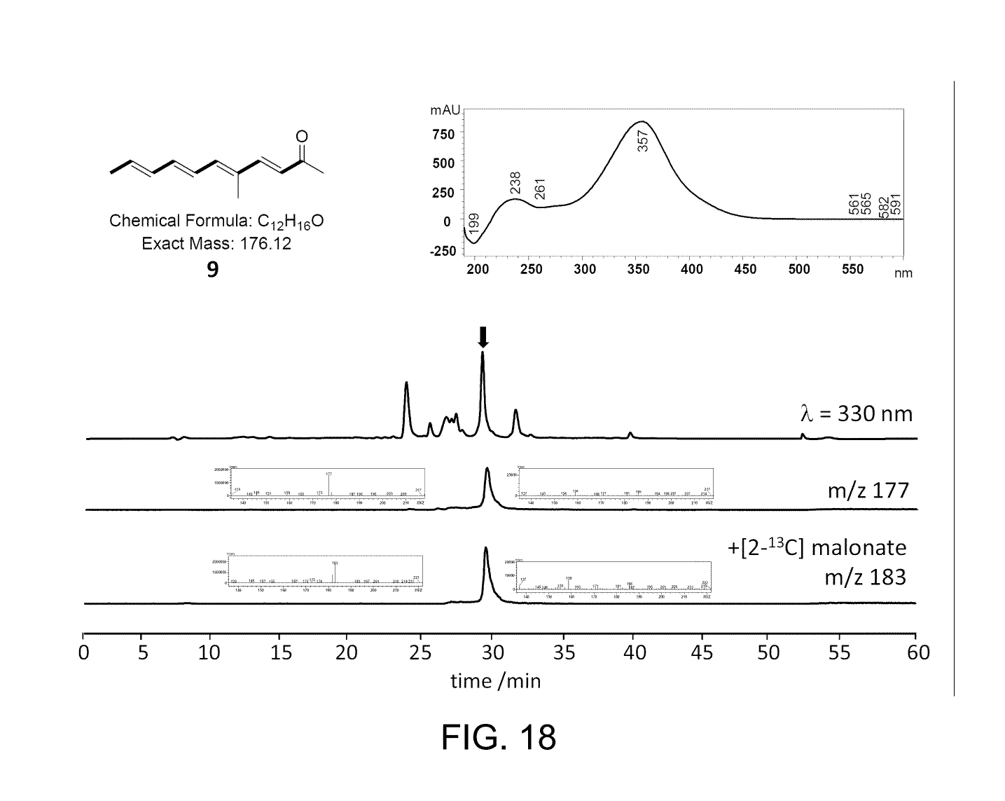

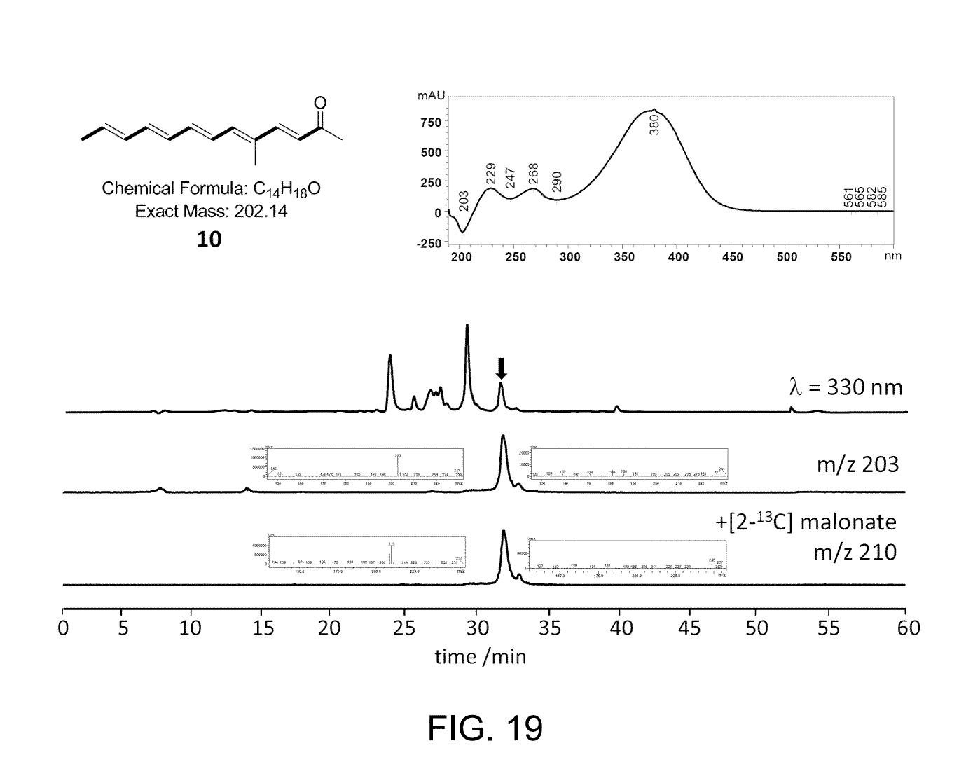

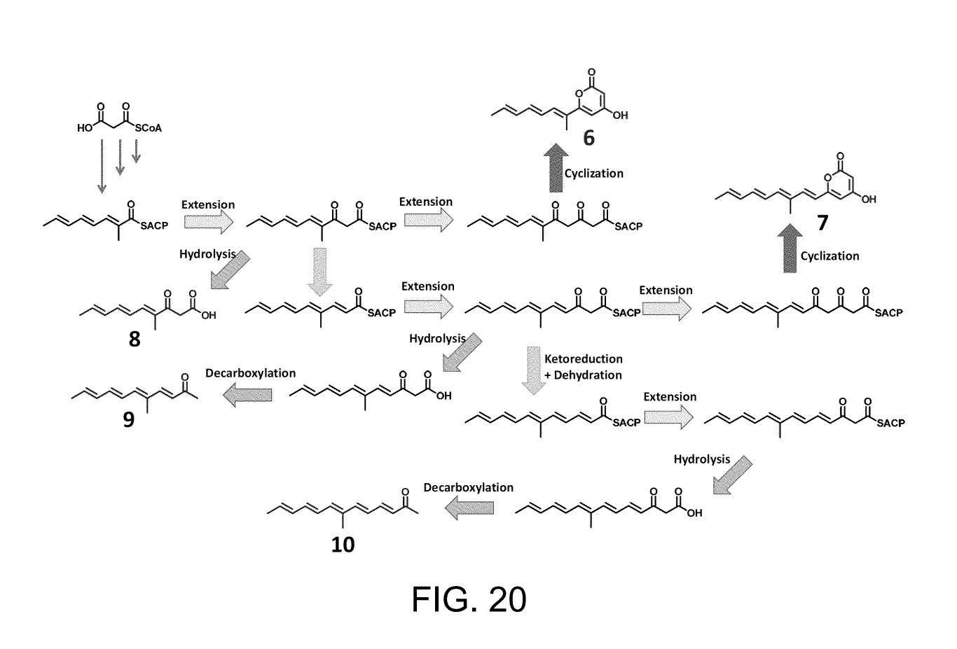

To assay the possible editing function of LovG, we performed in vitro reconstitution experiments in the absence of LovC, of which the earliest function in the pathway is to reduce the α-β enol intermediate at the tetraketide stage. We previously observed small amounts of offloaded products from LovB in the forms of methylated, conjugated α-pyrones such as 6 and 7 (FIG. 5A) via continued chain elongation and spontaneous esterification); and ketones (not visible at the scale drawn in FIG. 5A) via hydrolysis of β-keto thioesters and decarboxylation), when LovC is excluded [4,6b]. Upon addition of LovG at equimolar concentration to LovB, the in vitro assay mixture turned significantly more yellow and the extract contained high amounts of offloaded products 8-10, in addition to 6 and 7 (FIG. 5B). The compounds 8-10 were not stable and precluded the precise structural determination by NMR. However, through comparison of UV absorption spectra and mass increase upon using [2-13C] malonate (FIG. 15-19), as well as previously deciphered programming rules of LovB [6b], we were able to deduce the structures of these compounds as shown in FIG. 5. 8 is a hydrolyzed β-keto acid, while 9 and 10 are ketones resulting from the decarboxylation of longer polyene β-keto acids. Interestingly, while the levels of 8-10 are high in the LovG-containing assay, the relative amounts of pyrones 6 and 7 remained nearly the same as in assay with LovB alone, indicating that LovG can readily hydrolyze the incorrectly tailored, β-keto thioesters from LovB to give 8-10. In contrast, the spontaneous hydrolysis of β-keto intermediates is much slower without LovG, resulting in LovB having to offload the products through an additional round of chain elongation, which can then be cyclically released as 6 and 7 (FIG. 20). A recent study showed that NR-PKS TEs can also promote the formation of pyrones [19]. Therefore, LovG may also assist in the formation of 6 and 7, albeit likely at a much lower level as evident in FIG. 5.

In conclusion, we have identified LovG as a multifunctional esterase from the lovastatin gene cluster. LovG is not only involved in the release of the correct product 2 from LovB, but is also shown to play a role in the clearance of aberrant intermediates from LovB. Construction of the LovG-containing pathway capable of de novo synthesis of monacolin J acid (3) also opens up new metabolic engineering opportunities for statin production from yeast.

### Experimental Procedure

### I. General Molecular Biology

### 1. Strains and General DNA Manipulation Techniques

E. coli TOP10 (Invitrogen) and E. coli XL1-Blue (Stratagene) were used for cloning, following standard recombinant DNA techniques. DNA restriction enzymes were used as recommended by the manufacturer (New England Biolabs). PCR was performed using AccuPrime™ Pfx DNA Polymerase (Invitrogen). The constructs of pCR-Blunt vector (Invitrogen) containing desired PCR products were confirmed by DNA sequencing. E. coli BL21(DE3) (Novagen) was used for protein expression. Saccharomyces cerevisiae strain BJ5464-NpgA was used as the yeast expression host. BJ5464-NpgA was created through chromosomally integrating the A. nidulans phosphopantetheinyl transferase gene npgA to BJ5464 (MATaura3-52 his3-Δ200 leu2-Δ1 trp1 pep4::HIS3 prb1 Δ1.6R can1 GAL).[20],[8]

### 2. Plasmid Construction

### Cloning of N-his6 (SEQ ID NO: 15) LovG Expression Vector in E. coli

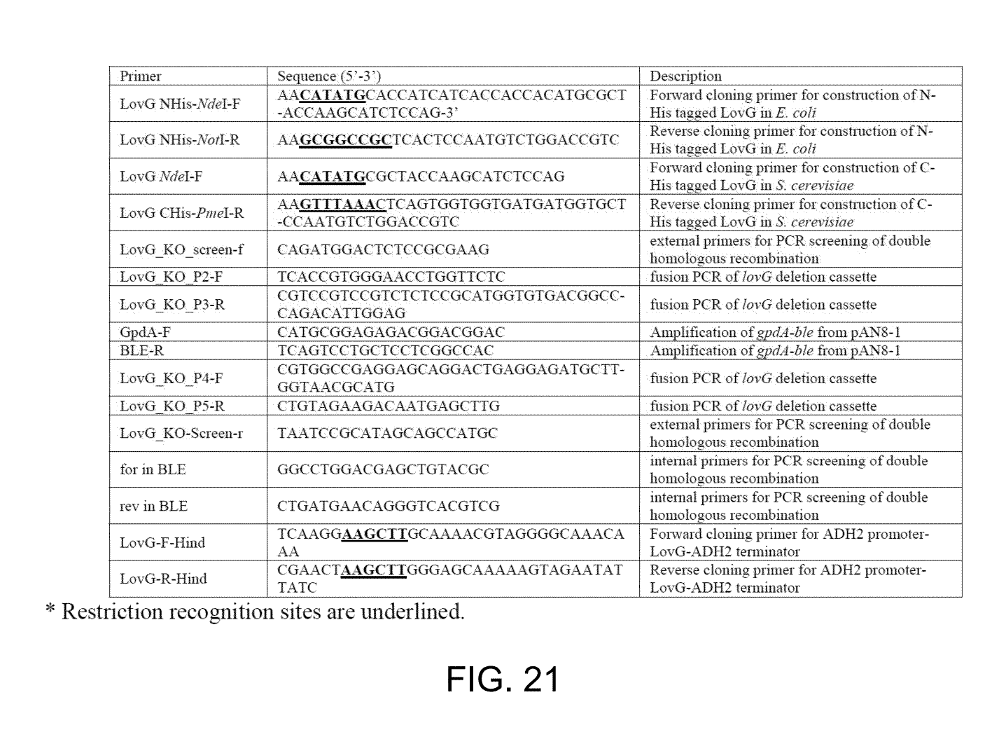

DNA sequences of lovG were duplicated from the genomic DNA of Aspergillus terreus NIH2624, using the primers LovG NHis-NdeI-F and LovG NHis-NotI-R listed in FIG. 21. The pCR product was ligated into pCR-Blunt vectors and subsequently digested with NdeI and Nod and then inserted into pET-23a (+) to yield pXW157.

### Cloning of C-his6 (SEQ ID NO: 15) LovG Cotransform Vector in Saccharomyces cerevisiae

The primer pair LovG NdeI-F and LovG CHis-PmeI-R was used to amplify lovG sequence. The resulting PCR product was ligated into pCR-Blunt vector and subsequently digested with NdeI and PmeI and then inserted into the digested pXW02 (an expression vector with a LEU2 marker) to create pXW161.

### Cloning of LovC-LovG Cotransform Vector in Saccharomyces cerevisiae

The primer pair LovG NdeIF and LovG CHis-PmeI-R was used to amplify ADH2p-lovG-ADH2t cassette. The resulting PCR product was ligated into pCR-Blunt vector and subsequently digested with HindIII and then inserted into the digested pXW06 (the LovC expression plasmid with a TRP1 marker) to create pSL05.

### II. Genetic Manipulation of Aspergillus terreus

### 1. Transformation of Disruption Cassettes into Aspergillus terreus

Inactivation cassette containing the bleomycin gene (ble) was constructed through fusion PCR[21], two 2 kb homologous regions in Aspergillus terreus NIH2624 genome were used to flank the resistant marker that is under the gpdA promoter[22]. Fusion PCR product was gel purified, ligated into pCR-Blunt vector and sequenced before using for transformation. Inactivation cassette was linearized by PCR and transformed into Aspergillus terreus NIH2624 through a polyethylene glycol-mediated protocol as described previously[23]. Transformants were grown on glucose minimal agar medium[24] supplemented with 1.2 M sorbitol and 250 μg/mL zeocin. To confirm correct integration of the inactivation cassette into the genome, gDNA was extracted from the transformants and used as template for PCR using primer pairs (FIG. 11).

### III. Protein Expression and Purification

### 1. Expression and Purification of LovG from E. coli

A chaperone plasmid pG-KJE8 (Takara) was co-transformed with pXW157 into E. coli BL21(DE3) strain through electroporation. The transformant was incubated at 37° C. in 1 L LB medium containing 35 μg/mL ampicillin, 25 μg/mL chloramphenicol, 0.5 mg/mL L-arabinose and 5 ng/mL tetracycline till an OD600 of 0.4-0.6. The cells were incubated on ice for 10 minutes, and then induced with isopropylthio-β-Dgalactoside (IPTG) (0.1 mM final concentration). The induced culture was incubated at 16° C. for 16 hours before the cells were harvested by centrifugation (2500 g, 15 minutes, 4° C.). The cells were re-suspended in 30 mL lysis buffer (50 mM Tris-HCl, 2 mM EDTA, 2 mM DTT, 500 mM NaCl, 5 mM imidazole, pH=7.9) and lysed through sonication on ice. Cellular debris was removed by centrifugation (30,000 g, 30 min, 4° C.). Ni-NTA agarose resin was added to the supernatant and the mixture was binding at 4° C. for at least 2 hours. LovG is elute together with chaperone protein GroEL with increasing concentration of imidazole in buffer A (50 mM Tris-HCl, 500 mM NaCl, pH=7.9) on a gravity flow column. The eluate was concentrated and exchanged into buffer E (50 mM Tris-HCl, 100 mM NaCl, pH=7.9). The resulting solution was loaded onto a sephacryl-S 100 size-exclusion column (GE Healthcare Life Sciences) under isocratic condition (0.5 mL/min flow rate with buffer E) for isolation of LovG from GroEL.

### 2. Expression and Purification of Proteins in S. cerevisiae

The expression plasmids were transformed into S. cerevisiae strain BJ5464-NpgA for expression of PKS proteins, by using S. c. EasyComp™ Transformation Kit (Invitrogen). For 1 L of yeast culture, the cells were grown at 28° C. in YPD media with 1% dextrose for 72 hours. The cells were harvested by centrifugation (2500 g, 20 minutes, 4° C.), resuspended in 20 mL lysis buffer (50 mM NaH2PO4 pH 8.0, 0.15 M NaCl, 10 mM imidazole) and lysed through sonication on ice. Cellular debris was removed by centrifugation (35,000 g, 1 hour, 4° C.). Ni-NTA agarose resin was added to the supernatant and the solution was stirred at 4° C. overnight. The protein/resin mixture was loaded into a gravity flow column and proteins were purified with increasing concentration of imidazole in Buffer A (50 mM Tris-HCl, pH=7.9, 2 mM EDTA, 2 mM DTT).

### 3. Storage of Purified Proteins

Purified proteins were concentrated and exchanged into buffer E (50 mM Tris-HCl, 100 mM NaCl, pH=7.9) +10% glycerol. The concentrated enzyme solutions were aliquoted and flash frozen. Protein concentrations were determined with the Bradford (Biorad) assay using BSA as a standard.

### IV. Characterization of LovG

### 1. In Vitro Turnover Assays

For in vitro assays in this study, a typical reaction volume is 100 μL and the final concentrations of enzymes were 10 μM. Cofactor concentrations are 2 mM NADPH, 1 mM SAM. The assays were carried out in 100 mM NaH2PO4 pH 7.4 buffer, at room temperature. MatB[25] (20 μM) was used to replenish malonyl-CoA from 10 mM CoA and 100 mM malonate or [2-13C]-malonate in presence of 20 mM ATP and 7 mM MgCl2. The reaction mixtures were incubated at room temperature for 12 hrs, then quenched and extracted twice with equal volume of 99% ethyl acetate (EA)/1% acetic acid (AcOH). The resultant organic extracts were evaporated to dryness, redissolved in 0.05M NaOH in methanol, and then analyzed on LC-MS. LC-MS was conducted with a Shimadzu 2010 EV Liquid Chromatography Mass Spectrometer by using both positive and negative electrospray ionization, and a Phenomenex Luna 5μ 2.0×100 mm C18 reverse-phase column. Samples were separated on a linear gradient of 5 to 95% CH3CN (v/v) in H2O supplemented with 0.05% (v/v) formic acid at a flow rate of 0.1 mL/min.

### 2. In Vivo Assays

For in vivo assays in this study, S. cerevisiae strain BJ5464-NpgA harboring the corresponding expression plasmids was inoculated to Yeast Synthetic Drop-Out medium without the nutruant like uracil, tryptophan or leucin. The cells were grown for 72 hours with constant shaking at 28° C. The seed culture was inoculated to 50 mLYPD (1% dextrose) to an initial OD₆₀₀ 0.1. The culture is incubated with constant shaking at 28° C. For induction of LovA and CPR, total 1% galactose was added to the culture after 24 hours of shaking. The sample culture was extracted twice with equal volume of 99% ethyl acetate (EA)/1% acetic acid (AcOH). The resultant organic extracts were evaporated to dryness, redissolved in 0.05M NaOH in methanol, and then analyzed on LC-MS. LC-MS analysis was conducted with a Shimadzu 2010 EV Liquid Chromatography Mass Spectrometer by using both positive and negative electrospray ionization, and a Phenomenex Luna 5α2.0×100 mm C18 reverse-phase column. Samples were separated on a linear gradient of 5 to 95% CH₃CN (v/v) in H₂O supplemented with 0.05% (v/v) formic acid at a flow rate of 0.1 mL/min.

### CONCLUSION

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

All publications, patents, and patent applications cited herein are hereby incorporated by reference in their entirety for all purposes.

### REFERENCES

[1] A. W. Alberts, J. Chen, G. Kuron, V. Hunt, J. Huff, C. Hoffman, J. Rothrock, M. Lopez, H. Joshua, E. Harris, A. Patchett, R. Monaghan, S. Currie, E. Stapley, G. Albers-Schonberg, O. Hensens, J. Hirshfield, K. Hoogsteen, J. Liesch, J. Springer, Proc. Natl. Acad. Sci. U.S.A. 1980, 77, 3957-3961.
[2] E. S. Istvan, J. Deisenhofer, Science 2001, 292, 1160-1164.
[3] X. K. Xie, Y. Tang, Appl. Environ. Microbiol. 2007, 73, 2054-2060.
[4] J. Kennedy, K. Auclair, S. G. Kendrew, C. Park, J. C. Vederas, C. R. Hutchinson, Science 1999, 284, 1368-1372.
[5] L. Hendrickson, C. R. Davis, C. Roach, D. K. Nguyen, T. Aldrich, P. C. McAda, C. D. Reeves, Chem. Biol. 1999, 6, 429-439.
[6] a) X. Xie, M. J. Meehan, W. Xu, P. C. Dorrestein, Y. Tang, J. Am. Chem. Soc. 2009, 131, 8388-8389; b) S. M. Ma, J. W. Li, J. W. Choi, H. Zhou, K. K. Lee, V. A. Moorthie, X. Xie, J. T. Kealey, N. A. Da Silva, J. C. Vederas, Y. Tang, Science 2009, 326, 589-592; c) J. Barriuso, D. T. Nguyen, J. W. Li, J. N. Roberts, G. MacNevin, J. L. Chaytor, S. L. Marcus, J. C. Vederas, D. K. Ro, J. Am. Chem. Soc. 2011, 133, 8078-8081.
[7] X. Xie, K. Watanabe, W. A. Wojcicki, C. C. Wang, Y. Tang, Chem. Biol. 2006, 13, 1161-1169.
[8] K. K. Lee, N. A. Da Silva, J. T. Kealey, Anal. Biochem. 2009, 394, 75-80.
[9] M. Wang, H. Zhou, M. Wirz, Y. Tang, C. N. Boddy, Biochemistry 2009, 48, 6288-6290.
[10] H. Zhou, K. Qiao, Z. Gao, M. J. Meehan, J. W. Li, X. Zhao, P. C. Dorrestein, J. C. Vederas, Y. Tang, J. Am. Chem. Soc. 2010, 132, 4530-4531.
[11] K. Auclair, J. Kennedy, C. R. Hutchinson, J. C. Vederas, Bioorg. Med. Chem. Lett. 2001, 11, 1527-1531.
[12] Y. Abe, T. Suzuki, C. Ono, K. Iwamoto, M. Hosobuchi, H. Yoshikawa, Mol. Genet. Genomics 2002, 267, 636-646.
[13] Y. P. Chen, G. F. Yuan, S. Y. Hsieh, Y. S. Lin, W. Y. Wang, L. L. Liaw, C. P. Tseng, J. Agric. Food Chem. 2010, 58, 287-293.
[14] D. Boettger, H. Bergmann, B. Kuehn, E. Shelest, C. Hertweck, ChemBioChem 2012, 13, 2363-2373.
[15] K. Nishihara, M. Kanemori, M. Kitagawa, H. Yanagi, T. Yura, Appl. Environ. Microbiol. 1998, 64, 1694-1699.
[16] T. Maier, M. Leibundgut, N. Ban, Science 2008, 321, 1315-1322.
[17] I. Fujii, A. Watanabe, U. Sankawa, Y. Ebizuka, Chem. Biol. 2001, 8, 189-197.
[18] A. L. Vagstad, S. B. Bumpus, K. Belecki, N. L. Kelleher, C. A. Townsend, J. Am. Chem. Soc. 2012, 134, 6865-6877.
[19] A. G. Newman, A. L. Vagstad, K. Belecki, J. R. Scheerer, C. A. Townsend, Chem. Commun. (Camb.) 2012, 48, 11772-11774.
[20] E. W. Jones, Methods Enzymol. 1991, 194, 428-453.
[21] E. Szewczyk, T. Nayak, C. E. Oakley, H. Edgerton, Y. Xiong, N. Taheri-Talesh, S. A. Osmani, B. R. Oakley, Nat. Protoc. 2006, 1, 3111-3120.
[22] P. J. Punt, I. E. Mattern, C. A. M. J. J. van den Hondel, Fungal Genet. Newsl. 1988, 35, 25-30.
[23] Y. H. Chooi, R. Cacho, Y. Tang, Chem. Biol. 2010, 17, 483-494.
[24] D. J. Cove, Biochim. Biophys. Acta 1966, 113, 51-56.
[25] J. H. An, Y. S. Kim, Eur J Biochem 1998, 257, 395-402.

TABLE 1
Aspergillus terreus POLYPEPTIDE AND POLYNUCLEOTIDE SEQUENCES.

Aspergillus terreus LovA: Dihydromonacolin L hydroxylase. Accession
Q9Y7C8
Kennedy et al. Science 284 (5418), 1368-1372 (1999)
MTVDALTQPHHLLSLAWNDTQQHGSWFAPLVTTSAGLLCLLLYLCSSGRRSDLPVFNPKTWWELTTMRAKRDFD
ANAPSWIESWFSQNDKPIRFIVDSGYCTILPSSMADEFRKMKELCMYKFLGTDFHSHLPGFDGFKEVTRDAHLI
TKVVMNQFQTQAPKYVKPLANEASGIITDIFGDSNEWHTVPVYNQCLDLVTRTVTFIMVGSKLAHNEEWLDIAK
HHAVTMAIQARQLRLWPVILRPLVHWLEPQGAKLRAQVRRARQLLDPIIQERRAERDACRAKGIEPPRYVDSIQ
WFEDTAKGKWYDAAGAQLAMDFAGIYGTSDLLIGGLVDIVRHPHLLEPLRDEIRTVIGQGGWTPASLYKLKLLD
SCLKESQRVKPVECATMRSYALQDVTFSNGTFIPKGELVAVAADRMSNPEVWPEPAKYDPYRYMRLREDPAKAF
SAQLENTNGDHIGFGWHPRACPGRFFASKEIKMMLAYLLIRYDWKVVPDEPLQYYRHSFSVRIHPTTKLMMRRR
DEDIRLPGSL
(SEQ ID NO: 1)
Aspergillus terreus LovB: Lovastatin nonaketide synthase. Accession Q9Y8A5
Kennedy et al. Science 284 (5418), 1368-1372 (1999)
MAQSMYPNEPIVVVGSGCRFPGDANTPSKLWELLQHPRDVQSRIPKERFDVDTFYHPDGKHHGRTNAPYAYVLQ
DDLGAFDAAFFNIQAGEAESMDPQHRLLLETVYEAVTNAGMRIQDLQGTSTAVYVGVMTHDYETVSTRDLESIP
TYSATGVAVSVASNRISYFFDWHGPSMTIDTACSSSLVAVHLAVQQLRTGQSSMAIAAGANLILGPMTFVLESK
LSMLSPSGRSRMWDAGADGYARGEAVCSVVLKTLSQALRDGDTIECVIRETGVNQDGRTTGITMPNHSAQEALI
KATYAQAGLDITKAEDRCQFFEAHGTGTPAGDPQEAEAIATAFFGHEQVARSDGNERAPLFVGSAKTVVGHTEG
TAGLAGLMKASFAVRHGVIPPNLLFDKISPRVAPFYKNLRIPTEATQWPALPPGQPRRASVNSFGFGGTNAHAI
IEEYMEPEQNQLRVSNNEDCPPMTGVLSLPLVLSAKSQRSLKIMMEEMLQFLQSHPEIHLHDLTWSLLRKRSVL
PFRRAIVGHSHETIRRALEDAIEDGIVSSDFTTEVRGQPSVLGIFTGQGAQWPGMLKNLIEASPYVRNIVRELD
DSLQSLPEKYRPSWTLLDQFMLEGEASNVQYATFSQPLCCAVQIVLVRLLEAARIRFTAVVGHSSGEIACAFAA
GLISASLAIRIAYLRGVVSAGGARGTPGAMLAAGMSFEEAQEICELDAFEGRICVAASNSPDSVTFSGDANAID
HLKGMLEDESTFARLLKVDTAYHSHHMLPCADPYMQALEECGCAVADAGSPAGSVPWYSSVDAENRQMAARDVT
AKYWKDNLVSPVLFSHAVQRAVVTHKALDIGIEVGCHPALKSPCVATIKDVLSGVDLAYTGCLERGKNDLDSFS
RALAYLWERFGASSFDADEFMRAVAPDRPCMSVSKLLPAYPWDRSRRYWVESRATRHHLRGPKPHLLLGKLSEY
STPLSFQWLNFVRPRDIEWLDGHALQGQTVFPAAGYIVMAMEAALMIAGTHAKQVKLLEILDMSIDKAVIFDDE
DSLVELNLTADVSRNAGEAGSMTISFKIDSCLSKEGNLSLSAKGQLALTIEDVNPRTTSASDQHHLPPPEEEHP
HMNRVNINAFYHELGLMGYNYSKDFRRLHNMQRADLRASGTLDFIPLMDEGNGCPLLLHPASLDVAFQTVIGAY
SSPGDRRLRCLYVPTHVDRITLVPSLCLATAESGCEKVAFNTINTYDKGDYLSGDIVVFDAEQTTLFQVENITF
KPFSPPDASTDHAMFARWSWGPLTPDSLLDNPEYWATAQDKEAIPIIERIVYFYIRSFLSQLTLEERQQAAFHL
QKQIEWLEQVLASAKEGRHLWYDPGWENDTEAQIEHLCTANSYHPHVRLVQRVGQHLLPTVRSNGNPFDLLDHD
GLLTEFYTNTLSFGPALHYARELVAQIAHRYQSMDILEIGAGTGGATKYVLATPQLGFNSYTYTDISTGFFEQA
REQFAPFEDRMVFEPLDIRRSPAEQGFEPHAYDLIIASNVLHATPDLEKTMAHARSLLKPGGQMVILEITHKEH
TRLGFIFGLFADWWAGVDDGRCTEPFVSFDRWDAILKRVGFSGVDSRTTDRDANLFPTSVFSTHAIDATVEYLD
APLASSGTVKDSYPPLVVVGGQTPQSQRLLNDIKAIMPPRPLQTYKRLVDLLDAEELPMKSTFVMLTELDEELF
AGLTEETFEATKLLLTYASNTVWLTENAWVQHPHQASTIGMLRSIRREHPDLGVHVLDVDAVETFDATFLVEQV
LRLEEHTDELASSTTWTQEPEVSWCKGRPWIPRLKRDLARNNRMNSSRRPIYEMIDSSRAPVALQTARDSSSYF
LESAETWFVPESVQQMETKTIYVHFSCPHALRVGQLGFFYLVQGHVQEGNREVPVVALAERNASIVHVRPDYIY
TEADNNLSEGGGSLMVTVLAAAVLAETVISTAKCLGVTDSILVLNPPSICGQMLLHAGEEIGLQVHLATTSGNR
SSVSAGDAKSWLTLHARDTDWHLRRVLPRGVQALVDLSADQSCEGLTQRMMKVLMPGCAHYRAADLFTDTVSTE
LHSGSRHQASLPAAYWEHVVSLARQGLPSVSEGWEVMPCTQFAAHADKTRPDLSTVISWPRESDEATLPTRVRS
IDAETLFAADKTYLLVGLTGDLGRSLGRWMVQHGACHIVLTSRNPQVNPKWLAHVEELGGRVTVLSMDVTSQNS
VEAGLAKLKDLHLPPVGGIAFGPLVLQDVMLNNMELPMMEMVLNPKVEGVRILHEKFSDPTSSNPLDFFVMFSS
IVAVMGNPGQANYSAANCYLQALAQQRVASGLAASTIDIGAVYGVGFVTRAELEEDFNAIRFMFDSVEEHELHT
LFAEAVVAGRRAVHQQEQQRKFATVLDMADLELTTGIPPLDPALKDRITFFDDPRIGNLKIPEYRGAKAGEGAA
GSKGSVKEQLLQATNLDQVRQIVIDGLSAKLQVTLQIPDGESVHPTIPLIDQGVDSLGAVTVGTWFSKQLYLDL
PLLKVLGGASITDLANEAAARLPPSSIPLVAATDGGAESTDNTSENEVSGREDTDLSAAATITEPSSADEDDTE
PGDEDVPRSHHPLSLGQEYSWRIQQGAEDPTVFNNTIGMFMKGSIDLKRLYKALRAVLRRHEIFRTGFANVDEN
GMAQLVFGQTKNKVQTIQVSDRAGAEEGYRQLVQTRYNPAAGDTLRLVDFFWGQDDHLLVVAYHRLVGDGSTTE
NIFVEAGQLYDGTSLSPHVPQFADLAARQRAMLEDGRMEEDLAYWKKMHYRPSSIPVLPLMRPLVGNSSRSDTP
NFQHCGPWQQHEAVARLDPMVAFRIKERSRKHKATPMQFYLAAYQVLLARLTDSTDLTVGLADTNRATVDEMAA
MGFFANLLPLRFRDFRPHITFGEHLIATRDLVREALQHARVPYGVLLDQLGLEVPVPTSNQPAPLFQAVFDYKQ
GQAESGTIGGAKITEVIATRERTPYDVVLEMSDDPTKDPLLTAKLQSSRYEAHHPQAFLESYMSLLSMFSMNPA
LKLA
(SEQ ID NO: 2)
Aspergillus terreus LovC: Enoyl reductase. Accession Q9Y7D0
Kennedy et al. Science 284 (5418), 1368-1372 (1999)
MGDQPFIPPPQQTALTVNDHDEVTVWNAAPCPMLPRDQVYVRVEAVAINPSDTKMRGQFATPWAFLGTDYAGTV
VAVGSDVTHIQVGDRVYGAQNEMCPRTPDQGAFSQYTVTRGRVWAKIPKGLSFEQAAALPAGISTAGLAMKLLG
LPLPSPSADQPPTHSKPVYVLVYGGSTATATVTMQMLRLSGYIPIATCSPHNFDLAKSRGAEEVFDYRAPNLAQ
TIRTYTKNNLRYALDCITNVESTTFCFAAIGRAGGHYVSLNPFPEHAATRKMVTTDWTLGPTIFGEGSTWPAPY
GRPGSEEERQFGEDLWRIAGQLVEDGRLVHHPLRVVQGGFDHIKQGMELVRKGELSGEKLVVRLEGP
(SEQ ID NO: 3)
Aspergillus terreus LovD transesterase. Accession Q9Y7D1
Kennedy et al. Science 284 (5418), 1368-1372 (1999)
MGSIIDAAAAADPVVLMETAFRKAVKSRQIPGAVIMARDCSGNLNYTRCFGARTVRRDECNQLPPLQVDTPCRL
ASATKLLTTIMALQCMERGLVDLDETVDRLLPDLSAMPVLEGFDDAGNARLRERRGKITLRHLLTHTSGLSYVF
LHPLLREYMAQGHLQSAEKFGIQSRLAPPAVNDPGAEWIYGANLDWAGKLVERATGLDLEQYLQENICAPLGIT
DMTFKLQQRPDMLARRADQTHRNSADGRLRYDDSVYFRADGEECFGGQGVFSGPGSYMKVLHSLLKRDGLLLQP
QTVDLMFQPALEPRLEEQMNQHMDASPHINYGGPMPMVLRRSFGLGGIIALEDLDGENWRRKGSLTFGGGPNIV
WQIDPKAGLCTLAFFQLEPWNDPVCRDLTRTFEHAIYAQYQQG
(SEQ ID NO: 4)
Aspergillus terreus LovF: Lovastatin diketide synthase. Accession Q9Y7D5
Kennedy et al. Science 284 (5418), 1368-1372 (1999)
MTPLDAPGAPAPIAMVGMGCRFGGGATDPQKLWKLLEEGGSAWSKIPPSRFNVGGVYHPNGQRVGSMHVRGGHF
LDEDPALFDASFFNMSTEVASCMDPQYRLILEVVYEALEAAGIPLEQVSGSKTGVFAGTMYHDYQGSFQRQPEA
LPRYFITGNAGTMLANRVSHFYDLRGPSVSIDTACSTTLTALHLAIQSLRAGESDMAIVAGANLLLNPDVFTTM
SNLGFLSSDGISYSFDSRADGYGRGEGVAAIVLKTLPDAVRDGDPIRLIVRETAINQDGRTPAISTPSGEAQEC
LIQDCYQKAQLDPKQTSYVEAHGTGTRAGDPLELAVISAAFPGQQIQVGSVKANIGHTEAVSGLASLIKVALAV
EKGVIPPNARFLQPSKKLLKDTHIQIPLCSQSWIPTDGVRRASINNFGFGGANAHAIVEQYGPFAETSICPPNG
YSGNYDGNLGTDQAHIYVLSAKDENSCMRMVSRLCDYATHARPADDLQLLANIAYTLGSRRSNFRWKAVCTAHS
LTGLAQNLAGEGMRPSKSADQVRLGWVFTGQGAQWFAMGRELIEMYPVFKEALLECDGYIKEMGSTWSIIEELS
RPETESRVDQAEFSLPLSTALQIALVRLLWSWNIQPVAVTSHSSGEAAAAYAIGALTARSAIGISYIRGALTAR
DRLASVHKGGMLAVGLSRSEVGIYIRQVPLQSEECLVVGCVNSPSSVTVSGDLSAIAKLEELLHADRIFARRLK
VTQAFHSSHMNSMTDAFRAGLTELFGADPSDAANASKDVIYASPRTGARLHDMNRLRDPIHWVECMLHPVEFES
AFRRMCLDENDHMPKVDRVIEIGPHGALGGPIKQIMQLPELATCDIPYLSCLSRGKSSLSTLRLLASELIRAGF
PVDLNAINFPRGCEAARVQVLSDLPPYPWNHETRYWKEPRISQSARQRKGPVHDLIGLQEPLNLPLARSWHNVL
RVSDLPWLRDHVVGSHIVFPGAGFVCMAVMGISTLCSSDHESDDISYILRDVNFAQALILPADGEEGIDLRLTI
CAPDQSLGSQDWQRFLVHSITADKNDWTEHCTGLVRAEMDQPPSSLSNQQRIDPRPWSRKTAPQELWDSLHRVG
IRHGPFFRNITCIESDGRGSWCTFAIADTASAMPHAYESQHIVHPTTLDSAVQAAYTTLPFAGSRIKSAMVPAR
VGCMKISSRLADLEARDMLRAQAKMHSQSPSALVTDVAVFDEADPVGGPVMELEGLVFQSLGASLGTSDRDSTD
PGNTCSSWHWAPDISLVNPGWLEKTLGTGIQEHEISLILELRRCSVHFIQEAMESLSVGDVERLSGHLAKFYAW
MQKQLACAQNGELGPESSSWTRDSEQARCSLRSRVVAGSTNGEMICRLGSVLPAILRREVDPLEVMMDGHLLSR
YYVDALKWSRSNAQASELVRLCCHKNPRARILEIGGGTGGCTQLVVDSLGPNPPVGRYDFTDVSAGFFEAARKR
FAGWQNVMDFRKLDIEDDPEAQGFVCGSYDVVLACQVLHATSNMQRTLTNVRKLLKPGGKLILVETTRDELDLF
FTFGLLPGWWLSEEPERQSTPSLSPTMWRSMLHTTGFNGVEVEARDCDSHEFYMISTMMSTAVQATPMSCSVKL
PEVLLVYVDSSTPMSWISDLQGEIRGRNCSVTSLQALRQVPPTEGQICVFLGEVEHSMLGSVTNDDFTLLTSML
QLAGGTLWVTQGATMKSDDPLKALHLGLLRTMRNESHGKRFVSLDLDPSRNPWTGDSRDAIVSVLDLISMSDEK
EFDYAERDGVIHVPRAFSDSINGGEEDGYALEPFQDSQHLLRLDIQTPGLLDSLHFTKRNVDTYEPDKLPDDWV
EIEPRAFGLNFRDIMVAMGQLESNVMGFECAGVVTSLSETARTIAPGLAVGDRVCALMNGHWASRVTTSRTNVV
RIPETLSFPHAASIPLAFTTAYISLYTVARILPGETVLIHAGAGGVGQAAIILAQLTGAEVFTTAGSETKRNLL
IDKFHLDPDHVFSSRDSSFVDGIKTRTRGKGVDVVLNSLAGPLLQKSFDCLARFGRFVEIGKKDLEQNSRLDMS
TFVRNVSFSSVDILYWQQAKPAEIFQAMSEVILLWERTAIGLIHPISEYPMSALEKAFRTMQSGQHVGKIVVTV
APDDAVLVRQERMPLFLKPNVSYLVAGGLGGIGRRICEWLVDRGARYLIILSRTARVDPVVTSLQERGCTVSVQ
ACDVADESQLEAALQQCRAEEMPPIRGVIQGAMVLKDALVSQMTADGFHAALRPKVQGSWNLHRIASDVDFFVM
LSSLVGVMGGAGQANYAAAGAFQDALAEHRMAHNQPAVTIDLGMVQSIGYVAETDSAVAERLQRIGYQPLHEEE
VLDVLEQAISPVCSPAAPTRPAVIVTGINTRPGPHWAHADWMQEARFAGIKYRDPLRDNHGALSLTPAEDDNLH
ARLNRAISQQESIAVIMEAMSCKLISMFGLTDSEMSATQTLAGIGVDSLVAIELRNWITAKFNVDISVFELMEG
RTIAKVAEVVLQRYKA
(SEQ ID NO: 5)
Aspergillus terreus LovG: Esterase. Accession Q0C8M2
Xu et al. Angew. Chem. Int. Ed. Engl. 52 (25), 6472-6475 (2013)
MRYQASPALAKAPRALLCIHGAGCSPAIFRVQLSKLRAALREDFEFVYVTAPFPSSAGPGILPVFADLGPYYSW
FESSSGNNNNGPSVGERLAAVHDPIRRTIVDWQTQHPHIPIVGAIGFSEGALVTTLLLWQQQMGRLPWLPRMSV
AMLICPWYQDEASQYMRNEVMENHDDDHDSKDTEWQEELVIRIPTLHLQGRDDFALAGSKMLVARHFSPREAQV
LEFAGQHQFPNRPRDVLEVINRFRKLCVTVQTLE
(SEQ ID NO: 6)
Aspergillus terreus NADPH--cytochrome P450 reductase. Accession Q0CMM0
MAQLDTLDLVVLVVLLVGSAAYFTKGTYWAVPKDPYAASGPAMNGGAKAGKSRDIIEKMEETGKNCVIFYGSQT
GTAEDYASRLAKEGSQRFGLKTMVADLEDYDYENLDKFPEDKVAFFVMATYGEGEPTDNAVEFYQFISGEDVAF
ESGASADDKPLSSLKYVTFGLGNNTYEHYQAMVRNLDAALTKLGAQRIGDAGEGDDGAGTMEEDFLAWKEPMWT
ALSEAMNLQEREAVYEPVFSVTEDESLSPEDEAVYLGEPTKGHRDGTPSGPYSAHNPFIAPIVESRELFNVKDR
NCLHMEISIAGSNLSYQTGDHIAIWPTNAGAEVDRFLQVFGLENKRHSVINIKGIDVTAKVPIPTPTTYDAAVR
YYMEIAAPVSRQFVATLAAFAPDEETKAEIVRLGSDKDYFHEKISNQCFTIAQALQSVTSKPFSAVPFSLLIEG
LNKLQPRYYSISSSSMVQKDKISITAVVESTRLPGAAHLVKGVTTNYLLALKQKQNGDPSPDPHGLTYTITGPR
NKYDGIHVPVHVRHSNFKLPSDPSRPIIMVGPGTGVAPFRGFIQERAALAAKGENVGPTVLFFGCRRRDEDFMY
ADEFKTYQEQLGDKLQIITAFSRETSQKVYVQHRLREHSDLVSSLLKQKANFYVCGDAANMAREVNLVLGQIIA
QQRGLPAERAEEMVKHMRSSGSYQEDVWS
(SEQ ID NO: 7)
Aspergillus terreus LovA: Dihydromonacolin L hydroxylase.
ATGACTGTCGACGCGCTCACACAGCCGCACCACCTTCTGTCGCTGGCTTGGAATGACACGCAGCAACATGGCTC
GTGGTTTGCGCCCTTGGTCACTACCAGTGCGGGGCTACTATGCCTTCTTCTTTACCTGTGCTCGAGTGGCCGGA
GATCTGATCTGCCGGTGTTCAATCCGAAAACATGGTGGGAACTGACGACCATGAGGGCCAAACGGGATTTTGAT
GCGAATGCACCGTCATGGATTGAGAGCTGGTTCTCGCAAAATGATAAGCCCATTCGGTTCATAGTCGACTCTGG
GTACTGCACCATTCTTCCCTCCTCTATGGCCGATGAGTTTCGCAAGATGAAAGAGCTCTGTATGTACAAGTTCT
TGGGCACGGACTTTCACTCTCATCTTCCCGGATTCGATGGATTCAAGGAAGTCACGAGGGATGCACATCTCATC
ACCAAGGTGGTTATGAACCAGTTCCAGACCCAAGCTCCCAAGTACGTCAAGCCTCTTGCCAATGAAGCCAGCGG
GATTATCACGGATATTTTTGGCGACAGCAATGAATGGCACACAGTGCCTGTCTATAACCAGTGTCTGGACTTAG
TGACCCGAACAGTGACTTTTATTATGGTCGGGAGCAAGTTAGCCCATAATGAGGAGTGGCTTGACATCGCCAAG
CACCACGCGGTGACGATGGCAATTCAAGCGCGCCAGCTGCGCCTCTGGCCCGTCATTCTGCGCCCCCTTGTACA
TTGGCTCGAGCCCCAGGGAGCCAAACTCCGGGCGCAGGTTCGACGAGCCCGGCAACTTCTCGATCCCATTATCC
AGGAGCGACGTGCGGAAAGAGATGCCTGCCGGGCAAAGGGCATTGAGCCGCCTCGCTACGTAGACTCGATCCAG
TGGTTCGAGGATACTGCCAAGGGGAAATGGTACGATGCAGCCGGGGCGCAACTGGCCATGGACTTTGCTGGTAT
CTACGGAACCTCCGACCTGCTGATCGGTGGGTTGGTGGACATCGTCCGACATCCCCATCTCCTTGAGCCCCTCC
GTGATGAGATCCGGACGGTCATCGGCCAAGGGGGTTGGACACCTGCCTCGCTGTACAAGCTCAAACTGCTGGAT
AGTTGTCTCAAGGAGTCACAGCGCGTCAAGCCCGTCGAATGTGCCACCATGCGCAGCTATGCATTGCAGGATGT
GACTTTCTCCAATGGAACCTTTATCCCAAAAGGAGAGCTGGTGGCGGTAGCTGCCGACCGCATGAGCAACCCCG
AGGTCTGGCCAGAGCCGGCAAAATACGATCCTTACCGGTATATGCGCCTGCGAGAGGACCCGGCTAAAGCGTTC
AGTGCCCAACTGGAGAACACCAACGGGGACCACATCGGCTTCGGTTGGCATCCACGGGCTTGCCCCGGCCGGTT
CTTTGCCTCTAAGGAGATCAAGATGATGTTAGCCTACTTGCTCATACGATACGACTGGAAGGTGGTCCCCGACG
AACCGTTGCAGTACTACCGCCATTCTTTCAGCGTGCGCATTCATCCCACCACGAAGCTCATGATGCGCCGGCGC
GACGAGGATATCCGCCTTCCTGGTTCACTA
(SEQ ID NO: 8)
Aspergillus terreus LovB: Lovastatin nonaketide synthase. Accession Q9Y8A5
ATGGCTCAATCTATGTATCCTAATGAGCCTATTGTCGTGGTCGGCAGTGGTTGTCGCTTCCCTGGTGACGCCAA
CACACCCTCCAAGCTCTGGGAGCTACTCCAGCATCCTCGCGATGTGCAGAGTCGAATCCCCAAAGAACGATTTG
ACGTCGACACATTTTATCACCCGGACGGGAAGCACCACGGGCGAACAAATGCACCCTACGCCTATGTTCTCCAA
GACGATCTGGGCGCCTTCGATGCGGCCTTCTTCAATATCCAGGCTGGAGAGGCCGAGAGTATGGACCCCCAGCA
CCGGCTGTTGCTGGAGACGGTGTACGAGGCCGTAACGAATGCTGGAATGCGTATCCAGGATCTGCAGGGAACTT
CGACTGCTGTTTACGTCGGGGTGATGACGCACGACTATGAGACTGTCTCAACCCGCGACCTGGAGAGCATCCCC
ACCTACTCGGCGACGGGTGTCGCGGTCAGTGTTGCGTCCAACCGCATCTCGTATTTTTTTGACTGGCATGGACC
AAGTATGACGATCGATACGGCATGCAGCTCGTCGTTGGTTGCCGTTCATCTGGCGGTGCAACAGCTACGGACGG
GTCAAAGCTCCATGGCAATTGCTGCGGGTGCGAATCTGATTCTGGGGCCCATGACATTCGTCCTTGAAAGCAAA
TTGAGCATGCTATCCCCCTCGGGTCGATCCCGCATGTGGGACGCCGGAGCTGACGGCTATGCCAGAGGCGAAGC
TGTTTGCTCTGTAGTGTTGAAGACATTGAGTCAAGCCTTGCGCGATGGGGACACGATTGAATGTGTCATCCGAG
AAACTGGGGTGAATCAAGATGGCCGAACGACCGGAATTACGATGCCGAACCATAGTGCTCAGGAGGCACTCATC
AAGGCTACCTACGCCCAGGCTGGCCTTGACATCACCAAGGCCGAGGACAGGTGCCAATTCTTCGAGGCTCATGG
GACTGGTACTCCGGCCGGAGATCCCCAGGAGGCGGAGGCCATTGCAACAGCCTTCTTCGGCCACGAGCAGGTAG
CACGCAGCGACGGAAACGAGAGGGCCCCTCTGTTCGTGGGCAGTGCGAAAACTGTTGTCGGGCACACCGAGGGC
ACGGCCGGTCTGGCTGGTCTCATGAAGGCGTCGTTCGCTGTCCGCCATGGGGTAATCCCCCCCAACCTGCTGTT
CGACAAAATCAGCCCGCGAGTCGCCCCATTCTATAAAAACCTGAGGATTCCGACAGAAGCTACCCAATGGCCAG
CTCTCCCACCCGGACAACCGCGCCGCGCCAGTGTCAACTCCTTTGGATTCGGCGGCACGAATGCGCATGCCATT
ATTGAGGAATACATGGAGCCAGAGCAAAACCAGCTGCGAGTCTCGAATAATGAGGACTGCCCACCCATGACCGG
TGTCCTGAGTTTACCCTTAGTCCTCTCGGCGAAGTCCCAGCGCTCCTTAAAGATAATGATGGAGGAGATGCTGC
AATTCCTTCAGTCTCACCCCGAGATACACTTGCACGACCTCACCTGGTCCTTACTGCGCAAGCGGTCAGTTCTA
CCCTTCCGCCGGGCTATTGTCGGCCATAGTCATGAAACCATCCGCCGGGCTTTGGAGGATGCCATCGAGGATGG
TATTGTGTCGAGCGACTTCACTACGGAGGTCAGAGGCCAGCCATCGGTGTTGGGAATCTTCACCGGGCAGGGGG
CGCAGTGGCCGGGGATGTTAAAGAATCTGATAGAGGCATCGCCATATGTGCGGAACATAGTGAGGGAGCTGGAC
GACTCCCTGCAGAGCTTGCCGGAAAAATACCGGCCCTCGTGGACGCTACTGGACCAGTTCATGCTAGAAGGAGA
GGCCTCCAACGTCCAATATGCTACTTTCTCCCAGCCATTATGCTGCGCGGTGCAAATTGTCCTGGTCCGTCTCC
TTGAAGCCGCGAGAATACGATTCACGGCTGTTGTTGGACATAGCTCCGGCGAAATTGCTTGCGCCTTTGCTGCC
GGGCTCATCAGTGCCTCGTTGGCGATTCGGATTGCTTACTTACGTGGAGTCGTCTCGGCAGGGGGCGCCAGAGG
CACACCGGGAGCCATGTTGGCCGCCGGGATGTCCTTTGAGGAAGCACAAGAGATCTGCGAGTTGGATGCCTTTG
AGGGCCGCATCTGCGTGGCTGCCAGCAATTCCCCAGACAGTGTAACTTTCTCTGGCGACGCGAACGCAATTGAT
CACCTGAAGGGCATGTTGGAGGATGAGTCCACTTTTGCGAGACTGCTCAAGGTCGATACAGCGTACCACTCGCA
TCATATGCTTCCATGTGCAGACCCATATATGCAAGCCCTAGAAGAGTGTGGTTGTGCTGTTGCCGATGCAGGTT
CCCCAGCCGGAAGTGTACCCTGGTATTCGTCCGTGGACGCCGAGAACAGGCAAATGGCAGCAAGAGACGTGACC
GCCAAGTACTGGAAAGATAACTTAGTATCTCCGGTGCTATTCTCCCACGCAGTGCAGCGGGCAGTCGTCACGCA
CAAGGCGCTGGATATCGGGATTGAAGTGGGCTGTCACCCAGCTCTCAAGAGCCCATGCGTCGCCACCATCAAGG
ATGTCCTATCTGGGGTTGACCTGGCGTATACAGGTTGCTTGGAGCGAGGAAAGAATGATCTCGATTCATTCTCT
CGAGCACTGGCATATCTCTGGGAAAGGTTTGGTGCCTCCAGTTTCGATGCGGACGAGTTCATGCGTGCAGTCGC
GCCTGATCGGCCCTGTATGAGTGTGTCGAAGCTCCTACCGGCCTATCCATGGGACCGCTCTCGTCGCTACTGGG
TGGAATCCCGAGCAACTCGCCACCATCTTCGAGGGCCCAAGCCCCATCTTCTATTAGGAAAGCTCTCCGAATAC
AGCACTCCGCTAAGCTTCCAGTGGCTGAATTTTGTGCGCCCACGAGACATTGAATGGCTTGATGGACATGCATT
GCAAGGCCAGACTGTCTTCCCTGCGGCCGGCTATATCGTCATGGCAATGGAAGCAGCCTTAATGATTGCTGGCA
CCCACGCAAAGCAGGTCAAGTTACTGGAGATCTTGGATATGAGCATTGACAAGGCGGTGATATTTGACGACGAA
GACAGCTTGGTTGAGCTCAACCTGACAGCTGACGTGTCTCGCAACGCCGGCGAAGCAGGTTCAATGACCATAAG
CTTCAAGATCGATTCCTGTCTATCGAAGGAGGGTAACCTATCCCTATCAGCCAAGGGCCAACTGGCCCTAACGA
TAGAAGATGTCAATCCCAGGACGACTTCCGCTAGCGACCAGCACCATCTTCCCCCGCCAGAAGAGGAACATCCT
CATATGAACCGTGTCAACATCAATGCTTTCTACCACGAGCTGGGGTTGATGGGGTACAACTACAGTAAGGACTT
CCGGCGTCTCCATAACATGCAACGAGCAGATCTTCGAGCCAGCGGCACCTTAGACTTCATTCCTCTGATGGACG
AGGGTAATGGCTGTCCTCTCCTGCTGCATCCTGCATCATTGGACGTCGCCTTCCAGACTGTCATCGGCGCATAC
TCCTCCCCAGGTGATCGGCGTCTACGCTGTCTGTATGTACCCACTCACGTTGATCGCATCACACTTGTCCCATC
CCTTTGCCTGGCAACGGCTGAGTCCGGATGCGAGAAGGTTGCCTTCAATACTATCAATACGTACGACAAGGGAG
ACTACTTGAGCGGTGACATTGTGGTGTTTGACGCGGAGCAGACCACCCTGTTCCAGGTTGAAAATATTACTTTT
AAGCCCTTTTCACCCCCGGATGCTTCAACTGACCATGCGATGTTTGCCCGATGGAGCTGGGGTCCGTTGACTCC
GGACTCGCTGCTGGATAACCCGGAGTATTGGGCCACCGCGCAGGACAAGGAGGCGATTCCTATTATCGAACGCA
TCGTCTACTTCTATATCCGATCGTTCCTCAGTCAGCTTACGCTGGAGGAGCGCCAGCAGGCAGCCTTCCATTTG
CAGAAGCAGATCGAGTGGCTCGAACAAGTCCTGGCCAGCGCCAAGGAGGGTCGTCACCTATGGTACGACCCCGG
GTGGGAGAATGATACTGAGGCCCAGATTGAGCACCTTTGTACTGCTAACTCCTACCACCCTCATGTTCGCCTGG
TTCAGCGAGTCGGCCAACACCTGCTCCCCACCGTACGATCGAACGGCAACCCATTCGACCTTCTGGACCACGAT
GGGCTCCTGACGGAGTTCTATACCAACACACTCAGCTTCGGACCCGCACTACACTACGCCCGGGAATTGGTGGC
GCAGATCGCCCATCGCTATCAGTCAATGGATATTCTGGAGATTGGAGCAGGGACCGGCGGCGCTACCAAGTACG
TGTTGGCCACGCCCCAGCTGGGGTTCAACAGCTACACATACACCGATATCTCCACCGGATTCTTCGAGCAAGCG
CGGGAGCAATTTGCCCCCTTCGAGGACCGGATGGTGTTTGAACCCCTCGATATCCGCCGCAGTCCCGCCGAGCA
GGGCTTCGAGCCGCATGCCTATGATCTGATCATTGCCTCCAATGTGCTACATGCGACACCCGACCTAGAGAAAA
CCATGGCTCACGCCCGCTCTCTGCTCAAGCCTGGAGGCCAGATGGTTATTCTGGAGATTACCCACAAAGAACAC
ACACGGCTCGGGTTTATCTTTGGTCTGTTCGCCGACTGGTGGGCTGGGGTGGATGATGGTCGCTGCACTGAGCC
GTTTGTCTCGTTCGACCGCTGGGATGCGATCCTAAAGCGTGTCGGGTTTTCCGGTGTGGACAGTCGCACCACGG
ATCGGGACGCAAATCTATTCCCGACCTCTGTGTTTAGTACCCATGCAATTGACGCCACCGTGGAGTACTTAGAC
GCGCCGCTTGCCAGCAGCGGCACCGTCAAGGACTCTTACCCTCCCTTGGTGGTGGTAGGAGGGCAGACCCCCCA
ATCTCAGCGTCTCCTGAACGATATAAAAGCGATCATGCCTCCTCGTCCGCTCCAGACATACAAGCGCCTCGTGG
ATTTGCTAGACGCGGAGGAGCTGCCGATGAAGTCCACGTTTGTCATGCTCACGGAGCTGGACGAGGAATTATTC
GCCGGGCTCACTGAAGAGACCTTCGAGGCAACCAAGCTGCTGCTCACGTACGCCAGCAATACGGTCTGGCTGAC
AGAAAATGCCTGGGTCCAACATCCTCACCAGGCGAGCACGATCGGCATGCTACGCTCCATCCGCCGGGAGCATC
CTGACTTGGGAGTTCATGTTCTGGACGTCGACGCGGTTGAAACCTTCGATGCAACCTTCCTGGTTGAACAGGTG
CTTCGGCTTGAGGAGCATACGGATGAGCTGGCCAGTTCAACTACATGGACTCAAGAACCCGAGGTCTCCTGGTG
TAAAGGCCGCCCGTGGATTCCTCGTCTGAAGCGCGATCTGGCTCGCAATAACCGAATGAACTCCTCGCGCCGTC
CCATATACGAGATGATCGATTCGTCGCGGGCTCCCGTGGCATTACAGACGGCTCGGGATTCATCATCCTACTTC
TTGGAGTCCGCTGAAACCTGGTTTGTGCCTGAGAGTGTTCAGCAGATGGAAACAAAGACGATCTATGTCCACTT
TAGCTGTCCCCATGCGCTTAGGGTCGGACAGCTCGGGTTTTTCTATCTTGTGCAGGGTCACGTCCAGGAGGGCA
ATCGCGAAGTGCCCGTCGTGGCCTTAGCAGAGCGTAACGCATCCATTGTGCACGTTCGTCCCGATTATATATAT
ACTGAGGCAGATAACAATCTGTCTGAGGGTGGTGGCAGCCTTATGGTAACCGTCCTCGCCGCGGCGGTGTTGGC
GGAGACGGTGATCAGTACCGCCAAGTGCCTGGGGGTAACTGACTCAATCCTCGTTCTGAATCCCCCCAGCATAT
GTGGGCAGATGTTGCTCCATGCTGGTGAAGAGATCGGTCTTCAAGTTCATCTGGCCACCACTTCTGGCAACAGG
AGTTCGGTTTCTGCTGGAGACGCCAAGTCCTGGCTAACATTGCATGCTCGCGACACGGACTGGCACCTGCGACG
GGTACTGCCCCGGGGTGTCCAGGCTTTAGTCGACTTATCAGCCGACCAGAGCTGTGAAGGTTTGACTCAGAGGA
TGATGAAAGTTCTGATGCCTGGCTGTGCCCATTACCGTGCGGCAGACCTGTTCACAGACACCGTTTCCACTGAA
TTGCATAGCGGATCGCGGCATCAAGCTTCACTGCCCGCCGCATATTGGGAGCATGTGGTATCCTTAGCCCGCCA
GGGACTTCCTAGTGTCAGCGAGGGGTGGGAGGTGATGCCGTGCACTCAATTTGCAGCGCATGCCGACAAGACGC
GCCCGGATCTCTCGACAGTTATTTCCTGGCCCCGGGAGTCGGACGAGGCTACGCTTCCTACCAGGGTTCGCTCC
ATTGACGCTGAGACCCTCTTTGCGGCCGACAAAACATATCTCCTGGTCGGACTGACTGGAGATCTTGGACGATC
ACTAGGTCGTTGGATGGTCCAGCATGGGGCCTGCCACATTGTACTTACGAGCAGAAATCCGCAGGTGAACCCCA
AGTGGCTGGCGCATGTTGAAGAACTGGGTGGTCGAGTCACTGTTCTTTCCATGGACGTGACAAGCCAAAACTCA
GTGGAAGCTGGCCTGGCTAAACTCAAGGATCTGCATCTGCCACCAGTGGGGGGTATTGCCTTTGGCCCTCTGGT
TCTGCAGGATGTGATGCTAAATAATATGGAACTGCCAATGATGGAGATGGTGCTCAACCCCAAGGTCGAAGGCG
TCCGCATCCTGCACGAGAAGTTCTCCGATCCGACCAGTAGCAACCCTCTCGACTTCTTCGTGATGTTCTCCTCG
ATTGTGGCCGTCATGGGCAACCCGGGTCAGGCTAACTACAGTGCGGCTAACTGCTACCTTCAAGCGCTGGCGCA
GCAGCGAGTTGCATCCGGATTAGCAGCGTCCACCATCGACATCGGTGCCGTGTACGGCGTTGGGTTCGTCACTC
GGGCGGAGCTGGAGGAGGACTTTAATGCAATTCGGTTCATGTTCGATTCGGTTGAGGAACATGAACTGCATACA
CTGTTTGCTGAGGCAGTGGTGGCCGGTCGACGAGCCGTGCACCAGCAAGAGCAGCAGCGGAAGTTCGCGACAGT
GCTCGACATGGCTGATCTGGAACTGACAACCGGAATTCCGCCCCTGGATCCAGCCCTCAAAGATCGGATCACCT
TCTTCGACGACCCCCGCATAGGCAACTTAAAAATTCCGGAGTACCGAGGGGCCAAAGCAGGCGAAGGGGCAGCC
GGCTCCAAGGGCTCGGTCAAAGAACAGCTCTTGCAGGCGACGAACCTGGACCAGGTCCGTCAGATCGTCATCGA
TGGACTCTCCGCGAAGCTGCAGGTGACCCTGCAGATCCCCGATGGGGAAAGCGTGCATCCCACCATCCCACTAA
TCGATCAGGGGGTGGACTCTCTGGGCGCGGTCACCGTGGGAACCTGGTTCTCCAAGCAGCTGTACCTTGATTTG
CCACTCCTGAAAGTGCTTGGGGGTGCTTCGATCACCGATCTCGCTAATGAGGCTGCTGCGCGATTGCCACCTAG
CTCCATTCCCCTCGTCGCAGCCACCGACGGGGGTGCAGAGAGCACTGACAATACTTCCGAGAATGAAGTTTCGG
GACGCGAGGATACTGACCTTAGTGCCGCCGCCACCATCACTGAGCCCTCGTCTGCCGACGAAGACGATACGGAG
CCGGGCGACGAGGACGTCCCGCGTTCCCACCATCCACTGTCTCTCGGGCAAGAATACTCCTGGAGAATCCAGCA
GGGAGCCGAAGACCCCACCGTCTTTAACAACACCATTGGTATGTTCATGAAGGGCTCTATTGACCTTAAACGGC
TGTACAAGGCGTTGAGAGCGGTCTTGCGCCGCCACGAGATCTTCCGCACGGGGTTTGCCAACGTGGATGAGAAC
GGGATGGCCCAGCTGGTGTTTGGTCAAACCAAAAACAAAGTCCAGACCATCCAAGTGTCTGACCGAGCCGGCGC
CGAAGAGGGCTACCGACAACTGGTGCAGACACGGTATAACCCTGCCGCAGGAGACACCTTGCGGCTGGTGGACT
TCTTCTGGGGCCAGGACGACCATCTGCTGGTTGTGGCTTACCACCGACTCGTCGGGGATGGATCTACTACAGAG
AACATCTTCGTCGAAGCGGGCCAGCTCTACGACGGCACGTCGCTAAGTCCACATGTCCCTCAGTTTGCGGACCT
GGCGGCACGGCAACGCGCAATGCTCGAGGATGGGAGAATGGAGGAGGATCTCGCGTACTGGAAGAAAATGCATT
ACCGACCGTCCTCAATTCCAGTGCTCCCACTGATGCGGCCCCTGGTAGGTAACAGTAGCAGGTCCGATACTCCA
AATTTCCAGCACTGTGGACCCTGGCAGCAGCACGAAGCCGTGGCGCGACTTGATCCGATGGTGGCCTTCCGCAT
CAAGGAGCGCAGTCGCAAGCACAAGGCGACGCCGATGCAGTTCTATCTGGCGGCGTATCAGGTGCTGTTGGCGC
GCCTCACCGACAGCACCGATCTCACCGTGGGCCTCGCCGACACCAACCGTGCGACTGTCGACGAGATGGCGGCC
ATGGGGTTCTTCGCCAACCTCCTTCCCCTGCGCTTCCGGGATTTCCGCCCCCATATAACGTTTGGCGAGCACCT
TATCGCCACCCGTGACCTGGTGCGTGAGGCCTTGCAGCACGCCCGCGTGCCCTACGGCGTCCTCCTCGATCAAC
TGGGGCTGGAGGTCCCGGTCCCGACCAGCAATCAACCTGCGCCTTTGTTCCAGGCCGTCTTCGATTACAAGCAG
GGCCAGGCGGAAAGTGGAACGATTGGGGGTGCCAAGATAACCGAGGTGATTGCCACGCGCGAGCGCACCCCTTA
CGATGTCGTGCTGGAGATGTCGGATGATCCCACCAAGGATCCGCTGCTCACGGCCAAGTTACAGAGTTCCCGCT
ACGAGGCTCACCACCCTCAAGCCTTCTTGGAGAGCTACATGTCCCTTCTCTCTATGTTCTCGATGAATCCCGCC
CTGAAGCTGGCA
(SEQ ID NO: 9)
Aspergillus terreus LovC: Enoyl reductase. Accession Q9Y7D0
ATGGGCGACCAGCCATTCATTCCACCACCGCAGCAAACAGCGCTGACGGTAAATGACCATGATGAAGTCACCGT
CTGGAATGCCGCACCCTGCCCCATGCTGCCCCGCGACCAGGTATACGTCCGCGTCGAGGCCGTGGCGATCAATC
CCAGTGACACGAAGATGCGCGGACAGTTTGCCACGCCCTGGGCGTTTCTCGGAACGGACTATGCCGGCACGGTC
GTCGCAGTGGGTTCGGACGTGACTCATATCCAAGTGGGTGACCGGGTCTACGGGGCACAGAACGAGATGTGCCC
ACGCACCCCGGATCAGGGGGCATTCTCGCAGTACACGGTCACGCGAGGCCGTGTTTGGGCCAAGATCCCCAAGG
GCTTGTCGTTCGAGCAGGCTGCCGCGCTACCTGCGGGCATCAGTACCGCTGGATTGGCGATGAAGTTGCTTGGG
CTGCCTTTGCCATCGCCTTCGGCAGACCAGCCACCCACCCACTCCAAGCCGGTGTATGTGTTGGTCTATGGGGG
CAGTACGGCCACTGCCACTGTCACTATGCAAATGCTCCGCCTGTCCGGATATATTCCAATTGCAACATGCTCCC
CCCACAATTTCGACCTGGCCAAATCGCGCGGCGCAGAGGAGGTCTTTGACTATCGGGCCCCGAATCTCGCGCAG
ACGATCCGTACCTACACCAAGAACAATCTCCGCTATGCTCTCGACTGTATCACCAACGTCGAGTCCACCACATT
CTGCTTCGCAGCCATCGGCCGCGCGGGGGGGCACTACGTCTCCCTGAACCCGTTCCCTGAACACGCGGCCACGC
GCAAGATGGTCACGACCGACTGGACCCTGGGGCCGACCATCTTTGGCGAGGGATCAACCTGGCCCGCCCCCTAT
GGGCGTCCCGGCAGTGAGGAAGAGCGGCAGTTCGGCGAGGATCTGTGGCGCATCGCGGGGCAGCTCGTCGAAGA
TGGACGCCTCGTCCATCATCCGTTGCGCGTGGTGCAGGGCGGCTTCGATCACATTAAGCAAGGCATGGAGCTCG
TCCGGAAGGGAGAGCTGTCGGGGGAGAAACTCGTGGTTCGGCTCGAGGGGCCG
(SEQ ID NO: 10)
Aspergillus terreus LovD transesterase. Accession Q9Y7D1
ATGGGATCCATCATTGATGCTGCTGCGGCAGCGGATCCGGTTGTTCTGATGGAAACCGCCTTCCGCAAGGCCGT
GAAATCCAGGCAGATCCCCGGGGCGGTCATCATGGCCCGAGATTGCAGTGGCAATCTAAATTATACGCGCTGCT
TCGGGGCTCGGACGGTGCGACGGGACGAGTGCAATCAGCTGCCGCCGCTACAGGTCGACACCCCCTGCCGGCTC
GCCAGTGCGACCAAGCTGCTGACCACGATCATGGCCCTACAATGCATGGAGCGCGGTCTCGTGGACTTGGATGA
GACGGTGGATAGGCTGCTTCCGGATTTGAGCGCGATGCCCGTGCTGGAGGGGTTTGACGACGCGGGAAACGCAA
GATTGCGAGAGCGTCGGGGGAAGATCACGCTGCGGCACCTGCTGACGCATACATCGGGACTGTCGTACGTCTTC
CTCCATCCGTTGCTCCGGGAATACATGGCCCAGGGCCACCTCCAGTCGGCAGAAAAGTTTGGCATCCAGAGTCG
CCTGGCGCCGCCGGCCGTCAACGACCCTGGGGCGGAGTGGATCTACGGCGCCAACCTGGACTGGGCGGGTAAGC
TCGTCGAGCGGGCCACCGGCCTCGACCTGGAGCAGTACCTGCAGGAGAATATCTGTGCGCCGCTGGGCATCACC
GACATGACCTTTAAGCTGCAGCAACGGCCGGATATGCTTGCGCGCCGGGCCGACCAAACGCACCGCAACTCGGC
GGATGGGCGCCTGCGCTACGACGACTCGGTCTACTTCCGGGCCGATGGAGAGGAGTGCTTCGGCGGCCAGGGGG
TGTTCTCGGGCCCTGGGTCCTATATGAAGGTGCTTCACTCGCTGTTGAAGCGAGACGGGCTCCTGCTGCAGCCA
CAGACCGTGGACTTGATGTTTCAGCCTGCCCTCGAGCCGCGACTCGAAGAGCAGATGAACCAGCACATGGACGC
CAGCCCACATATCAACTACGGTGGGCCGATGCCCATGGTCCTTCGTCGCAGCTTTGGGCTGGGGGGGATCATCG
CCTTGGAGGATCTGGACGGAGAGAACTGGCGCCGAAAAGGTTCCTTGACCTTTGGGGGTGGCCCAAACATTGTG
TGGCAAATCGACCCCAAAGCCGGCCTGTGCACCCTTGCGTTCTTCCAACTGGAACCCTGGAATGACCCGGTCTG
TCGTGATCTGACACGCACATTCGAGCATGCCATCTATGCGCAGTACCAGCAGGGT
(SEQ ID NO: 11)
Aspergillus terreus LovF: Lovastatin diketide synthase. Accession Q9Y7D5
ATGACACCATTAGATGCGCCCGGTGCGCCTGCTCCCATAGCTATGGTTGGCATGGGCTGCAGATTTGGCGGAGG
CGCAACAGATCCCCAGAAACTGTGGAAATTGCTGGAGGAAGGAGGGAGCGCCTGGTCTAAGATTCCTCCTTCAC
GATTCAATGTCGGCGGGGTCTACCACCCCAATGGCCAGCGGGTAGGATCGATGCACGTTCGCGGTGGACACTTT
CTCGACGAAGACCCGGCTCTTTTCGATGCCTCATTTTTCAATATGAGTACTGAAGTTGCCAGTTGTATGGACCC
CCAGTACCGACTCATACTTGAAGTCGTTTATGAGGCGCTCGAAGCTGCGGGAATTCCTCTCGAACAGGTCTCCG
GCTCCAAGACTGGGGTTTTTGCAGGAACCATGTATCACGACTACCAAGGCTCCTTCCAGCGCCAACCAGAAGCC
CTTCCACGGTATTTCATAACAGGAAATGCTGGCACCATGCTCGCGAATCGCGTCTCCCACTTTTATGACCTTCG
TGGGCCCAGTGTCTCGATCGACACTGCCTGTTCCACAACCTTAACAGCCTTGCATCTTGCCATTCAGAGCTTGC
GAGCTGGAGAATCTGATATGGCGATTGTCGCTGGCGCGAACCTGCTACTTAATCCTGACGTCTTTACTACCATG
TCCAACCTTGGCTTCCTTTCGTCCGATGGGATTTCCTACTCATTTGACTCGAGAGCGGATGGCTATGGTCGCGG
AGAAGGAGTGGCTGCGATCGTCTTGAAGACTCTGCCCGATGCGGTGCGAGACGGAGACCCGATCCGCCTCATAG
TGCGCGAAACGGCAATCAACCAAGACGGCCGGACCCCAGCCATCAGCACGCCGAGCGGCGAGGCCCAGGAGTGC
CTGATCCAAGATTGCTATCAGAAGGCCCAGTTGGACCCAAAACAGACTTCGTACGTTGAGGCCCATGGGACGGG
AACCAGAGCAGGAGATCCGCTGGAGCTTGCAGTCATCTCGGCCGCGTTTCCGGGACAGCAGATACAGGTGGGCT
CCGTGAAAGCCAATATCGGGCATACAGAGGCTGTCAGTGGTCTGGCGAGTTTGATAAAGGTGGCTCTGGCTGTT
GAAAAGGGGGTTATCCCGCCTAATGCAAGGTTCCTCCAGCCGAGCAAGAAGTTGCTCAAGGACACTCATATCCA
GATTCCACTGTGTAGCCAATCATGGATACCAACCGATGGTGTCCGTCGCGCATCAATAAACAACTTCGGTTTCG
GAGGCGCAAATGCTCATGCAATCGTGGAGCAATATGGCCCGTTTGCAGAAACATCGATCTGCCCACCTAATGGT
TATTCTGGCAACTATGATGGCAATTTAGGAACGGATCAAGCGCATATATATGTGCTGAGTGCCAAGGATGAGAA
CAGTTGCATGAGAATGGTTTCAAGGCTGTGCGACTATGCTACCCACGCCAGACCAGCCGACGATTTGCAATTGC
TCGCGAATATAGCATACACGCTTGGTTCTCGTCGCTCGAACTTCCGATGGAAGGCAGTATGTACGGCACACAGC
CTCACGGGTCTTGCCCAGAATTTGGCGGGAGAAGGCATGCGGCCAAGCAAGTCAGCCGACCAAGTAAGACTGGG
ATGGGTGTTCACAGGCCAGGGAGCGCAATGGTTTGCAATGGGTCGTGAGTTGATTGAGATGTATCCTGTCTTTA
AAGAGGCCCTGCTGGAATGCGATGGATATATCAAGGAAATGGGGTCAACCTGGTCCATTATAGAGGAACTCAGT
CGCCCTGAAACGGAAAGTCGCGTTGATCAGGCAGAATTCAGTCTGCCATTGTCTACGGCTCTTCAAATTGCGCT
TGTTCGTCTGCTCTGGTCGTGGAACATCCAACCAGTAGCCGTCACTAGTCACTCCAGCGGAGAGGCAGCTGCAG
CGTACGCTATCGGGGCACTAACAGCCCGCTCGGCCATTGGAATAAGCTATATACGCGGTGCATTGACAGCAAGA
GACCGCCTGGCGTCGGTACATAAGGGGGGCATGTTGGCTGTCGGATTGAGCCGCAGTGAAGTGGGTATATACAT
CAGACAGGTTCCATTACAGAGTGAAGAATGCTTGGTGGTGGGGTGTGTCAACAGCCCGTCGAGTGTGACGGTCT
CGGGAGATTTGTCCGCCATTGCCAAGTTGGAGGAACTGCTCCATGCTGATCGTATATTTGCGAGACGGCTGAAA
GTCACCCAAGCCTTTCACTCCAGCCACATGAACTCGATGACAGATGCTTTCCGAGCCGGTCTTACAGAACTCTT
CGGAGCAGACCCCAGTGATGCAGCAAACGCCAGTAAAGATGTGATCTACGCTTCTCCCAGAACCGGGGCCCGCC
TGCACGACATGAATCGTCTTCGGGATCCTATACACTGGGTCGAATGCATGCTTCACCCGGTTGAGTTCGAATCA
GCATTCCGTCGAATGTGCCTGGACGAAAACGACCACATGCCAAAGGTCGATAGGGTCATTGAGATTGGACCTCA
CGGAGCGCTTGGAGGCCCGATCAAGCAGATCATGCAGCTTCCAGAGCTTGCCACGTGTGACATCCCTTATCTGT
CCTGTCTTTCTCGTGGGAAGAGCTCTCTGAGCACCCTTCGCCTTCTCGCATCAGAACTTATCCGGGCCGGATTT
CCTGTTGACTTGAATGCGATCAACTTTCCCCGCGGATGTGAAGCAGCTCGGGTCCAAGTGTTGTCTGATCTACC
GCCCTACCCTTGGAACCACGAGACCAGATACTGGAAAGAGCCGCGCATCAGCCAATCTGCCCGGCAGCGGAAGG
GCCCAGTCCACGATCTGATCGGATTGCAGGAGCCGTTGAACCTGCCGTTGGCGCGGTCATGGCACAATGTGCTT
CGTGTGTCAGATTTGCCATGGCTACGCGACCACGTCGTCGGCTCGCATATTGTTTTCCCTGGGGCTGGGTTCGT
GTGTATGGCAGTGATGGGAATCAGCACGCTCTGCTCGTCCGACCATGAATCTGACGACATCAGTTACATCCTAC
GCGACGTGAACTTTGCGCAGGCCCTGATTCTACCTGCGGACGGGGAAGAAGGAATAGATCTGCGCCTCACGATT
TGTGCTCCCGATCAGAGTCTGGGTTCACAGGACTGGCAAAGATTCTTAGTTCATTCGATCACTGCTGACAAGAA
TGACTGGACGGAACACTGTACGGGACTTGTTCGAGCAGAGATGGACCAGCCTCCCTCCAGTTTGTCGAACCAAC
AACGGATAGACCCACGGCCATGGAGCCGTAAAACGGCGCCGCAGGAGCTGTGGGACTCACTACATCGGGTGGGA
ATTCGTCATGGGCCCTTTTTTCGAAACATTACGTGCATCGAAAGCGACGGGCGAGGGTCATGGTGTACATTTGC
CATCGCGGACACGGCCTCCGCAATGCCACACGCCTACGAATCCCAGCACATTGTTCACCCAACCACACTAGACT
CTGCAGTTCAGGCAGCCTATACCACTCTTCCATTCGCTGGGAGCCGGATCAAATCTGCGATGGTCCCCGCTCGC
GTCGGCTGCATGAAGATTTCCTCCCGACTTGCAGATTTGGAGGCCAGGGACATGCTGCGCGCACAAGCGAAGAT
GCACAGCCAAAGTCCTTCCGCATTGGTAACCGATGTAGCAGTTTTTGATGAGGCAGATCCGGTTGGAGGGCCTG
TTATGGAGCTCGAAGGGCTGGTCTTTCAGTCTCTGGGGGCAAGTCTGGGCACTTCTGACCGGGACTCCACCGAC
CCCGGGAATACTTGCAGCTCCTGGCATTGGGCTCCAGACATCAGCTTAGTTAACCCCGGCTGGCTTGAAAAAAC
CCTGGGCACAGGTATTCAGGAGCACGAGATCAGCCTCATATTGGAGCTTCGACGGTGTTCGGTGCACTTCATTC
AAGAGGCCATGGAAAGTTTGAGCGTAGGCGATGTCGAGAGGCTGAGTGGTCATCTGGCCAAATTCTATGCGTGG
ATGCAGAAACAACTGGCGTGTGCCCAAAATGGCGAGCTGGGGCCAGAGAGCTCCAGCTGGACTCGGGATAGCGA
GCAGGCAAGATGCAGCCTCCGCTCTAGAGTGGTTGCTGGTAGCACCAACGGCGAAATGATCTGTCGCCTGGGCT
CCGTGCTCCCCGCTATCCTACGTCGGGAAGTTGATCCGTTGGAGGTGATGATGGATGGCCACCTGTTGTCCCGC
TACTATGTCGATGCCCTCAAGTGGAGTCGGTCCAACGCGCAAGCCAGCGAGCTCGTGCGCCTCTGCTGCCACAA
AAACCCGCGCGCTCGCATACTGGAAATCGGCGGAGGCACCGGGGGTTGCACCCAGCTGGTCGTGGACTCCTTGG
GCCCAAATCCGCCGGTAGGCCGCTATGACTTTACTGACGTCTCGGCCGGGTTTTTTGAAGCAGCCCGCAAGCGG
TTCGCGGGATGGCAGAATGTGATGGATTTTCGGAAGTTGGACATCGAGGACGATCCAGAAGCGCAGGGGTTTGT
GTGCGGATCCTACGACGTGGTGTTGGCTTGTCAGGTCCTGCATGCCACTTCTAACATGCAGCGCACATTGACTA
ATGTGCGCAAGCTGTTGAAGCCAGGAGGCAAACTCATTCTTGTCGAAACCACCAGAGACGAGCTTGACTTGTTT
TTCACTTTCGGGCTTCTGCCCGGCTGGTGGCTCAGCGAAGAACCAGAAAGACAGTCGACTCCGTCACTAAGCCC
TACGATGTGGCGCAGCATGCTGCACACTACTGGATTCAATGGTGTGGAAGTTGAGGCTCGTGACTGCGATAGCC
ACGAGTTCTATATGATTAGCACCATGATGTCCACGGCCGTACAGGCGACTCCGATGTCATGCTCGGTCAAATTG
CCTGAAGTGCTCTTGGTCTATGTTGACTCATCTACGCCCATGTCTTGGATATCAGATTTGCAGGGAGAGATTCG
CGGCAGGAATTGTTCCGTCACTTCGCTACAGGCACTTCGTCAAGTTCCTCCTACCGAGGGCCAAATATGCGTAT
TCCTTGGAGAGGTGGAACACTCCATGCTTGGTTCAGTCACCAACGACGACTTCACACTTTTGACCTCAATGCTA
CAGCTGGCTGGGGGAACTTTATGGGTCACCCAAGGAGCGACAATGAAGTCTGATGATCCCCTGAAGGCTCTACA
CCTCGGATTACTACGTACCATGCGTAATGAAAGCCATGGCAAGCGATTTGTCTCACTTGACCTCGACCCTTCGC
GTAATCCATGGACAGGCGATTCGCGCGATGCCATTGTCAGTGTTCTGGATTTAATTAGCATGTCAGATGAAAAG
GAGTTTGACTATGCAGAGCGGGATGGAGTTATCCATGTTCCTCGGGCATTTAGTGACTCCATCAATGGAGGCGA
GGAAGACGGGTATGCCTTGGAGCCATTCCAGGACAGCCAGCATCTCCTGCGACTAGATATACAGACTCCTGGGC
TCCTCGATTCCCTGCACTTCACAAAGCGCAATGTGGACACATATGAACCAGATAAATTACCGGACGACTGGGTA
GAGATTGAACCGAGGGCGTTTGGTCTTAACTTCCGTGACATCATGGTCGCGATGGGTCAATTGGAATCAAACGT
CATGGGCTTCGAATGCGCCGGCGTGGTTACAAGTCTCAGCGAGACAGCAAGAACAATTGCACCCGGGCTTGCGG
TCGGAGATCGGGTTTGCGCCCTCATGAACGGACACTGGGCGTCGAGGGTGACCACAAGCCGGACCAACGTGGTG
CGCATTCCAGAGACTCTTAGTTTCCCGCATGCTGCCTCCATCCCTCTGGCCTTCACAACAGCTTACATTTCACT
TTACACCGTTGCCCGCATTCTGCCAGGTGAAACGGTGTTGATCCATGCCGGGGCAGGAGGCGTAGGCCAGGCGG
CCATTATTCTTGCTCAATTAACCGGTGCTGAAGTCTTTACAACTGCTGGCAGTGAGACCAAGCGTAACCTTTTG
ATCGATAAATTCCACCTCGACCCTGATCATGTCTTCTCGAGCAGGGACTCCAGCTTCGTCGACGGTATCAAGAC
CCGCACCCGTGGCAAGGGGGTGGACGTGGTTTTGAACTCGCTAGCTGGGCCTCTCCTTCAGAAGAGCTTTGACT
GTCTGGCTAGGTTTGGTCGGTTTGTAGAAATCGGCAAGAAGGATCTTGAGCAGAATAGCCGACTCGACATGTCG
ACGTTCGTCCGCAATGTCTCCTTCTCCTCCGTTGATATTCTCTACTGGCAGCAAGCGAAGCCCGCTGAAATCTT
CCAGGCGATGTCCGAGGTCATCTTGCTGTGGGAGCGAACGGCAATCGGCCTGATTCATCCAATATCAGAGTATC
CTATGTCGGCCCTGGAGAAGGCCTTTCGCACTATGCAGAGCGGCCAGCACGTTGGGAAGATTGTTGTGACAGTA
GCCCCCGATGACGCGGTCCTCGTTCGTCAGGAACGAATGCCACTATTTCTGAAGCCTAACGTGTCGTATCTTGT
TGCTGGGGGCCTGGGTGGTATCGGACGGCGGATCTGCGAGTGGCTGGTCGATCGCGGGGCGCGATATCTCATCA
TTCTGTCTCGAACTGCTCGCGTGGACCCGGTCGTGACGAGTCTCCAAGAGCGGGGCTGCACCGTTTCTGTACAG
GCGTGTGATGTGGCCGATGAAAGCCAGCTTGAAGCGGCTCTCCAACAGTGTCGGGCGGAGGAAATGCCTCCGAT
TCGGGGCGTCATCCAAGGGGCAATGGTTCTCAAGGACGCCCTCGTCTCGCAAATGACGGCGGACGGGTTCCATG
CCGCCCTGCGGCCCAAGGTTCAGGGAAGTTGGAATCTGCACCGAATTGCATCGGACGTGGATTTCTTCGTGATG
CTCTCATCCTTGGTGGGTGTCATGGGAGGCGCAGGACAAGCCAACTACGCGGCTGCCGGAGCGTTTCAGGACGC
GCTCGCAGAGCACCGCATGGCTCACAACCAGCCAGCGGTCACCATCGACCTCGGAATGGTCCAGTCAATTGGGT
ATGTAGCAGAGACAGATTCTGCTGTGGCGGAACGACTCCAACGGATCGGCTATCAACCCTTGCACGAAGAGGAG
GTTCTGGACGTCCTCGAGCAAGCTATATCTCCTGTGTGTTCCCCTGCCGCACCCACACGGCCTGCTGTCATCGT
CACCGGCATCAACACCCGCCCAGGCCCTCACTGGGCACACGCCGACTGGATGCAAGAGGCTCGCTTTGCGGGGA
TCAAGTATCGTGATCCGTTGAGGGACAATCATGGAGCTTTGTCGCTGACCCCGGCGGAAGATGACAATCTTCAC
GCCAGGCTGAACCGTGCAATCAGCCAACAGGAGTCAATCGCCGTGATCATGGAGGCGATGAGCTGCAAGCTCAT
CTCAATGTTCGGCCTGACGGATAGCGAAATGTCCGCCACTCAGACATTGGCGGGGATCGGCGTGGACTCCCTGG
TCGCCATTGAGCTCCGGAACTGGATCACAGCTAAGTTCAATGTTGATATCTCAGTTTTCGAGTTGATGGAGGGC
CGAACGATCGCCAAAGTCGCGGAAGTGGTGCTGCAGAGATACAAAGCT
(SEQ ID NO: 12)
Aspergillus terreus LovG : ESTERASE. ACCESSION Q0C8M2
ATGCGCTACCAAGCATCTCCAGCACTGGCGAAGGCGCCCCGAGCGCTTCTTTGCATCCATGGGGCTGGCTGCTC
TCCCGCCATCTTCCGCGTGCAATTGTCTAAGCTCCGGGCTGCGCTGCGCGAAGACTTTGAATTCGTCTACGTGA
CAGCCCCGTTCCCTTCCTCTGCAGGGCCTGGGATTCTCCCCGTCTTCGCCGACCTGGGGCCATATTACTCATGG
TTTGAAAGCAGCAGCGGCAACAATAATAATGGGCCCTCCGTGGGCGAACGCCTCGCCGCCGTCCACGACCCCAT
CCGCCGCACCATTGTCGACTGGCAGACTCAACACCCCCACATCCCTATCGTGGGTGCTATCGGTTTCTCCGAAG
GTGCCCTGGTGACGACCTTGCTCCTCTGGCAGCAGCAGATGGGTCGCCTGCCCTGGTTGCCCCGGATGAGTGTC
GCGATGTTGATCTGCCCCTGGTATCAAGACGAGGCAAGCCAGTATATGAGGAACGAAGTGATGGAGAACCATGA
CGACGACCACGACAGCAAAGATACCGAGTGGCAGGAGGAACTGGTCATTCGGATACCGACATTACATCTGCAGG
GTCGCGATGATTTTGCGCTCGCAGGATCGAAGATGCTGGTGGCGCGCCATTTCTCCCCCCGAGAGGCGCAGGTA
TTGGAGTTTGCTGGGCAGCATCAGTTTCCCAATCGACCGCGCGACGTGTTGGAGGTTATTAATCGTTTTCGTAA
GCTGTGTGTGACGGTCCAGACATTGGAG
(SEQ ID NO: 13)
Aspergillus terreus NADPH--cytochrome P450 reductase. Accession QOCMMO
ATGGCTCAACTCGACACTCTCGACCTGGTGGTCCTGGTGGTGCTTTTGGTGGGTAGCGCCGCCTACTTCACCAA
GGGCACCTACTGGGCCGTTCCCAAGGACCCGTATGCCGCCTCCGGTCCCGCCATGAATGGTGGCGCCAAGGCGG
GCAAATCCAGGGACATCATTGAGAAAATGGAAGAGACTGGCAAGAACTGTGTGATTTTCTACGGCTCGCAGACC
GGTACCGCCGAGGATTATGCGTCGCGCCTGGCCAAGGAAGGCTCCCAGCGTTTCGGCCTCAAGACCATGGTCGC
AGATCTGGAAGACTACGATTATGAGAACCTGGACAAGTTCCCCGAGGACAAGGTTGCCTTCTTCGTCATGGCCA
CCTATGGTGAGGGTGAACCCACCGACAACGCCGTCGAGTTCTACCAGTTCATCTCGGGTGAGGACGTCGCGTTC
GAGAGCGGCGCCTCTGCCGACGACAAGCCCCTGTCCTCCCTCAAGTATGTCACTTTCGGTCTCGGTAACAACAC
CTATGAGCACTATCAGGCTATGGTTCGCAATCTGGATGCCGCTCTCACCAAGCTGGGTGCGCAGCGCATTGGAG
ATGCTGGTGAAGGTGATGACGGCGCTGGCACCATGGAAGAAGATTTCCTGGCCTGGAAAGAGCCCATGTGGACT
GCCCTGTCCGAGGCCATGAACCTTCAGGAGCGCGAAGCCGTCTATGAGCCGGTGTTCTCGGTCACGGAAGATGA
ATCCCTGTCCCCCGAAGACGAAGCCGTCTACCTCGGTGAGCCGACCAAGGGTCACCGTGACGGCACCCCCAGTG
GCCCGTATTCCGCTCACAACCCCTTCATCGCCCCCATCGTCGAGTCTCGTGAACTGTTCAACGTCAAGGACCGT
AATTGTCTGCACATGGAGATCAGCATCGCTGGTAGCAACCTTTCTTACCAGACTGGTGATCACATCGCGATTTG
GCCCACGAACGCTGGTGCCGAGGTGGACCGGTTCCTCCAGGTGTTTGGTCTTGAGAACAAGCGTCATTCCGTCA
TCAACATCAAGGGTATCGATGTGACCGCCAAGGTTCCCATTCCGACTCCCACCACGTATGATGCTGCTGTTCGC
TACTATATGGAAATTGCTGCGCCCGTCTCCCGTCAGTTTGTGGCTACCCTGGCTGCGTTTGCTCCCGATGAGGA
GACTAAGGCGGAAATCGTGCGTTTGGGTAGCGACAAGGACTACTTCCACGAGAAAATCAGCAACCAGTGCTTCA
CCATCGCTCAGGCTCTTCAGAGTGTCACCTCCAAGCCCTTCTCGGCTGTCCCGTTCTCTCTGCTTATCGAGGGT
CTCAATAAGCTCCAGCCCCGTTACTACTCCATCTCTTCCTCCTCCATGGTCCAGAAGGATAAGATCAGCATTAC
TGCCGTCGTGGAATCCACTCGCTTGCCTGGTGCCGCCCACCTTGTCAAGGGTGTCACGACCAACTATCTCCTTG
CCCTGAAGCAAAAGCAGAATGGGGATCCGTCTCCCGACCCTCACGGCTTAACTTATACTATCACTGGGCCCCGT
AACAAGTACGACGGAATCCACGTTCCCGTTCACGTCCGCCACTCCAATTTCAAGCTCCCCTCTGATCCCTCTCG
GCCCATTATCATGGTTGGCCCTGGTACCGGTGTGGCTCCCTTCCGTGGATTCATCCAGGAGCGTGCCGCCTTGG
CCGCCAAGGGTGAGAACGTCGGTCCCACCGTGTTGTTCTTTGGATGCCGCAGGCGCGATGAGGACTTTATGTAC
GCAGATGAATTCAAGACCTACCAGGAACAGCTTGGGGACAAGCTTCAGATCATTACTGCGTTTTCTCGTGAAAC
TTCCCAGAAGGTGTATGTTCAGCACAGACTGCGTGAACACTCCGATCTGGTGAGCAGCCTCCTGAAGCAGAAGG
CTAACTTTTACGTCTGCGGTGACGCCGCCAACATGGCGCGTGAAGTCAACCTTGTGCTTGGCCAGATCATCGCG
CAACAGCGGGGTCTCCCGGCTGAACGGGCCGAGGAAATGGTGAAGCACATGCGCAGCAGCGGCAGCTACCAGGA
GGACGTGTGGTCA
(SEQ ID NO: 14)

1. A method of making simvastatin comprising the steps of:
(1) combining a microbial host organism with a 2,2-dimethylbutyryl donor compound, wherein:
(a) the microbial host organism comprises genes expressing LovA (SEQ ID NO: 1), LovB (SEQ ID NO: 2), LovC (SEQ ID NO: 3), LovD (SEQ ID NO: 4), LovG (SEQ ID NO: 5), and cytochrome P450 oxidoreductase (SEQ ID NO: 1);
(b) two or more genes in (a) are not naturally expressed by the microbial host organism; and
(c) the microbial host organism produces monacolin J; and
(2) allowing LovD to transfer the dimethylbutyryl_group from the 2,2-dimethylbutyryl donor compound to regioselectively acylate the C8 hydroxyl group of monacolin J;
so that simvastatin is made.

2. The method of claim 1, wherein the simvastatin is formed in a one-pot fermentation process.

3. The method of claim 1, wherein the microbial host organism is grown under at least one of the following conditions:
(a) at a temperature between 30-40° C.;
(b) for a time period between at least 4 to at least 48 hours;
(c) at a pH between 7-8; or
(d) in a fermentation media comprising YPD, LYPD, YNB, HC or YC media.

4. The method of claim 1, further comprising purifying the simvastatin made by the method by at least one purification step comprising:
(a) lysis of cells of an isolated organism present in the combination;
(b) centrifugation;
(c) precipitation of a free acid form of simvastatin;
(d) conversion of a free acid form of simvastatin to a simvastatin salt;
(e) filtration; or
(f) high performance liquid chromatography (HPLC).

5. The method of claim 1, wherein the organism is Saccharomyces cerevisiae.

6. The method of claim 1, wherein the organism is Escherichia coli, Monascus ruber, Monascus purpureus, Monascus pilosus, Monascus vitreus, Monascus pubigerus, Candida cariosilognicola, Aspergillus oryzea, Doratomyces stemonitis, Paecilomyces virioti, Penicillum citrinum, Penicillin chrysogenum, Scopulariopsis brevicaulis or Trichoderma viride.

7. The method of claim 1, wherein the 2,2-dimethylbutyryl donor compound is selected to possess at least one of the following properties:
(a) is a butyrlyl-thioester, a N-acetylcysteamine thioester or a methyl-thioglycolate thioester;
(b) comprises medium chain length (C3-C6) acyl group moieties;
(c) is able to cross the cellular membranes of Escherichia coli or Aspergillus terreus cells growing within a fermentation media; or
(d) is selected from the group consisting of α-dimethylbutyryl-S-methyl-mercaptopropionate (DMB-S-MMP), dimethylbutyryl-S-ethyl mercaptopropionate (DMB-S-EMP) and dimethylbutyryl-S-methyl thioglycolate (DMB-S-MTG) and dimethylbutyryl-S-methyl mercaptobutyrate (DMB-S-MMB).

8. The method of claim 1, wherein one or more genes expressing LovA (SEQ ID NO: 1), LovB (SEQ ID NO: 2), LovC (SEQ ID NO: 3), LovD (SEQ ID NO: 4), LovG (SEQ ID NO: 5), and cytochrome P450 oxidoreductase (SEQ ID NO: 6) is codon optimized.

9. The method of claim 1, wherein expressing LovA (SEQ ID NO: 1), LovB (SEQ ID NO: 2), LovC (SEQ ID NO: 3), LovD (SEQ ID NO: 4), LovG (SEQ ID NO: 5), and cytochrome P450 oxidoreductase (SEQ ID NO: 6) are expressed episomally in at least two separate vectors.

10. A composition of matter comprising a microbial host organism wherein:
the microbial host organism comprises heterologous genes expressing LovA (SEQ ID NO: 1), LovB (SEQ ID NO: 2), LovC (SEQ ID NO: 3), LovG (SEQ ID NO: 5), and cytochrome P450 oxidoreductase (SEQ ID NO: 6);
the microbial host organism can produce monacolin J during fermentation.

11. The composition of claim 10, further comprising a culture media in which the microbial host organism produces monacolin J during fermentation.

12. The composition of claim 10, further comprising LovD (SEQ ID NO: 4).

13. The composition of claim 10, further comprising monacolin J.

14. The composition of claim 10, further comprising simvastatin.

15. The composition of claim 10, further comprising a 2,2-dimethylbutyryl donor compound.

16. A method of making monacolin J comprising the steps of:
(1) combining a first microbial host organism with a growth medium, wherein:
(a) the microbial host organism comprises genes expressing LovA (SEQ ID NO: 1), LovB (SEQ ID NO: 2), LovC (SEQ ID NO: 3), LovG (SEQ ID NO: 5), and cytochrome P450 oxidoreductase (SEQ ID NO: 6); and
(b) one or more genes in (a) is not naturally expressed by the microbial host organism;
so that monacolin J is made.

17. The method of claim 16, wherein monacolin j is made in concentrations of at least 10 mg/L.

18. The method of claim 16, wherein the organism is Saccharomyces cerevisiae.

19. The method of claim 16, further comprising converting the Monacolin J to simvastatin by coculturing the first microbial host organism with:
(a) a second microbial host organism comprising a gene expressing LovD (SEQ ID NO: 4); and
(b) a 2,2-dimethylbutyryl donor compound.

20. The method of claim 19, further comprising purifying the simvastatin made by the method by at least one purification step comprising:
(a) lysis of cells of an isolated organism present in the combination;
(b) centrifugation;
(c) precipitation of a free acid form of simvastatin;
(d) conversion of a free acid form of simvastatin to a simvastatin salt;
(e) filtration; or
(f) high performance liquid chromatography (HPLC).


SEQUENCE LISTING (33 sequences)

SEQ ID NO: 1 - protein, 528 aa, Aspergillus terreus
MTVDALTQPHHLLSLAWNDTQQHGSWFAPLVTTSAGLLCLLLYLCSSGRRSDLPVFNPKTWWELTTMRAKRDFDANAPSWIESWFSQNDKPIRFIVDSGYCTILPSSMADEFRKMKELCMYKFLGTDFHSHLPGFDGFKEVTRDAHLITKVVMNQFQTQAPKYVKPLANEASGIITDIFGDSNEWHTVPVYNQCLDLVTRTVTFIMVGSKLAHNEEWLDIAKHHAVTMAIQARQLRLWPVILRPLVHWLEPQGAKLRAQVRRARQLLDPIIQERRAERDACRAKGIEPPRYVDSIQWFEDTAKGKWYDAAGAQLAMDFAGIYGTSDLLIGGLVDIVRHPHLLEPLRDEIRTVIGQGGWTPASLYKLKLLDSCLKESQRVKPVECATMRSYALQDVTFSNGTFIPKGELVAVAADRMSNPEVWPEPAKYDPYRYMRLREDPAKAFSAQLENTNGDHIGFGWHPRACPGRFFASKEIKMMLAYLLIRYDWKVVPDEPLQYYRHSFSVRIHPTTKLMMRRRDEDIRLPGSL

SEQ ID NO: 2 - protein, 3038 aa, Aspergillus terreus
MAQSMYPNEPIVVVGSGCRFPGDANTPSKLWELLQHPRDVQSRIPKERFDVDTFYHPDGKHHGRTNAPYAYVLQDDLGAFDAAFFNIQAGEAESMDPQHRLLLETVYEAVTNAGMRIQDLQGTSTAVYVGVMTHDYETVSTRDLESIPTYSATGVAVSVASNRISYFFDWHGPSMTIDTACSSSLVAVHLAVQQLRTGQSSMAIAAGANLILGPMTFVLESKLSMLSPSGRSRMWDAGADGYARGEAVCSVVLKTLSQALRDGDTIECVIRETGVNQDGRTTGITMPNHSAQEALIKATYAQAGLDITKAEDRCQFFEAHGTGTPAGDPQEAEAIATAFFGHEQVARSDGNERAPLFVGSAKTVVGHTEGTAGLAGLMKASFAVRHGVIPPNLLFDKISPRVAPFYKNLRIPTEATQWPALPPGQPRRASVNSFGFGGTNAHAIIEEYMEPEQNQLRVSNNEDCPPMTGVLSLPLVLSAKSQRSLKIMMEEMLQFLQSHPEIHLHDLTWSLLRKRSVLPFRRAIVGHSHETIRRALEDAIEDGIVSSDFTTEVRGQPSVLGIFTGQGAQWPGMLKNLIEASPYVRNIVRELDDSLQSLPEKYRPSWTLLDQFMLEGEASNVQYATFSQPLCCAVQIVLVRLLEAARIRFTAVVGHSSGEIACAFAAGLISASLAIRIAYLRGVVSAGGARGTPGAMLAAGMSFEEAQEICELDAFEGRICVAASNSPDSVTFSGDANAIDHLKGMLEDESTFARLLKVDTAYHSHHMLPCADPYMQALEECGCAVADAGSPAGSVPWYSSVDAENRQMAARDVTAKYWKDNLVSPVLFSHAVQRAVVTHKALDIGIEVGCHPALKSPCVATIKDVLSGVDLAYTGCLERGKNDLDSFSRALAYLWERFGASSFDADEFMRAVAPDRPCMSVSKLLPAYPWDRSRRYWVESRATRHHLRGPKPHLLLGKLSEYSTPLSFQWLNFVRPRDIEWLDGHALQGQTVFPAAGYIVMAMEAALMIAGTHAKQVKLLEILDMSIDKAVIFDDEDSLVELNLTADVSRNAGEAGSMTISFKIDSCLSKEGNLSLSAKGQLALTIEDVNPRTTSASDQHHLPPPEEEHPHMNRVNINAFYHELGLMGYNYSKDFRRLHNMQRADLRASGTLDFIPLMDEGNGCPLLLHPASLDVAFQTVIGAYSSPGDRRLRCLYVPTHVDRITLVPSLCLATAESGCEKVAFNTINTYDKGDYLSGDIVVFDAEQTTLFQVENITFKPFSPPDASTDHAMFARWSWGPLTPDSLLDNPEYWATAQDKEAIPIIERIVYFYIRSFLSQLTLEERQQAAFHLQKQIEWLEQVLASAKEGRHLWYDPGWENDTEAQIEHLCTANSYHPHVRLVQRVGQHLLPTVRSNGNPFDLLDHDGLLTEFYTNTLSFGPALHYARELVAQIAHRYQSMDILEIGAGTGGATKYVLATPQLGFNSYTYTDISTGFFEQAREQFAPFEDRMVFEPLDIRRSPAEQGFEPHAYDLIIASNVLHATPDLEKTMAHARSLLKPGGQMVILEITHKEHTRLGFIFGLFADWWAGVDDGRCTEPFVSFDRWDAILKRVGFSGVDSRTTDRDANLFPTSVFSTHAIDATVEYLDAPLASSGTVKDSYPPLVVVGGQTPQSQRLLNDIKAIMPPRPLQTYKRLVDLLDAEELPMKSTFVMLTELDEELFAGLTEETFEATKLLLTYASNTVWLTENAWVQHPHQASTIGMLRSIRREHPDLGVHVLDVDAVETFDATFLVEQVLRLEEHTDELASSTTWTQEPEVSWCKGRPWIPRLKRDLARNNRMNSSRRPIYEMIDSSRAPVALQTARDSSSYFLESAETWFVPESVQQMETKTIYVHFSCPHALRVGQLGFFYLVQGHVQEGNREVPVVALAERNASIVHVRPDYIYTEADNNLSEGGGSLMVTVLAAAVLAETVISTAKCLGVTDSILVLNPPSICGQMLLHAGEEIGLQVHLATTSGNRSSVSAGDAKSWLTLHARDTDWHLRRVLPRGVQALVDLSADQSCEGLTQRMMKVLMPGCAHYRAADLFTDTVSTELHSGSRHQASLPAAYWEHVVSLARQGLPSVSEGWEVMPCTQFAAHADKTRPDLSTVISWPRESDEATLPTRVRSIDAETLFAADKTYLLVGLTGDLGRSLGRWMVQHGACHIVLTSRNPQVNPKWLAHVEELGGRVTVLSMDVTSQNSVEAGLAKLKDLHLPPVGGIAFGPLVLQDVMLNNMELPMMEMVLNPKVEGVRILHEKFSDPTSSNPLDFFVMFSSIVAVMGNPGQANYSAANCYLQALAQQRVASGLAASTIDIGAVYGVGFVTRAELEEDFNAIRFMFDSVEEHELHTLFAEAVVAGRRAVHQQEQQRKFATVLDMADLELTTGIPPLDPALKDRITFFDDPRIGNLKIPEYRGAKAGEGAAGSKGSVKEQLLQATNLDQVRQIVIDGLSAKLQVTLQIPDGESVHPTIPLIDQGVDSLGAVTVGTWFSKQLYLDLPLLKVLGGASITDLANEAAARLPPSSIPLVAATDGGAESTDNTSENEVSGREDTDLSAAATITEPSSADEDDTEPGDEDVPRSHHPLSLGQEYSWRIQQGAEDPTVFNNTIGMFMKGSIDLKRLYKALRAVLRRHEIFRTGFANVDENGMAQLVFGQTKNKVQTIQVSDRAGAEEGYRQLVQTRYNPAAGDTLRLVDFFWGQDDHLLVVAYHRLVGDGSTTENIFVEAGQLYDGTSLSPHVPQFADLAARQRAMLEDGRMEEDLAYWKKMHYRPSSIPVLPLMRPLVGNSSRSDTPNFQHCGPWQQHEAVARLDPMVAFRIKERSRKHKATPMQFYLAAYQVLLARLTDSTDLTVGLADTNRATVDEMAAMGFFANLLPLRFRDFRPHITFGEHLIATRDLVREALQHARVPYGVLLDQLGLEVPVPTSNQPAPLFQAVFDYKQGQAESGTIGGAKITEVIATRERTPYDVVLEMSDDPTKDPLLTAKLQSSRYEAHHPQAFLESYMSLLSMFSMNPALKLA

SEQ ID NO: 3 - protein, 363 aa, Aspergillus terreus
MGDQPFIPPPQQTALTVNDHDEVTVWNAAPCPMLPRDQVYVRVEAVAINPSDTKMRGQFATPWAFLGTDYAGTVVAVGSDVTHIQVGDRVYGAQNEMCPRTPDQGAFSQYTVTRGRVWAKIPKGLSFEQAAALPAGISTAGLAMKLLGLPLPSPSADQPPTHSKPVYVLVYGGSTATATVTMQMLRLSGYIPIATCSPHNFDLAKSRGAEEVFDYRAPNLAQTIRTYTKNNLRYALDCITNVESTTFCFAAIGRAGGHYVSLNPFPEHAATRKMVTTDWTLGPTIFGEGSTWPAPYGRPGSEEERQFGEDLWRIAGQLVEDGRLVHHPLRVVQGGFDHIKQGMELVRKGELSGEKLVVRLEGP

SEQ ID NO: 4 - protein, 413 aa, Aspergillus terreus
MGSIIDAAAAADPVVLMETAFRKAVKSRQIPGAVIMARDCSGNLNYTRCFGARTVRRDECNQLPPLQVDTPCRLASATKLLTTIMALQCMERGLVDLDETVDRLLPDLSAMPVLEGFDDAGNARLRERRGKITLRHLLTHTSGLSYVFLHPLLREYMAQGHLQSAEKFGIQSRLAPPAVNDPGAEWIYGANLDWAGKLVERATGLDLEQYLQENICAPLGITDMTFKLQQRPDMLARRADQTHRNSADGRLRYDDSVYFRADGEECFGGQGVFSGPGSYMKVLHSLLKRDGLLLQPQTVDLMFQPALEPRLEEQMNQHMDASPHINYGGPMPMVLRRSFGLGGIIALEDLDGENWRRKGSLTFGGGPNIVWQIDPKAGLCTLAFFQLEPWNDPVCRDLTRTFEHAIYAQYQQG

SEQ ID NO: 5 - protein, 2532 aa, Aspergillus terreus
MTPLDAPGAPAPIAMVGMGCRFGGGATDPQKLWKLLEEGGSAWSKIPPSRFNVGGVYHPNGQRVGSMHVRGGHFLDEDPALFDASFFNMSTEVASCMDPQYRLILEVVYEALEAAGIPLEQVSGSKTGVFAGTMYHDYQGSFQRQPEALPRYFITGNAGTMLANRVSHFYDLRGPSVSIDTACSTTLTALHLAIQSLRAGESDMAIVAGANLLLNPDVFTTMSNLGFLSSDGISYSFDSRADGYGRGEGVAAIVLKTLPDAVRDGDPIRLIVRETAINQDGRTPAISTPSGEAQECLIQDCYQKAQLDPKQTSYVEAHGTGTRAGDPLELAVISAAFPGQQIQVGSVKANIGHTEAVSGLASLIKVALAVEKGVIPPNARFLQPSKKLLKDTHIQIPLCSQSWIPTDGVRRASINNFGFGGANAHAIVEQYGPFAETSICPPNGYSGNYDGNLGTDQAHIYVLSAKDENSCMRMVSRLCDYATHARPADDLQLLANIAYTLGSRRSNFRWKAVCTAHSLTGLAQNLAGEGMRPSKSADQVRLGWVFTGQGAQWFAMGRELIEMYPVFKEALLECDGYIKEMGSTWSIIEELSRPETESRVDQAEFSLPLSTALQIALVRLLWSWNIQPVAVTSHSSGEAAAAYAIGALTARSAIGISYIRGALTARDRLASVHKGGMLAVGLSRSEVGIYIRQVPLQSEECLVVGCVNSPSSVTVSGDLSAIAKLEELLHADRIFARRLKVTQAFHSSHMNSMTDAFRAGLTELFGADPSDAANASKDVIYASPRTGARLHDMNRLRDPIHWVECMLHPVEFESAFRRMCLDENDHMPKVDRVIEIGPHGALGGPIKQIMQLPELATCDIPYLSCLSRGKSSLSTLRLLASELIRAGFPVDLNAINFPRGCEAARVQVLSDLPPYPWNHETRYWKEPRISQSARQRKGPVHDLIGLQEPLNLPLARSWHNVLRVSDLPWLRDHVVGSHIVFPGAGFVCMAVMGISTLCSSDHESDDISYILRDVNFAQALILPADGEEGIDLRLTICAPDQSLGSQDWQRFLVHSITADKNDWTEHCTGLVRAEMDQPPSSLSNQQRIDPRPWSRKTAPQELWDSLHRVGIRHGPFFRNITCIESDGRGSWCTFAIADTASAMPHAYESQHIVHPTTLDSAVQAAYTTLPFAGSRIKSAMVPARVGCMKISSRLADLEARDMLRAQAKMHSQSPSALVTDVAVFDEADPVGGPVMELEGLVFQSLGASLGTSDRDSTDPGNTCSSWHWAPDISLVNPGWLEKTLGTGIQEHEISLILELRRCSVHFIQEAMESLSVGDVERLSGHLAKFYAWMQKQLACAQNGELGPESSSWTRDSEQARCSLRSRVVAGSTNGEMICRLGSVLPAILRREVDPLEVMMDGHLLSRYYVDALKWSRSNAQASELVRLCCHKNPRARILEIGGGTGGCTQLVVDSLGPNPPVGRYDFTDVSAGFFEAARKRFAGWQNVMDFRKLDIEDDPEAQGFVCGSYDVVLACQVLHATSNMQRTLTNVRKLLKPGGKLILVETTRDELDLFFTFGLLPGWWLSEEPERQSTPSLSPTMWRSMLHTTGFNGVEVEARDCDSHEFYMISTMMSTAVQATPMSCSVKLPEVLLVYVDSSTPMSWISDLQGEIRGRNCSVTSLQALRQVPPTEGQICVFLGEVEHSMLGSVTNDDFTLLTSMLQLAGGTLWVTQGATMKSDDPLKALHLGLLRTMRNESHGKRFVSLDLDPSRNPWTGDSRDAIVSVLDLISMSDEKEFDYAERDGVIHVPRAFSDSINGGEEDGYALEPFQDSQHLLRLDIQTPGLLDSLHFTKRNVDTYEPDKLPDDWVEIEPRAFGLNFRDIMVAMGQLESNVMGFECAGVVTSLSETARTIAPGLAVGDRVCALMNGHWASRVTTSRTNVVRIPETLSFPHAASIPLAFTTAYISLYTVARILPGETVLIHAGAGGVGQAAIILAQLTGAEVFTTAGSETKRNLLIDKFHLDPDHVFSSRDSSFVDGIKTRTRGKGVDVVLNSLAGPLLQKSFDCLARFGRFVEIGKKDLEQNSRLDMSTFVRNVSFSSVDILYWQQAKPAEIFQAMSEVILLWERTAIGLIHPISEYPMSALEKAFRTMQSGQHVGKIVVTVAPDDAVLVRQERMPLFLKPNVSYLVAGGLGGIGRRICEWLVDRGARYLIILSRTARVDPVVTSLQERGCTVSVQACDVADESQLEAALQQCRAEEMPPIRGVIQGAMVLKDALVSQMTADGFHAALRPKVQGSWNLHRIASDVDFFVMLSSLVGVMGGAGQANYAAAGAFQDALAEHRMAHNQPAVTIDLGMVQSIGYVAETDSAVAERLQRIGYQPLHEEEVLDVLEQAISPVCSPAAPTRPAVIVTGINTRPGPHWAHADWMQEARFAGIKYRDPLRDNHGALSLTPAEDDNLHARLNRAISQQESIAVIMEAMSCKLISMFGLTDSEMSATQTLAGIGVDSLVAIELRNWITAKFNVDISVFELMEGRTIAKVAEVVLQRYKA

SEQ ID NO: 6 - protein, 256 aa, Aspergillus terreus
MRYQASPALAKAPRALLCIHGAGCSPAIFRVQLSKLRAALREDFEFVYVTAPFPSSAGPGILPVFADLGPYYSWFESSSGNNNNGPSVGERLAAVHDPIRRTIVDWQTQHPHIPIVGAIGFSEGALVTTLLLWQQQMGRLPWLPRMSVAMLICPWYQDEASQYMRNEVMENHDDDHDSKDTEWQEELVIRIPTLHLQGRDDFALAGSKMLVARHFSPREAQVLEFAGQHQFPNRPRDVLEVINRFRKLCVTVQTLE

SEQ ID NO: 7 - protein, 695 aa, Aspergillus terreus
MAQLDTLDLVVLVVLLVGSAAYFTKGTYWAVPKDPYAASGPAMNGGAKAGKSRDIIEKMEETGKNCVIFYGSQTGTAEDYASRLAKEGSQRFGLKTMVADLEDYDYENLDKFPEDKVAFFVMATYGEGEPTDNAVEFYQFISGEDVAFESGASADDKPLSSLKYVTFGLGNNTYEHYQAMVRNLDAALTKLGAQRIGDAGEGDDGAGTMEEDFLAWKEPMWTALSEAMNLQEREAVYEPVFSVTEDESLSPEDEAVYLGEPTKGHRDGTPSGPYSAHNPFIAPIVESRELFNVKDRNCLHMEISIAGSNLSYQTGDHIAIWPTNAGAEVDRFLQVFGLENKRHSVINIKGIDVTAKVPIPTPTTYDAAVRYYMEIAAPVSRQFVATLAAFAPDEETKAEIVRLGSDKDYFHEKISNQCFTIAQALQSVTSKPFSAVPFSLLIEGLNKLQPRYYSISSSSMVQKDKISITAVVESTRLPGAAHLVKGVTTNYLLALKQKQNGDPSPDPHGLTYTITGPRNKYDGIHVPVHVRHSNFKLPSDPSRPIIMVGPGTGVAPFRGFIQERAALAAKGENVGPTVLFFGCRRRDEDFMYADEFKTYQEQLGDKLQIITAFSRETSQKVYVQHRLREHSDLVSSLLKQKANFYVCGDAANMAREVNLVLGQIIAQQRGLPAERAEEMVKHMRSSGSYQEDVWS

SEQ ID NO: 8 - DNA, 1584 bp, Aspergillus terreus
atgactgtcgacgcgctcacacagccgcaccaccttctgtcgctggcttggaatgacacgcagcaacatggctcgtggtttgcgcccttggtcactaccagtgcggggctactatgccttcttctttacctgtgctcgagtggccggagatctgatctgccggtgttcaatccgaaaacatggtgggaactgacgaccatgagggccaaacgggattttgatgcgaatgcaccgtcatggattgagagctggttctcgcaaaatgataagcccattcggttcatagtcgactctgggtactgcaccattcttccctcctctatggccgatgagtttcgcaagatgaaagagctctgtatgtacaagttcttgggcacggactttcactctcatcttcccggattcgatggattcaaggaagtcacgagggatgcacatctcatcaccaaggtggttatgaaccagttccagacccaagctcccaagtacgtcaagcctcttgccaatgaagccagcgggattatcacggatatttttggcgacagcaatgaatggcacacagtgcctgtctataaccagtgtctggacttagtgacccgaacagtgacttttattatggtcgggagcaagttagcccataatgaggagtggcttgacatcgccaagcaccacgcggtgacgatggcaattcaagcgcgccagctgcgcctctggcccgtcattctgcgcccccttgtacattggctcgagccccagggagccaaactccgggcgcaggttcgacgagcccggcaacttctcgatcccattatccaggagcgacgtgcggaaagagatgcctgccgggcaaagggcattgagccgcctcgctacgtagactcgatccagtggttcgaggatactgccaaggggaaatggtacgatgcagccggggcgcaactggccatggactttgctggtatctacggaacctccgacctgctgatcggtgggttggtggacatcgtccgacatccccatctccttgagcccctccgtgatgagatccggacggtcatcggccaagggggttggacacctgcctcgctgtacaagctcaaactgctggatagttgtctcaaggagtcacagcgcgtcaagcccgtcgaatgtgccaccatgcgcagctatgcattgcaggatgtgactttctccaatggaacctttatcccaaaaggagagctggtggcggtagctgccgaccgcatgagcaaccccgaggtctggccagagccggcaaaatacgatccttaccggtatatgcgcctgcgagaggacccggctaaagcgttcagtgcccaactggagaacaccaacggggaccacatcggcttcggttggcatccacgggcttgccccggccggttctttgcctctaaggagatcaagatgatgttagcctacttgctcatacgatacgactggaaggtggtccccgacgaaccgttgcagtactaccgccattctttcagcgtgcgcattcatcccaccacgaagctcatgatgcgccggcgcgacgaggatatccgccttcctggttcacta

SEQ ID NO: 9 - DNA, 9114 bp, Aspergillus terreus
atggctcaatctatgtatcctaatgagcctattgtcgtggtcggcagtggttgtcgcttccctggtgacgccaacacaccctccaagctctgggagctactccagcatcctcgcgatgtgcagagtcgaatccccaaagaacgatttgacgtcgacacattttatcacccggacgggaagcaccacgggcgaacaaatgcaccctacgcctatgttctccaagacgatctgggcgccttcgatgcggccttcttcaatatccaggctggagaggccgagagtatggacccccagcaccggctgttgctggagacggtgtacgaggccgtaacgaatgctggaatgcgtatccaggatctgcagggaacttcgactgctgtttacgtcggggtgatgacgcacgactatgagactgtctcaacccgcgacctggagagcatccccacctactcggcgacgggtgtcgcggtcagtgttgcgtccaaccgcatctcgtatttttttgactggcatggaccaagtatgacgatcgatacggcatgcagctcgtcgttggttgccgttcatctggcggtgcaacagctacggacgggtcaaagctccatggcaattgctgcgggtgcgaatctgattctggggcccatgacattcgtccttgaaagcaaattgagcatgctatccccctcgggtcgatcccgcatgtgggacgccggagctgacggctatgccagaggcgaagctgtttgctctgtagtgttgaagacattgagtcaagccttgcgcgatggggacacgattgaatgtgtcatccgagaaactggggtgaatcaagatggccgaacgaccggaattacgatgccgaaccatagtgctcaggaggcactcatcaaggctacctacgcccaggctggccttgacatcaccaaggccgaggacaggtgccaattcttcgaggctcatgggactggtactccggccggagatccccaggaggcggaggccattgcaacagccttcttcggccacgagcaggtagcacgcagcgacggaaacgagagggcccctctgttcgtgggcagtgcgaaaactgttgtcgggcacaccgagggcacggccggtctggctggtctcatgaaggcgtcgttcgctgtccgccatggggtaatcccccccaacctgctgttcgacaaaatcagcccgcgagtcgccccattctataaaaacctgaggattccgacagaagctacccaatggccagctctcccacccggacaaccgcgccgcgccagtgtcaactcctttggattcggcggcacgaatgcgcatgccattattgaggaatacatggagccagagcaaaaccagctgcgagtctcgaataatgaggactgcccacccatgaccggtgtcctgagtttacccttagtcctctcggcgaagtcccagcgctccttaaagataatgatggaggagatgctgcaattccttcagtctcaccccgagatacacttgcacgacctcacctggtccttactgcgcaagcggtcagttctacccttccgccgggctattgtcggccatagtcatgaaaccatccgccgggctttggaggatgccatcgaggatggtattgtgtcgagcgacttcactacggaggtcagaggccagccatcggtgttgggaatcttcaccgggcagggggcgcagtggccggggatgttaaagaatctgatagaggcatcgccatatgtgcggaacatagtgagggagctggacgactccctgcagagcttgccggaaaaataccggccctcgtggacgctactggaccagttcatgctagaaggagaggcctccaacgtccaatatgctactttctcccagccattatgctgcgcggtgcaaattgtcctggtccgtctccttgaagccgcgagaatacgattcacggctgttgttggacatagctccggcgaaattgcttgcgcctttgctgccgggctcatcagtgcctcgttggcgattcggattgcttacttacgtggagtcgtctcggcagggggcgccagaggcacaccgggagccatgttggccgccgggatgtcctttgaggaagcacaagagatctgcgagttggatgcctttgagggccgcatctgcgtggctgccagcaattccccagacagtgtaactttctctggcgacgcgaacgcaattgatcacctgaagggcatgttggaggatgagtccacttttgcgagactgctcaaggtcgatacagcgtaccactcgcatcatatgcttccatgtgcagacccatatatgcaagccctagaagagtgtggttgtgctgttgccgatgcaggttccccagccggaagtgtaccctggtattcgtccgtggacgccgagaacaggcaaatggcagcaagagacgtgaccgccaagtactggaaagataacttagtatctccggtgctattctcccacgcagtgcagcgggcagtcgtcacgcacaaggcgctggatatcgggattgaagtgggctgtcacccagctctcaagagcccatgcgtcgccaccatcaaggatgtcctatctggggttgacctggcgtatacaggttgcttggagcgaggaaagaatgatctcgattcattctctcgagcactggcatatctctgggaaaggtttggtgcctccagtttcgatgcggacgagttcatgcgtgcagtcgcgcctgatcggccctgtatgagtgtgtcgaagctcctaccggcctatccatgggaccgctctcgtcgctactgggtggaatcccgagcaactcgccaccatcttcgagggcccaagccccatcttctattaggaaagctctccgaatacagcactccgctaagcttccagtggctgaattttgtgcgcccacgagacattgaatggcttgatggacatgcattgcaaggccagactgtcttccctgcggccggctatatcgtcatggcaatggaagcagccttaatgattgctggcacccacgcaaagcaggtcaagttactggagatcttggatatgagcattgacaaggcggtgatatttgacgacgaagacagcttggttgagctcaacctgacagctgacgtgtctcgcaacgccggcgaagcaggttcaatgaccataagcttcaagatcgattcctgtctatcgaaggagggtaacctatccctatcagccaagggccaactggccctaacgatagaagatgtcaatcccaggacgacttccgctagcgaccagcaccatcttcccccgccagaagaggaacatcctcatatgaaccgtgtcaacatcaatgctttctaccacgagctggggttgatggggtacaactacagtaaggacttccggcgtctccataacatgcaacgagcagatcttcgagccagcggcaccttagacttcattcctctgatggacgagggtaatggctgtcctctcctgctgcatcctgcatcattggacgtcgccttccagactgtcatcggcgcatactcctccccaggtgatcggcgtctacgctgtctgtatgtacccactcacgttgatcgcatcacacttgtcccatccctttgcctggcaacggctgagtccggatgcgagaaggttgccttcaatactatcaatacgtacgacaagggagactacttgagcggtgacattgtggtgtttgacgcggagcagaccaccctgttccaggttgaaaatattacttttaagcccttttcacccccggatgcttcaactgaccatgcgatgtttgcccgatggagctggggtccgttgactccggactcgctgctggataacccggagtattgggccaccgcgcaggacaaggaggcgattcctattatcgaacgcatcgtctacttctatatccgatcgttcctcagtcagcttacgctggaggagcgccagcaggcagccttccatttgcagaagcagatcgagtggctcgaacaagtcctggccagcgccaaggagggtcgtcacctatggtacgaccccgggtgggagaatgatactgaggcccagattgagcacctttgtactgctaactcctaccaccctcatgttcgcctggttcagcgagtcggccaacacctgctccccaccgtacgatcgaacggcaacccattcgaccttctggaccacgatgggctcctgacggagttctataccaacacactcagcttcggacccgcactacactacgcccgggaattggtggcgcagatcgcccatcgctatcagtcaatggatattctggagattggagcagggaccggcggcgctaccaagtacgtgttggccacgccccagctggggttcaacagctacacatacaccgatatctccaccggattcttcgagcaagcgcgggagcaatttgcccccttcgaggaccggatggtgtttgaacccctcgatatccgccgcagtcccgccgagcagggcttcgagccgcatgcctatgatctgatcattgcctccaatgtgctacatgcgacacccgacctagagaaaaccatggctcacgcccgctctctgctcaagcctggaggccagatggttattctggagattacccacaaagaacacacacggctcgggtttatctttggtctgttcgccgactggtgggctggggtggatgatggtcgctgcactgagccgtttgtctcgttcgaccgctgggatgcgatcctaaagcgtgtcgggttttccggtgtggacagtcgcaccacggatcgggacgcaaatctattcccgacctctgtgtttagtacccatgcaattgacgccaccgtggagtacttagacgcgccgcttgccagcagcggcaccgtcaaggactcttaccctcccttggtggtggtaggagggcagaccccccaatctcagcgtctcctgaacgatataaaagcgatcatgcctcctcgtccgctccagacatacaagcgcctcgtggatttgctagacgcggaggagctgccgatgaagtccacgtttgtcatgctcacggagctggacgaggaattattcgccgggctcactgaagagaccttcgaggcaaccaagctgctgctcacgtacgccagcaatacggtctggctgacagaaaatgcctgggtccaacatcctcaccaggcgagcacgatcggcatgctacgctccatccgccgggagcatcctgacttgggagttcatgttctggacgtcgacgcggttgaaaccttcgatgcaaccttcctggttgaacaggtgcttcggcttgaggagcatacggatgagctggccagttcaactacatggactcaagaacccgaggtctcctggtgtaaaggccgcccgtggattcctcgtctgaagcgcgatctggctcgcaataaccgaatgaactcctcgcgccgtcccatatacgagatgatcgattcgtcgcgggctcccgtggcattacagacggctcgggattcatcatcctacttcttggagtccgctgaaacctggtttgtgcctgagagtgttcagcagatggaaacaaagacgatctatgtccactttagctgtccccatgcgcttagggtcggacagctcgggtttttctatcttgtgcagggtcacgtccaggagggcaatcgcgaagtgcccgtcgtggccttagcagagcgtaacgcatccattgtgcacgttcgtcccgattatatatatactgaggcagataacaatctgtctgagggtggtggcagccttatggtaaccgtcctcgccgcggcggtgttggcggagacggtgatcagtaccgccaagtgcctgggggtaactgactcaatcctcgttctgaatccccccagcatatgtgggcagatgttgctccatgctggtgaagagatcggtcttcaagttcatctggccaccacttctggcaacaggagttcggtttctgctggagacgccaagtcctggctaacattgcatgctcgcgacacggactggcacctgcgacgggtactgccccggggtgtccaggctttagtcgacttatcagccgaccagagctgtgaaggtttgactcagaggatgatgaaagttctgatgcctggctgtgcccattaccgtgcggcagacctgttcacagacaccgtttccactgaattgcatagcggatcgcggcatcaagcttcactgcccgccgcatattgggagcatgtggtatccttagcccgccagggacttcctagtgtcagcgaggggtgggaggtgatgccgtgcactcaatttgcagcgcatgccgacaagacgcgcccggatctctcgacagttatttcctggccccgggagtcggacgaggctacgcttcctaccagggttcgctccattgacgctgagaccctctttgcggccgacaaaacatatctcctggtcggactgactggagatcttggacgatcactaggtcgttggatggtccagcatggggcctgccacattgtacttacgagcagaaatccgcaggtgaaccccaagtggctggcgcatgttgaagaactgggtggtcgagtcactgttctttccatggacgtgacaagccaaaactcagtggaagctggcctggctaaactcaaggatctgcatctgccaccagtggggggtattgcctttggccctctggttctgcaggatgtgatgctaaataatatggaactgccaatgatggagatggtgctcaaccccaaggtcgaaggcgtccgcatcctgcacgagaagttctccgatccgaccagtagcaaccctctcgacttcttcgtgatgttctcctcgattgtggccgtcatgggcaacccgggtcaggctaactacagtgcggctaactgctaccttcaagcgctggcgcagcagcgagttgcatccggattagcagcgtccaccatcgacatcggtgccgtgtacggcgttgggttcgtcactcgggcggagctggaggaggactttaatgcaattcggttcatgttcgattcggttgaggaacatgaactgcatacactgtttgctgaggcagtggtggccggtcgacgagccgtgcaccagcaagagcagcagcggaagttcgcgacagtgctcgacatggctgatctggaactgacaaccggaattccgcccctggatccagccctcaaagatcggatcaccttcttcgacgacccccgcataggcaacttaaaaattccggagtaccgaggggccaaagcaggcgaaggggcagccggctccaagggctcggtcaaagaacagctcttgcaggcgacgaacctggaccaggtccgtcagatcgtcatcgatggactctccgcgaagctgcaggtgaccctgcagatccccgatggggaaagcgtgcatcccaccatcccactaatcgatcagggggtggactctctgggcgcggtcaccgtgggaacctggttctccaagcagctgtaccttgatttgccactcctgaaagtgcttgggggtgcttcgatcaccgatctcgctaatgaggctgctgcgcgattgccacctagctccattcccctcgtcgcagccaccgacgggggtgcagagagcactgacaatacttccgagaatgaagtttcgggacgcgaggatactgaccttagtgccgccgccaccatcactgagccctcgtctgccgacgaagacgatacggagccgggcgacgaggacgtcccgcgttcccaccatccactgtctctcgggcaagaatactcctggagaatccagcagggagccgaagaccccaccgtctttaacaacaccattggtatgttcatgaagggctctattgaccttaaacggctgtacaaggcgttgagagcggtcttgcgccgccacgagatcttccgcacggggtttgccaacgtggatgagaacgggatggcccagctggtgtttggtcaaaccaaaaacaaagtccagaccatccaagtgtctgaccgagccggcgccgaagagggctaccgacaactggtgcagacacggtataaccctgccgcaggagacaccttgcggctggtggacttcttctggggccaggacgaccatctgctggttgtggcttaccaccgactcgtcggggatggatctactacagagaacatcttcgtcgaagcgggccagctctacgacggcacgtcgctaagtccacatgtccctcagtttgcggacctggcggcacggcaacgcgcaatgctcgaggatgggagaatggaggaggatctcgcgtactggaagaaaatgcattaccgaccgtcctcaattccagtgctcccactgatgcggcccctggtaggtaacagtagcaggtccgatactccaaatttccagcactgtggaccctggcagcagcacgaagccgtggcgcgacttgatccgatggtggccttccgcatcaaggagcgcagtcgcaagcacaaggcgacgccgatgcagttctatctggcggcgtatcaggtgctgttggcgcgcctcaccgacagcaccgatctcaccgtgggcctcgccgacaccaaccgtgcgactgtcgacgagatggcggccatggggttcttcgccaacctccttcccctgcgcttccgggatttccgcccccatataacgtttggcgagcaccttatcgccacccgtgacctggtgcgtgaggccttgcagcacgcccgcgtgccctacggcgtcctcctcgatcaactggggctggaggtcccggtcccgaccagcaatcaacctgcgcctttgttccaggccgtcttcgattacaagcagggccaggcggaaagtggaacgattgggggtgccaagataaccgaggtgattgccacgcgcgagcgcaccccttacgatgtcgtgctggagatgtcggatgatcccaccaaggatccgctgctcacggccaagttacagagttcccgctacgaggctcaccaccctcaagccttcttggagagctacatgtcccttctctctatgttctcgatgaatcccgccctgaagctggca

SEQ ID NO: 10 - DNA, 1089 bp, Aspergillus terreus
atgggcgaccagccattcattccaccaccgcagcaaacagcgctgacggtaaatgaccatgatgaagtcaccgtctggaatgccgcaccctgccccatgctgccccgcgaccaggtatacgtccgcgtcgaggccgtggcgatcaatcccagtgacacgaagatgcgcggacagtttgccacgccctgggcgtttctcggaacggactatgccggcacggtcgtcgcagtgggttcggacgtgactcatatccaagtgggtgaccgggtctacggggcacagaacgagatgtgcccacgcaccccggatcagggggcattctcgcagtacacggtcacgcgaggccgtgtttgggccaagatccccaagggcttgtcgttcgagcaggctgccgcgctacctgcgggcatcagtaccgctggattggcgatgaagttgcttgggctgcctttgccatcgccttcggcagaccagccacccacccactccaagccggtgtatgtgttggtctatgggggcagtacggccactgccactgtcactatgcaaatgctccgcctgtccggatatattccaattgcaacatgctccccccacaatttcgacctggccaaatcgcgcggcgcagaggaggtctttgactatcgggccccgaatctcgcgcagacgatccgtacctacaccaagaacaatctccgctatgctctcgactgtatcaccaacgtcgagtccaccacattctgcttcgcagccatcggccgcgcgggggggcactacgtctccctgaacccgttccctgaacacgcggccacgcgcaagatggtcacgaccgactggaccctggggccgaccatctttggcgagggatcaacctggcccgccccctatgggcgtcccggcagtgaggaagagcggcagttcggcgaggatctgtggcgcatcgcggggcagctcgtcgaagatggacgcctcgtccatcatccgttgcgcgtggtgcagggcggcttcgatcacattaagcaaggcatggagctcgtccggaagggagagctgtcgggggagaaactcgtggttcggctcgaggggccg

SEQ ID NO: 11 - DNA, 1239 bp, Aspergillus terreus
atgggatccatcattgatgctgctgcggcagcggatccggttgttctgatggaaaccgccttccgcaaggccgtgaaatccaggcagatccccggggcggtcatcatggcccgagattgcagtggcaatctaaattatacgcgctgcttcggggctcggacggtgcgacgggacgagtgcaatcagctgccgccgctacaggtcgacaccccctgccggctcgccagtgcgaccaagctgctgaccacgatcatggccctacaatgcatggagcgcggtctcgtggacttggatgagacggtggataggctgcttccggatttgagcgcgatgcccgtgctggaggggtttgacgacgcgggaaacgcaagattgcgagagcgtcgggggaagatcacgctgcggcacctgctgacgcatacatcgggactgtcgtacgtcttcctccatccgttgctccgggaatacatggcccagggccacctccagtcggcagaaaagtttggcatccagagtcgcctggcgccgccggccgtcaacgaccctggggcggagtggatctacggcgccaacctggactgggcgggtaagctcgtcgagcgggccaccggcctcgacctggagcagtacctgcaggagaatatctgtgcgccgctgggcatcaccgacatgacctttaagctgcagcaacggccggatatgcttgcgcgccgggccgaccaaacgcaccgcaactcggcggatgggcgcctgcgctacgacgactcggtctacttccgggccgatggagaggagtgcttcggcggccagggggtgttctcgggccctgggtcctatatgaaggtgcttcactcgctgttgaagcgagacgggctcctgctgcagccacagaccgtggacttgatgtttcagcctgccctcgagccgcgactcgaagagcagatgaaccagcacatggacgccagcccacatatcaactacggtgggccgatgcccatggtccttcgtcgcagctttgggctgggggggatcatcgccttggaggatctggacggagagaactggcgccgaaaaggttccttgacctttgggggtggcccaaacattgtgtggcaaatcgaccccaaagccggcctgtgcacccttgcgttcttccaactggaaccctggaatgacccggtctgtcgtgatctgacacgcacattcgagcatgccatctatgcgcagtaccagcagggt

SEQ ID NO: 12 - DNA, 7596 bp, Aspergillus terreus
atgacaccattagatgcgcccggtgcgcctgctcccatagctatggttggcatgggctgcagatttggcggaggcgcaacagatccccagaaactgtggaaattgctggaggaaggagggagcgcctggtctaagattcctccttcacgattcaatgtcggcggggtctaccaccccaatggccagcgggtaggatcgatgcacgttcgcggtggacactttctcgacgaagacccggctcttttcgatgcctcatttttcaatatgagtactgaagttgccagttgtatggacccccagtaccgactcatacttgaagtcgtttatgaggcgctcgaagctgcgggaattcctctcgaacaggtctccggctccaagactggggtttttgcaggaaccatgtatcacgactaccaaggctccttccagcgccaaccagaagcccttccacggtatttcataacaggaaatgctggcaccatgctcgcgaatcgcgtctcccacttttatgaccttcgtgggcccagtgtctcgatcgacactgcctgttccacaaccttaacagccttgcatcttgccattcagagcttgcgagctggagaatctgatatggcgattgtcgctggcgcgaacctgctacttaatcctgacgtctttactaccatgtccaaccttggcttcctttcgtccgatgggatttcctactcatttgactcgagagcggatggctatggtcgcggagaaggagtggctgcgatcgtcttgaagactctgcccgatgcggtgcgagacggagacccgatccgcctcatagtgcgcgaaacggcaatcaaccaagacggccggaccccagccatcagcacgccgagcggcgaggcccaggagtgcctgatccaagattgctatcagaaggcccagttggacccaaaacagacttcgtacgttgaggcccatgggacgggaaccagagcaggagatccgctggagcttgcagtcatctcggccgcgtttccgggacagcagatacaggtgggctccgtgaaagccaatatcgggcatacagaggctgtcagtggtctggcgagtttgataaaggtggctctggctgttgaaaagggggttatcccgcctaatgcaaggttcctccagccgagcaagaagttgctcaaggacactcatatccagattccactgtgtagccaatcatggataccaaccgatggtgtccgtcgcgcatcaataaacaacttcggtttcggaggcgcaaatgctcatgcaatcgtggagcaatatggcccgtttgcagaaacatcgatctgcccacctaatggttattctggcaactatgatggcaatttaggaacggatcaagcgcatatatatgtgctgagtgccaaggatgagaacagttgcatgagaatggtttcaaggctgtgcgactatgctacccacgccagaccagccgacgatttgcaattgctcgcgaatatagcatacacgcttggttctcgtcgctcgaacttccgatggaaggcagtatgtacggcacacagcctcacgggtcttgcccagaatttggcgggagaaggcatgcggccaagcaagtcagccgaccaagtaagactgggatgggtgttcacaggccagggagcgcaatggtttgcaatgggtcgtgagttgattgagatgtatcctgtctttaaagaggccctgctggaatgcgatggatatatcaaggaaatggggtcaacctggtccattatagaggaactcagtcgccctgaaacggaaagtcgcgttgatcaggcagaattcagtctgccattgtctacggctcttcaaattgcgcttgttcgtctgctctggtcgtggaacatccaaccagtagccgtcactagtcactccagcggagaggcagctgcagcgtacgctatcggggcactaacagcccgctcggccattggaataagctatatacgcggtgcattgacagcaagagaccgcctggcgtcggtacataaggggggcatgttggctgtcggattgagccgcagtgaagtgggtatatacatcagacaggttccattacagagtgaagaatgcttggtggtggggtgtgtcaacagcccgtcgagtgtgacggtctcgggagatttgtccgccattgccaagttggaggaactgctccatgctgatcgtatatttgcgagacggctgaaagtcacccaagcctttcactccagccacatgaactcgatgacagatgctttccgagccggtcttacagaactcttcggagcagaccccagtgatgcagcaaacgccagtaaagatgtgatctacgcttctcccagaaccggggcccgcctgcacgacatgaatcgtcttcgggatcctatacactgggtcgaatgcatgcttcacccggttgagttcgaatcagcattccgtcgaatgtgcctggacgaaaacgaccacatgccaaaggtcgatagggtcattgagattggacctcacggagcgcttggaggcccgatcaagcagatcatgcagcttccagagcttgccacgtgtgacatcccttatctgtcctgtctttctcgtgggaagagctctctgagcacccttcgccttctcgcatcagaacttatccgggccggatttcctgttgacttgaatgcgatcaactttccccgcggatgtgaagcagctcgggtccaagtgttgtctgatctaccgccctacccttggaaccacgagaccagatactggaaagagccgcgcatcagccaatctgcccggcagcggaagggcccagtccacgatctgatcggattgcaggagccgttgaacctgccgttggcgcggtcatggcacaatgtgcttcgtgtgtcagatttgccatggctacgcgaccacgtcgtcggctcgcatattgttttccctggggctgggttcgtgtgtatggcagtgatgggaatcagcacgctctgctcgtccgaccatgaatctgacgacatcagttacatcctacgcgacgtgaactttgcgcaggccctgattctacctgcggacggggaagaaggaatagatctgcgcctcacgatttgtgctcccgatcagagtctgggttcacaggactggcaaagattcttagttcattcgatcactgctgacaagaatgactggacggaacactgtacgggacttgttcgagcagagatggaccagcctccctccagtttgtcgaaccaacaacggatagacccacggccatggagccgtaaaacggcgccgcaggagctgtgggactcactacatcgggtgggaattcgtcatgggcccttttttcgaaacattacgtgcatcgaaagcgacgggcgagggtcatggtgtacatttgccatcgcggacacggcctccgcaatgccacacgcctacgaatcccagcacattgttcacccaaccacactagactctgcagttcaggcagcctataccactcttccattcgctgggagccggatcaaatctgcgatggtccccgctcgcgtcggctgcatgaagatttcctcccgacttgcagatttggaggccagggacatgctgcgcgcacaagcgaagatgcacagccaaagtccttccgcattggtaaccgatgtagcagtttttgatgaggcagatccggttggagggcctgttatggagctcgaagggctggtctttcagtctctgggggcaagtctgggcacttctgaccgggactccaccgaccccgggaatacttgcagctcctggcattgggctccagacatcagcttagttaaccccggctggcttgaaaaaaccctgggcacaggtattcaggagcacgagatcagcctcatattggagcttcgacggtgttcggtgcacttcattcaagaggccatggaaagtttgagcgtaggcgatgtcgagaggctgagtggtcatctggccaaattctatgcgtggatgcagaaacaactggcgtgtgcccaaaatggcgagctggggccagagagctccagctggactcgggatagcgagcaggcaagatgcagcctccgctctagagtggttgctggtagcaccaacggcgaaatgatctgtcgcctgggctccgtgctccccgctatcctacgtcgggaagttgatccgttggaggtgatgatggatggccacctgttgtcccgctactatgtcgatgccctcaagtggagtcggtccaacgcgcaagccagcgagctcgtgcgcctctgctgccacaaaaacccgcgcgctcgcatactggaaatcggcggaggcaccgggggttgcacccagctggtcgtggactccttgggcccaaatccgccggtaggccgctatgactttactgacgtctcggccgggttttttgaagcagcccgcaagcggttcgcgggatggcagaatgtgatggattttcggaagttggacatcgaggacgatccagaagcgcaggggtttgtgtgcggatcctacgacgtggtgttggcttgtcaggtcctgcatgccacttctaacatgcagcgcacattgactaatgtgcgcaagctgttgaagccaggaggcaaactcattcttgtcgaaaccaccagagacgagcttgacttgtttttcactttcgggcttctgcccggctggtggctcagcgaagaaccagaaagacagtcgactccgtcactaagccctacgatgtggcgcagcatgctgcacactactggattcaatggtgtggaagttgaggctcgtgactgcgatagccacgagttctatatgattagcaccatgatgtccacggccgtacaggcgactccgatgtcatgctcggtcaaattgcctgaagtgctcttggtctatgttgactcatctacgcccatgtcttggatatcagatttgcagggagagattcgcggcaggaattgttccgtcacttcgctacaggcacttcgtcaagttcctcctaccgagggccaaatatgcgtattccttggagaggtggaacactccatgcttggttcagtcaccaacgacgacttcacacttttgacctcaatgctacagctggctgggggaactttatgggtcacccaaggagcgacaatgaagtctgatgatcccctgaaggctctacacctcggattactacgtaccatgcgtaatgaaagccatggcaagcgatttgtctcacttgacctcgacccttcgcgtaatccatggacaggcgattcgcgcgatgccattgtcagtgttctggatttaattagcatgtcagatgaaaaggagtttgactatgcagagcgggatggagttatccatgttcctcgggcatttagtgactccatcaatggaggcgaggaagacgggtatgccttggagccattccaggacagccagcatctcctgcgactagatatacagactcctgggctcctcgattccctgcacttcacaaagcgcaatgtggacacatatgaaccagataaattaccggacgactgggtagagattgaaccgagggcgtttggtcttaacttccgtgacatcatggtcgcgatgggtcaattggaatcaaacgtcatgggcttcgaatgcgccggcgtggttacaagtctcagcgagacagcaagaacaattgcacccgggcttgcggtcggagatcgggtttgcgccctcatgaacggacactgggcgtcgagggtgaccacaagccggaccaacgtggtgcgcattccagagactcttagtttcccgcatgctgcctccatccctctggccttcacaacagcttacatttcactttacaccgttgcccgcattctgccaggtgaaacggtgttgatccatgccggggcaggaggcgtaggccaggcggccattattcttgctcaattaaccggtgctgaagtctttacaactgctggcagtgagaccaagcgtaaccttttgatcgataaattccacctcgaccctgatcatgtcttctcgagcagggactccagcttcgtcgacggtatcaagacccgcacccgtggcaagggggtggacgtggttttgaactcgctagctgggcctctccttcagaagagctttgactgtctggctaggtttggtcggtttgtagaaatcggcaagaaggatcttgagcagaatagccgactcgacatgtcgacgttcgtccgcaatgtctccttctcctccgttgatattctctactggcagcaagcgaagcccgctgaaatcttccaggcgatgtccgaggtcatcttgctgtgggagcgaacggcaatcggcctgattcatccaatatcagagtatcctatgtcggccctggagaaggcctttcgcactatgcagagcggccagcacgttgggaagattgttgtgacagtagcccccgatgacgcggtcctcgttcgtcaggaacgaatgccactatttctgaagcctaacgtgtcgtatcttgttgctgggggcctgggtggtatcggacggcggatctgcgagtggctggtcgatcgcggggcgcgatatctcatcattctgtctcgaactgctcgcgtggacccggtcgtgacgagtctccaagagcggggctgcaccgtttctgtacaggcgtgtgatgtggccgatgaaagccagcttgaagcggctctccaacagtgtcgggcggaggaaatgcctccgattcggggcgtcatccaaggggcaatggttctcaaggacgccctcgtctcgcaaatgacggcggacgggttccatgccgccctgcggcccaaggttcagggaagttggaatctgcaccgaattgcatcggacgtggatttcttcgtgatgctctcatccttggtgggtgtcatgggaggcgcaggacaagccaactacgcggctgccggagcgtttcaggacgcgctcgcagagcaccgcatggctcacaaccagccagcggtcaccatcgacctcggaatggtccagtcaattgggtatgtagcagagacagattctgctgtggcggaacgactccaacggatcggctatcaacccttgcacgaagaggaggttctggacgtcctcgagcaagctatatctcctgtgtgttcccctgccgcacccacacggcctgctgtcatcgtcaccggcatcaacacccgcccaggccctcactgggcacacgccgactggatgcaagaggctcgctttgcggggatcaagtatcgtgatccgttgagggacaatcatggagctttgtcgctgaccccggcggaagatgacaatcttcacgccaggctgaaccgtgcaatcagccaacaggagtcaatcgccgtgatcatggaggcgatgagctgcaagctcatctcaatgttcggcctgacggatagcgaaatgtccgccactcagacattggcggggatcggcgtggactccctggtcgccattgagctccggaactggatcacagctaagttcaatgttgatatctcagttttcgagttgatggagggccgaacgatcgccaaagtcgcggaagtggtgctgcagagatacaaagct

SEQ ID NO: 13 - DNA, 768 bp, Aspergillus terreus
atgcgctaccaagcatctccagcactggcgaaggcgccccgagcgcttctttgcatccatggggctggctgctctcccgccatcttccgcgtgcaattgtctaagctccgggctgcgctgcgcgaagactttgaattcgtctacgtgacagccccgttcccttcctctgcagggcctgggattctccccgtcttcgccgacctggggccatattactcatggtttgaaagcagcagcggcaacaataataatgggccctccgtgggcgaacgcctcgccgccgtccacgaccccatccgccgcaccattgtcgactggcagactcaacacccccacatccctatcgtgggtgctatcggtttctccgaaggtgccctggtgacgaccttgctcctctggcagcagcagatgggtcgcctgccctggttgccccggatgagtgtcgcgatgttgatctgcccctggtatcaagacgaggcaagccagtatatgaggaacgaagtgatggagaaccatgacgacgaccacgacagcaaagataccgagtggcaggaggaactggtcattcggataccgacattacatctgcagggtcgcgatgattttgcgctcgcaggatcgaagatgctggtggcgcgccatttctccccccgagaggcgcaggtattggagtttgctgggcagcatcagtttcccaatcgaccgcgcgacgtgttggaggttattaatcgttttcgtaagctgtgtgtgacggtccagacattggag

SEQ ID NO: 14 - DNA, 2085 bp, Aspergillus terreus
atggctcaactcgacactctcgacctggtggtcctggtggtgcttttggtgggtagcgccgcctacttcaccaagggcacctactgggccgttcccaaggacccgtatgccgcctccggtcccgccatgaatggtggcgccaaggcgggcaaatccagggacatcattgagaaaatggaagagactggcaagaactgtgtgattttctacggctcgcagaccggtaccgccgaggattatgcgtcgcgcctggccaaggaaggctcccagcgtttcggcctcaagaccatggtcgcagatctggaagactacgattatgagaacctggacaagttccccgaggacaaggttgccttcttcgtcatggccacctatggtgagggtgaacccaccgacaacgccgtcgagttctaccagttcatctcgggtgaggacgtcgcgttcgagagcggcgcctctgccgacgacaagcccctgtcctccctcaagtatgtcactttcggtctcggtaacaacacctatgagcactatcaggctatggttcgcaatctggatgccgctctcaccaagctgggtgcgcagcgcattggagatgctggtgaaggtgatgacggcgctggcaccatggaagaagatttcctggcctggaaagagcccatgtggactgccctgtccgaggccatgaaccttcaggagcgcgaagccgtctatgagccggtgttctcggtcacggaagatgaatccctgtcccccgaagacgaagccgtctacctcggtgagccgaccaagggtcaccgtgacggcacccccagtggcccgtattccgctcacaaccccttcatcgcccccatcgtcgagtctcgtgaactgttcaacgtcaaggaccgtaattgtctgcacatggagatcagcatcgctggtagcaacctttcttaccagactggtgatcacatcgcgatttggcccacgaacgctggtgccgaggtggaccggttcctccaggtgtttggtcttgagaacaagcgtcattccgtcatcaacatcaagggtatcgatgtgaccgccaaggttcccattccgactcccaccacgtatgatgctgctgttcgctactatatggaaattgctgcgcccgtctcccgtcagtttgtggctaccctggctgcgtttgctcccgatgaggagactaaggcggaaatcgtgcgtttgggtagcgacaaggactacttccacgagaaaatcagcaaccagtgcttcaccatcgctcaggctcttcagagtgtcacctccaagcccttctcggctgtcccgttctctctgcttatcgagggtctcaataagctccagccccgttactactccatctcttcctcctccatggtccagaaggataagatcagcattactgccgtcgtggaatccactcgcttgcctggtgccgcccaccttgtcaagggtgtcacgaccaactatctccttgccctgaagcaaaagcagaatggggatccgtctcccgaccctcacggcttaacttatactatcactgggccccgtaacaagtacgacggaatccacgttcccgttcacgtccgccactccaatttcaagctcccctctgatccctctcggcccattatcatggttggccctggtaccggtgtggctcccttccgtggattcatccaggagcgtgccgccttggccgccaagggtgagaacgtcggtcccaccgtgttgttctttggatgccgcaggcgcgatgaggactttatgtacgcagatgaattcaagacctaccaggaacagcttggggacaagcttcagatcattactgcgttttctcgtgaaacttcccagaaggtgtatgttcagcacagactgcgtgaacactccgatctggtgagcagcctcctgaagcagaaggctaacttttacgtctgcggtgacgccgccaacatggcgcgtgaagtcaaccttgtgcttggccagatcatcgcgcaacagcggggtctcccggctgaacgggccgaggaaatggtgaagcacatgcgcagcagcggcagctaccaggaggacgtgtggtca

SEQ ID NO: 15 - protein, 6 aa, Artificial Sequence
  Description of Artificial Sequence Synthetic 6xHis tag
HHHHHH

SEQ ID NO: 16 - protein, 232 aa, Aspergillus terreus
AKAPRALLCIHGAGCSPAIFRVQLSKLRAALREDFEFVYVTAPFPSSAGPGILPVFADLGPYYSWFESSSGNNNNGPSVGERLAAVHDPIRRTIVDWQTQHPHIPIVGAIGFSEGALVTTLLLWQQQMGRLPWLPRMSVAMLICPWYQDEASQYMRNEVMENHDDDHDSKDTEWQEELVIRIPTLHLQGRDDFALAGSKMLVARHFSPREAQVLEFAGQHQFPNRPRDVLEV

SEQ ID NO: 17 - protein, 209 aa, Unknown
  Description of Unknown Serine hydrolase (FSH1) family polypeptide
MSKKLKILCLHGFGQSGEIFRAKTGALRKLLKKGVELVYLDAPFPLAEKADLPFEESDAEEGEDDEPYRAWFFGDDDTNEYRGLDESLDYVRDYIKENGPFDGILGFSQGAALAAILASLLEEGLPLESHPPLKFAILISGFRPREPKYAEYYSPPIQTPSLHVIGELDTVVPEERSEKLAEACKNSATVLEHPGGHFVPNKKPFVEAI

SEQ ID NO: 18 - DNA, 48 bp, Artificial Sequence
  Description of Artificial Sequence Synthetic primer
aacatatgcaccatcatcaccaccacatgcgctaccaagcatctccag

SEQ ID NO: 19 - DNA, 32 bp, Artificial Sequence
  Description of Artificial Sequence Synthetic primer
aagcggccgctcactccaatgtctggaccgtc

SEQ ID NO: 20 - DNA, 27 bp, Artificial Sequence
  Description of Artificial Sequence Synthetic primer
aacatatgcgctaccaagcatctccag

SEQ ID NO: 21 - DNA, 50 bp, Artificial Sequence
  Description of Artificial Sequence Synthetic primer
aagtttaaactcagtggtggtgatgatggtgctccaatgtctggaccgtc

SEQ ID NO: 22 - DNA, 20 bp, Artificial Sequence
  Description of Artificial Sequence Synthetic primer
cagatggactctccgcgaag

SEQ ID NO: 23 - DNA, 22 bp, Artificial Sequence
  Description of Artificial Sequence Synthetic primer
tcaccgtgggaacctggttctc

SEQ ID NO: 24 - DNA, 45 bp, Artificial Sequence
  Description of Artificial Sequence Synthetic primer
cgtccgtccgtctctccgcatggtgtgacggcccagacattggag

SEQ ID NO: 25 - DNA, 21 bp, Artificial Sequence
  Description of Artificial Sequence Synthetic primer
catgcggagagacggacggac

SEQ ID NO: 26 - DNA, 21 bp, Artificial Sequence
  Description of Artificial Sequence Synthetic primer
tcagtcctgctcctcggccac

SEQ ID NO: 27 - DNA, 43 bp, Artificial Sequence
  Description of Artificial Sequence Synthetic primer
cgtggccgaggagcaggactgaggagatgcttggtaacgcatg

SEQ ID NO: 28 - DNA, 21 bp, Artificial Sequence
  Description of Artificial Sequence Synthetic primer
ctgtagaagacaatgagcttg

SEQ ID NO: 29 - DNA, 21 bp, Artificial Sequence
  Description of Artificial Sequence Synthetic primer
taatccgcatagcagccatgc

SEQ ID NO: 30 - DNA, 20 bp, Artificial Sequence
  Description of Artificial Sequence Synthetic primer
ggcctggacgagctgtacgc

SEQ ID NO: 31 - DNA, 21 bp, Artificial Sequence
  Description of Artificial Sequence Synthetic primer
ctgatgaacagggtcacgtcg

SEQ ID NO: 32 - DNA, 34 bp, Artificial Sequence
  Description of Artificial Sequence Synthetic primer
tcaaggaagcttgcaaaacgtaggggcaaacaaa

SEQ ID NO: 33 - DNA, 36 bp, Artificial Sequence
  Description of Artificial Sequence Synthetic primer
cgaactaagcttgggagcaaaaagtagaatattatc